(19) United States
(12) Patent Application Publication
(10) Pub. No.: US 2010/0063013 A1
(43) Pub. Date: Mar. 11, 2010

(54) CATHEPSIN CYSTEINE PROTEASE INHIBITORS

(76) Inventors: Renata Oballa, Kirkland (CA); Christopher Bayly, Beaconsfield (CA); Jean-Francois Truchon, Saint-Laurent (CA); Chun Sing Li, Dollard-Des-Ormeaux (CA); Serge Leger, Nortre-Dame- (CA)

Correspondence Address: MERCK AND CO., INC, P O BOX 2000, RAHWAY, NJ, 07065-0907, US

(21) Appl. No.: 12/532,652

(22) PCT Filed: Mar. 25, 2008
(86) PCT No.: PCT/CA08/00559

§ 371 Date: Sep. 23, 2009

Related U.S. Application Data
(60) Provisional application No. 60/919,999, filed on Mar. 26, 2007.

(51) Int. Cl. A61K 31/395; C07D 285/00; A61K 31/496; A61P 3/00; A61P 29/00; A61K 31/59; A61K 31/663

(52) U.S. Cl. 514/167; 540/454; 514/431; 514/252.13; 514/141

(57) ABSTRACT

The present invention relates to novel compounds of the formula (I), wherein R′-R7, X, Y, D and n are as defined in the specification. These compounds are cysteine protease inhibitors which include but are not limited to inhibitors of cathepsms K, L, S and B and are useful for treating diseases in which inhibition of bone resorption is indicated, such as osteoporosis.

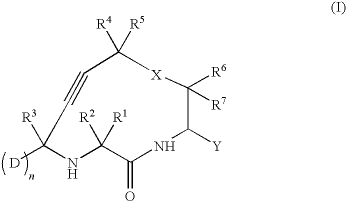

### BACKGROUND OF THE INVENTION

A variety of disorders in humans and other mammals involve or are associated with abnormal bone resorption. Such disorders include, but are not limited to, osteoporosis, glucocorticoid induced osteoporosis, Paget's disease, abnormally increased bone turnover, periodontal disease, tooth loss, bone fractures, rheumatoid arthritis, osteoarthritis, periprosthetic osteolysis, osteogenesis imperfecta, hypercalcemia of malignancy or multiple myeloma. One of the most common of these disorders is osteoporosis, which in its most frequent manifestation occurs in postmenopausal women. Osteoporosis is a systemic skeletal disease characterized by a low bone mass and microarchitectural deterioration of bone tissue, with a consequent increase in bone fragility and susceptibility to fracture. Osteoporotic fractures are a major cause of morbidity and mortality in the elderly population. As many as 50% of women and a third of men will experience an osteoporotic fracture. A large segment of the older population already has low bone density and a high risk of fractures. There is a significant need to both prevent and treat osteoporosis and other conditions associated with bone resorption. Because osteoporosis, as well as other disorders associated with bone loss, are generally chronic conditions, it is believed that appropriate therapy will typically require chronic treatment.

Cathepsins belong to the papain superfamily of cysteine proteases. These proteases function in the normal physiological as well as pathological degradation of connective tissue. Cathepsins play a major role in intracellular protein degradation and turnover and remodeling. To date, a number of cathepsin have been identified and sequenced from a number of sources. These cathepsins are naturally found in a wide variety of tissues. For example, cathepsin B, C, F, H, L, K, O, S, V, W, and Z have been cloned. Cathepsin L is implicated in normal lysosomal proteolysis as well as several diseases states, including, but not limited to, metastasis of melanomas. Cathepsin S is implicated in Alzheimer's disease, atherosclerosis, chronic obstructive pulmonary disease and certain autoimmune disorders, including, but not limited to juvenile onset diabetes, multiple sclerosis, pemphigus vulgaris, Graves' disease, myasthenia gravis, systemic lupus erythemotasus, rheumatoid arthritis and Hashimoto's thyroiditis; allergic disorders, including, but not limited to asthma; and allogenic immune responses, including, but not limited to, rejection of organ transplants or tissue grafts. Increased Cathepsin B levels and redistribution of the enzyme are found in tumors, suggesting a role in tumor invasion and metastasis. In addition, aberrant Cathepsin B activity is implicated in such disease states as rheumatoid arthritis, osteoarthritis, pneumocystisis carinii, acute pancreatitis, inflammatory airway disease and bone and joint disorders.

Mammalian cathepsins are related to the papain-like cysteine proteases expressed by disease-causing parasites including those from the families protozoa, platyhelminthes, nematodes and arthropodes. These cysteine proteases play an essential role in the life cycle of these organisms.

Human type I collagen, the major collagen in bone is a good substrate for cathepsin K. See Kafienah, W., et al., 1998, Biochem J 331:727-732, which is hereby incorporated by reference in its entirety. In vitro experiments using antisense oligonucleotides to cathepsin K, have shown diminished bone resorption in vitro, which is probably due to a reduction in translation of cathepsin K mRNA. See Inui, T., et al., 1997, J Biol Chem 272:8109-8112, which is hereby incorporated by reference in its entirety. The crystal structure of cathepsin K has been resolved. See McGrath, M. E., et al., 1997, Nat Struct Biol 4:105-109; Zhao, B., et al., 1997, Nat Struct Biol 4: 109-11, which are hereby incorporated by reference in their entirety. Also, selective peptide based inhibitors of cathepsin K have been developed See Bromme, D., et al., 1996, Biochem J 315:85-89; Thompson, S. K., et al., 1997, Proc Natl Acad Sci USA 94:14249-14254, which are hereby incorporated by reference in their entirety. Accordingly, inhibitors of Cathepsin K can reduce bone resorption. Such inhibitors would be useful in treating disorders involving bone resorption, such as osteoporosis.

What is needed in the art are therapeutic agents to treat diseases associated with cathepsin activity. Diseases associated with Cathepsin K include: osteoporosis, glucocorticoid induced osteoporosis, Paget's disease, abnormally disease, tooth loss, bone fractures, rheumatoid arthritis, osteoarthritis, periprosthetic osteolysis, osteogenesis imperfecta, atherosclerosis, obesity, glaucoma, chronic obstructive pulmonary disease and cancer including metastatic bone disease, hypercalcemia of malignancy, and multiple myeloma. Diseases associated with Cathespin S include: Alzheimer's disease, atherosclerosis, neuropathic and inflammatory pain, obesity, diabetes, chronic obstructive pulmonary disease, cancer and certain autoimmune disorders, including, but not limited to juvenile onset diabetes, multiple sclerosis, pemphigus vulgaris, Graves' disease, myasthenia gravis, systemic lupus erythemotasus, rheumatoid arthritis and Hashimoto's thyroiditis; allergic disorders, including, but not limited to asthma; and allogenic immune responses, including, but not limited to, rejection of organ transplants or tissue grafts. Diseases associated with Cathepsin B include: tumor invasion, metastasis, rheumatoid arthritis, osteoarthritis, liver diseases, stroke, Alzheimer's disease, viral infections, inflammatory bowel disease, pneumocystis carinii, acute pancreatitis, inflammatory airway disease, bone and joint disorders, and chronic obstructive pulmonary disease (COPD). Diseases associated with Cathepsin L include: tumor invasion, metastasis, osteoarthritis, stroke, viral infections, inflammatory bowel disease, type I diabetes and obesity.

### SUMMARY OF THE INVENTION

The present invention relates to compounds that are capable of treating and/or preventing cathepsin dependent conditions or disease states in a mammal in need thereof. One embodiment of the present invention is illustrated by a compound of Formula I, and the pharmaceutically acceptable salts, stereoisomers and N-oxide derivatives thereof:

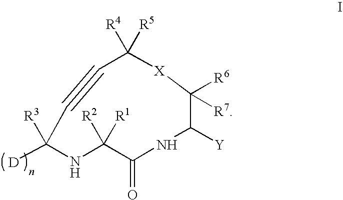

### DETAILED DESCRIPTION OF THE INVENTION

The present invention relates to compounds of the following chemical formula:

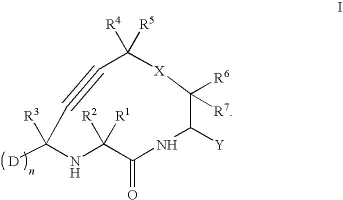

wherein Y is hydrogen, CN, —C(O)R⁸, —C(O)NR⁸R⁹, —CH₂OH, —C(O)NR⁸OR⁹, or —C(O)OR⁸;

### X is S(O)ₘ, —CH₂—, —OC(O)— or —C(O)O—;

R¹ is hydrogen, C₁₋₆ alkyl or C₂₋₆ alkenyl wherein said alkyl and alkenyl groups are optionally substituted with SO₂R¹⁰, C₃₋₆ cycloalkyl or halo;
R² is hydrogen, C₁₋₆ alkyl or C₂₋₆ alkenyl wherein said alkyl and alkenyl groups are optionally substituted with SO₂R¹⁰, C₃₋₆ cycloalkyl or halo;
or R¹ and R² can be taken together with the carbon atom to which they are attached to form a C₃₋₈ cycloalkyl ring, C₅₋₈ cycloalkenyl ring, or five to seven membered heterocyclyl wherein said cycloalkyl, cycloalkenyl and heterocyclyl groups are optionally substitutedon either the carbon or heteroatom with C₁₋₆ alkyl, halo, hydroxyalkyl, hydroxy, alkoxy or keto;
R³ is C₁₋₆ alkyl substituted with 1-6 halo;
R⁴ is hydrogen, C₁₋₆ alkyl or C₂₋₆ alkenyl wherein said alkyl and alkenyl groups are optionally substituted with C₃₋₆ cycloalkyl or halo;
R⁵ is hydrogen, C₁₋₆ alkyl or C₂₋₆ alkenyl wherein said alkyl and alkenyl groups are optionally substituted with C₃₋₆ cycloalkyl or halo;
or R⁴ and R⁵ can be taken together with the carbon atom to which they are attached to form a C₃₋₈ cycloalkyl ring, C₅₋₈ cycloalkenyl ring, or five to seven membered heterocyclyl wherein said cycloalkyl, cycloalkenyl and heterocyclyl groups are optionally substituted on either the carbon or heteroatom with C₁₋₆ alkyl, halo, hydroxyalkyl, hydroxy, alkoxy or keto;
R⁶ is hydrogen, C₁₋₆ alkyl or C₂₋₆ alkenyl wherein said alkyl and alkenyl groups are optionally substituted with C₃₋₆ cycloalkyl or halo;
R⁷ is hydrogen, C₁₋₆ alkyl or C₂₋₆ alkenyl wherein said alkyl and alkenyl groups are optionally substituted with C₃₋₆ cycloalkyl or halo;
or R⁶ and R⁷ can be taken together with the carbon atom to which they are attached to form a C₃₋₈ cycloalkyl ring, C₅₋₈ cycloalkenyl ring, or five to seven membered heterocyclyl wherein said cycloalkyl, cycloalkenyl and heterocyclyl groups are optionally substituted on either the carbon or heteroatom with C₁₋₆ alkyl, halo, hydroxyalkyl, hydroxy, alkoxy or keto;
R⁸ is hydrogen, C₁₋₆ alkyl or C₂₋₆ alkenyl wherein said alkyl and alkenyl groups are optionally substituted with C₃₋₆ cycloalkyl or halo;
R⁹ is hydrogen, C₁₋₆ alkyl or C₂₋₆ alkenyl wherein said alkyl and alkenyl groups are optionally substituted with C₃₋₆ cycloalkyl or halo;
or R⁸ and R⁹ can be taken together with the atoms to which they are attached or are between them to form a C₃₋₈ cycloalkyl ring, C₅₋₈ cycloalkenyl ring, or five to seven membered heterocyclyl wherein said cycloalkyl, cycloalkenyl and heterocyclyl groups are optionally substituted on either the carbon or heteroatom with C₁₋₆ alkyl, halo, hydroxyalkyl, hydroxy, alkoxy or keto;
R¹⁰ is C₁₋₆ alkyl, C₁₋₆ haloalkyl, C₃₋₆ cycloalkyl, C₃₋₆ cycloalkyl(C₁₋₆ alkyl), aryl, aryl(C₁₋₆ alkyl), heteroaryl or heteroaryl(C₁₋₆ alkyl), wherein said cycloalkyl group is optionally substituted with C₁₋₆ haloalkyl, and wherein said aryl and heteroaryl groups are optionally substituted with 1 to 3 substituents independently selected from C₁₋₆ alkyl, halo, C₁₋₆ haloalkyl, —SR^{a}, —S(O)R^{a}, —S(O)₂R^{a}, —OR^{a}, NR^{c}R^{d}, cyano or aryl;
R^{a} is hydrogen, C₁₋₆ alkyl, aryl, heteroaryl, aryl(C₁₋₆ alkyl), or heteroaryl(C₁₋₆ alkyl);
R^{b} is hydrogen or C₁₋₆ alkyl;
R^{e} is hydrogen or C₁₋₆ alkyl;
R^{d} is hydrogen or C₁₋₆ alkyl;
or R^{c} and R^{d} can be taken together with the nitrogen atom to which they are attached to form a four to six membered heterocyclyl which may contain a second heteroatom selected from O, S, NH or NC₁₋₆ alkyl;
Each D is independently hydrogen, C₂₋₆ alkynyl, aryl, heteroaryl, C₃₋₈ cycloalkyl or heterocyclyl wherein said alkynyl, aryl, heteroaryl, cycloalkyl and heterocyclyl groups, which may be monocyclic or bicyclic, are optionally substituted on either the carbon or the heteroatom with one to five R¹¹;
R¹¹ is hydrogen, C₁₋₆ alkyl, C₂₋₆ alkenyl, C₂₋₆ alkynyl, C₁₋₆ alkyloxy, halo, nitro, cyano, aryl, heteroaryl, C₃₋₈ cycloalkyl, heterocyclyl, —C(O)OR¹³, —OR¹⁵, —OR¹³, —C(O)R¹³, R¹³C(O)R¹⁵, —C(O)N(R^{a})(R^{b}), —C(O)N(R¹³)(R¹⁴), —C(R¹³)(R¹⁴)OH, —R¹⁵, —C(R¹³)(R¹⁴)N(R¹⁵)₂, —NR¹⁰C(O)NR¹³S(O)₂R¹⁵, —SO₂R¹², —SO(R¹²), —SO₂R¹⁵, —SOₘN(R^{c})(R^{d}), —SOₘCH(R¹³)(R¹⁴), —SO₂N(R¹³)C(O)(R¹²), —N(R¹³)(R¹⁴), —N(R¹³)C(O)N(R¹³)(R¹⁵), —N(R¹³)C(O)R¹⁵, —N(R¹³)C(O)R¹³, —N(R¹³)C(O)OR¹³, —N(R¹³)SO₂(R¹³), —C(O)C(R^{a})(R^{b})N(R^{c})(R^{d}), —C(R^{a})(R^{b})N(R^{c})C(O)R¹⁵, —C(O)C(R^{a})(R^{b})S(R^{a}), C(R^{a})(R^{b})C(O)N(R^{c})(R^{d}); wherein said groups are optionally substituted on either the carbon or the heteroatom with one to five substituents independently selected from C₁₋₆ alkyl, halo, keto, cyano, C₁₋₆ haloalkyl, hydroxyalkyl, —OR¹⁵, —NO₂, —NH₂, —NHS(O)₂R¹³, —R¹⁵SO₂R¹², —SO₂R¹², —SO(R¹²), —SR¹², —SR¹⁵, —SOₘN(R^{c})(R^{d}), —SOₘN(R¹³)C(O)(R¹²), —C(R¹³)(R¹⁴)NR¹³)(R¹⁴), —C(R¹³)(R¹⁴)OH, —COOH, —C(R^{a})(R^{b})C(O)N(R^{c})(R^{d}), —C(O)(R^{a})(R^{b}), —N(R¹³)C(R¹³)(R¹⁴)(R¹⁵), —NR¹³)CO(R¹⁵), —NH(CH₂)₂OH, —NHC(O)OR¹³, heterocycyl, aryl, or heteroaryl;
R¹² is hydrogen or C₁₋₆ alkyl which is optionally substituted with one, two, or three substituents independently selected from halo, alkoxy, cyano, —NR¹³ or —SR¹³;
R¹³ is hydrogen or C₁₋₆ alkyl;
R¹⁴ is hydrogen or C₁₋₆ alkyl;
R¹⁵ is hydrogen, aryl, aryl(C₁₋₄) alkyl, heteroaryl, heteroaryl(C₁₋₄)alkyl, C₃₋₈cycloalkyl, C₃₋₈ cycloalkyl(C₁₋₄)alkyl or heterocyclyl(C₁₋₄)alkyl wherein said groups can be optionally substituted with one, two, or three substituents independently selected from halo, alkoxy or —SO₂R¹²;
m is 0, 1, or 2;
n is 1, 2 or 3;
or a pharmaceutically acceptable salt, stereoisomer or N-oxide derivative thereof.

An embodiment of the invention is a compound of the formula:

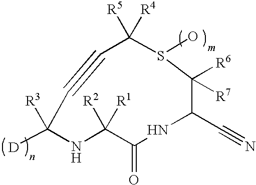

wherein all variables are as defined above.

Another embodiment of the invention is a compound of the formula:

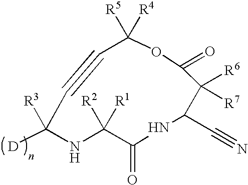

wherein all variables are as defined above.

Another embodiment of the invention is a compound of the formula:

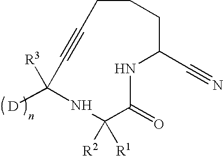

wherein all variables are as defined above.

In an embodiment of the invention, X is S.

In an embodiment of the invention, R³ is C₁₋₃ alkyl substituted with one to three halo. In a class of the invention, R³ is trifluoromethyl.

In an embodiment of the invention, R⁴ is hydrogen or C₁₋₃ alkyl.

In an embodiment of the invention, R⁵ is hydrogen.

In an embodiment of the invention, R⁶ is hydrogen.

In an embodiment of the invention, R⁷ is hydrogen.

Reference to the preferred embodiments set forth above is meant to include all combinations of particular and preferred groups unless stated otherwise.

Specific embodiments of the present invention include, but are not limited to:
(3R,6S,8R)-8-(4-bromophenyl)-6-(2-fluoro-2-methylpropyl)-5-oxo-8-(trifluoromethyl)-1-thia-4,7-diazacycloundec-9-yne-3-carbonitrile 1,1-dioxide;
(3R,6S,8R)-8-(1-benzothien-2-yl)-6-isobutyl-5-oxo-8-(trifluoromethyl)-1-thia-4,7-diazacycloundec-9-yne-3-carbonitrile;
(3R,6S,8R)-3-acetyl-8-(4-bromophenyl)-6-(2-fluoro-2-methylpropyl)-8-(trifluoromethyl)-1-thia-4,7-diazacycloundec-9-yn-5-one 1,1-dioxide;
(3R,6S,8R)-3-acetyl-8-(4-bromophenyl)-6-(2-fluoro-2-methylpropyl)-8-(trifluoromethyl)-1-thia-4,7-diazacycloundec-9-yn-5-one;
(3R,6S,8R)-8-(1-benzothien-2-yl)-6-isobutyl-5-oxo-8-(trifluoromethyl)-1-thia-4,7-diazacycloundec-9-yne-3-carboxamide;
(3R,6S,8R)-8-(4-bromophenyl)-11-ethyl-6-(2-fluoro-2-methylpropyl)-5-oxo-8-(trifluoromethyl)-1-thia-4,7-diazacycloundec-9-yne-3-carbonitrile;
(2S,5S,11R)-11-(4-bromophenyl)-2-(2-fluoro-2-methylpropyl)-3-oxo-11-(trifluoromethyl)-1,4-diazacycloundec-9-yne-5-carbonitrile;
(3R,6S,8R)-8-(4-bromophenyl)-11-ethyl-6-(2-fluoro-2-methylpropyl)-5-oxo-8-(trifluoromethyl)-1-thia-4,7-diazacycloundec-9-yne-3-carboxamide;
(2S,5S,11R)-11-(4-bromophenyl)-2-(2-fluoro-2-methylpropyl)-3-oxo-11-(trifluoromethyl)-1,4-diazacycloundec-9-yne-5-carboxamide;
(3S,5R,11S)-5-(4-bromophenyl)-3-(2-fluoro-2-methylpropyl)-11-(hydroxymethyl)-5-(trifluoromethyl)-1,4-diazacycloundec-6-yn-2-one;
(2S,5S,11R)-11-(4-bromophenyl)-2-(2-fluoro-2-methylpropyl)-3-oxo-11-(trifluoromethyl)-1,4-diazacycloundec-9-yne-5-carboxylic acid;
(4S,7S,9R)-9-(4-bromophenyl)-74 sobutyl-2,6-dioxo-9-(trifluoromethyl)-1-oxa-5,8-diazacyclododec-10-yne-4-carbonitrile;
(3R,6S,8R)-8-(6-bromo-2-naphthyl)-6-isobutyl-5-oxo-8-(trifluoromethyl)-1-thia-4,7-diazacycloundec-9-yne-3-carbonitrile;
(3R,6S,8R)-8-(6-bromo-2-naphthyl)-6-isobutyl-5-oxo-8-(trifluoromethyl)-1-thia-4,7-diazacycloundec-9-yne-3-carboxamide;
(4S,7S,9R)-9-(4-bromophenyl)-7-isobutyl-2,6-dioxo-9-(trifluoromethyl)-1-oxa-5,8-diazacyclododec-10-yne-4-carboxamide;
(3R,6S,8R)-6-(2-fluoro-2-methylpropyl)-8-[4′-(methylsulfonyl)biphenyl-4-yl]-5-oxo-8-(trifluoromethyl)-1-thia-4,7-diazacycloundec-9-yne-3-carbonitrile;
1-{4′-[(3R,6S,8R)-3-cyano-6-(2-fluoro-2-methylpropyl)-5-oxo-8-(trifluoromethyl)-1-thia-4,7-diazacycloundec-9-yn-8-yl]biphenyl-4-yl}cyclopropanecarboxamide;
(3R,6S,8R)-8-biphenyl-4-yl-6-(2-fluoro-2-methylpropyl)-5-oxo-8-(trifluoromethyl)-1-thia-4,7-diazacycloundec-9-yne-3-carbonitrile;
(3R,6S,8R)-8-(4-bromophenyl)-6-(2-fluoro-2-methylpropyl)-5-oxo-8-(trifluoromethyl)-1-thia-4,7-diazacycloundec-9-yne-3-carbonitrile 1-oxide;
(3R,6S,8R)-8-(4-bromophenyl)-6-(2-fluoro-2-methylpropyl)-5-oxo-8-(trifluoromethyl)-1-thia-4,7-diazacycloundec-9-yne-3-carbonitrile;
(3R,6S,8R)-6-(2-fluoro-2-methylpropyl)-5-oxo-8-(4′-piperazin-4-ium-1-ylbiphenyl-4-yl)-8-(trifluoromethyl)-1-thia-4,7-diazacycloundec-9-yne-3-carboxylate;
(3R,6S,8R)-3-acetyl-8-(4-bromophenyl)-6-(2-methylprop-2-en-1-yl)-8-(trifluoromethyl)-1-thia-4,7-diazacycloundec-9-yn-5-one;
(3R,6S,8R)-8-(4-bromophenyl)-6-(2-fluoro-2-methylpropyl)-N-methoxy-N-methyl-5-oxo-8-(trifluoromethyl)-1-thia-4,7-diazacycloundec-9-yne-3-carboxamide;
(3R,6S,8R)-8-(4-bromophenyl)-6-(2-fluoro-2-methylpropyl)-5-oxo-8-(trifluoromethyl)-1-thia-4,7-diazacycloundec-9-yne-3-carboxylic acid;
(3R,6S,8R)-8-(4-bromophenyl)-6-(2-fluoro-2-methylpropyl)-3-(hydroxymethyl)-8-(trifluoromethyl)-1-thia-4,7-diazacycloundec-9-yn-5-one;
methyl (3R,6S,8R)-8-(4-bromophenyl)-6-(2-fluoro-2-methylpropyl)-5-oxo-8-(trifluoromethyl)-1-thia-4,7-diazacycloundec-9-yne-3-carboxylate;
(3R,6S,8R)-6-(2-fluoro-2-methylpropyl)-8-[4′-(methylsulfonyl)biphenyl-4-yl]-5-oxo-8-(trifluoromethyl)-1-thia-4,7-diazacycloundec-9-yne-3-carboxamide;
(3R,6S,8R)-8-(4-bromophenyl)-6-(2-fluoro-2-methylpropyl)-5-oxo-8-(trifluoromethyl)-1-thia-4,7-diazacycloundec-9-yne-3-carboxamide;
(3R,6S,8R)-6-isobutyl-8-[4′-(methylsulfonyl)biphenyl-4-yl]-5-oxo-8-(trifluoromethyl)-1-thia-4,7-diazacycloundec-9-yne-3-carbonitrile;
1-{4′-[(3R,6S,8R)-3-cyano-6-isobutyl-5-oxo-8-(trifluoromethyl)-1-thia-4,7-diazacycloundec-9-yn-8-yl]biphenyl-4-yl}cyclopropanecarboxamide;
(3R,6S,8R)-8-(4-bromophenyl)-6-isobutyl-5-oxo-8-(trifluoromethyl)-1-thia-4,7-diazacycloundec-9-yne-3-carbonitrile;
(3R,6S,8R)-8-(4-bromophenyl)-6-isobutyl-5-oxo-8-(trifluoromethyl)-1-thia-4,7-diazacycloundec-9-yne-3-carboxamide;
   or a pharmaceutically acceptable salt, stereoisomer or N-oxide derivative thereof.

Also included within the scope of the present invention is a pharmaceutical composition which is comprised of a compound of Formula I as described above and a pharmaceutically acceptable carrier. The invention is also contemplated to encompass a pharmaceutical composition which is comprised of a pharmaceutically acceptable carrier and any of the compounds specifically disclosed in the present application. These and other aspects of the invention will be apparent from the teachings contained herein.

### Utilities

The compounds of the present invention are inhibitors of cathepsins and are therefore useful to treat or prevent cathepsin dependent diseases or conditions in mammals, preferably humans. Specifically, the compounds of the present invention are inhibitors of Cathepsin K and are therefore useful to treat or prevent Cathepsin K dependent diseases or conditions in mammals, preferably humans.

“Cathepsin dependent diseases or conditions” refers to pathologic conditions that depend on the activity of one or more cathepsins. “Cathepsin K dependent diseases or conditions” refers to pathologic conditions that depend on the activity of Cathepsin K. Diseases associated with Cathepsin K activities include osteoporosis, glucocorticoid induced osteoporosis, Paget's disease, abnormally disease, tooth loss, bone fractures, rheumatoid arthritis, osteoarthritis, periprosthetic osteolysis, osteogenesis imperfecta, atherosclerosis, obesity, glaucoma, chronic obstructive pulmonary disease and cancer including metastatic bone disease, hypercalcemia of malignancy, and multiple myeloma. In treating such conditions with the instantly claimed compounds, the required therapeutic amount will vary according to the specific disease and is readily ascertainable by those skilled in the art. Although both treatment and prevention are contemplated by the scope of the invention, the treatment of these conditions is the preferred use.

An embodiment of the invention is a method of inhibiting cathepsin activity in a mammal in need thereof, comprising administering to the mammal a therapeutically effective amount of any of the compounds or any of the pharmaceutical compositions described above.

A class of the embodiment is the method wherein the cathepsin activity is cathepsin K activity.

Another embodiment of the invention is a method of treating or preventing cathepsin dependent conditions in a mammal in need thereof, comprising administering to the mammal a therapeutically effective amount of any of the compounds or any of the pharmaceutical compositions described above.

A class of the embodiment is the method wherein the cathepsin activity is cathepsin K activity.

Another embodiment of the invention is a method of inhibiting bone loss in a mammal in need thereof, comprising administering to the mammal a therapeutically effective amount of any of the compounds or any of the pharmaceutical compositions described above. Another embodiment of the invention is a method of reducing bone loss in a mammal in need thereof, comprising administering to the mammal a therapeutically effective amount of any of the compounds or any of the pharmaceutical compositions described above. The utility of cathepsin K inhibitors in the inhibition of bone resorption, which includes abnormally increased bone turnover, bone fractures, Paget's disease, osteogenesis imperfecta and periprosthetic osteolysis, is known in the literature, see Stroup, G. B., Lark, M. W., Veber, D F., Bhattacharrya, A., Blake, S., Dare, L. C., Erhard, K. F., Hoffman, S. J., James, I. E., Marquis, R.w., Ru, Y., Vasko-Moser, J. A., Smith, B. R., Tomaszek, T. and Gowen, M. Potent and selective inhibition of human cathepsin K leads to inhibition of bone resorption in vivo in a nonhuman primate. J. Bone Miner. Res., 16:1739-1746; 2001; and Votta, B. J., Levy, M. A., Badger, A., Dodds, R. A., James, I. E., Thompson, S., Bossard, M. J., Can, T., Connor, J. R., Tomaszek, T. A., Szewczuk, L., Drake, F. H., Veber, D., and Gowen, M. Peptide aldehyde inhibitors of cathepsin K inhibit bone resorption both in vivo and in vitro. J. Bone Miner. Res. 12:1396-1406; 1997.

Another embodiment of the invention is a method of treating or preventing osteoporosis, including glucocorticoid induced osteoporosis, in a mammal in need thereof, comprising administering to the mammal a therapeutically effective amount of any of the compounds or any of the above pharmaceutical compositions described above. The utility of cathepsin K inhibitors in the treatment or prevention of osteoporosis is known in the literature, see Saftig, P., Hunziker, E., Wehmeyer, O., Jones, S., Boyde, A., Rommerskirch, W., Moritz, J. D., Schu, P., and Vonfigura, K. Impaired osteoclast bone resorption leads to osteopetrosis in cathepsin K-deficient mice. Proc. Natl. Acad. Sci. USA 95:13453-13458; 1998.

Another embodiment of the invention is a method of treating or preventing periodontal disease, including tooth loss, in a mammal in need thereof, comprising administering to the mammal a therapeutically effective amount of any of the compounds or any of the above pharmaceutical compositions described above. The utility of cathepsin K inhibitors in the treatment or prevention of periodontal disease and tooth loss is known in the literature, see Tsuji Y, et al., Expression of cathepsin K mRNA and protein in odontoclasts after experimental tooth movement in the mouse maxilla by in situ hybridization and immunoelectron microscopy. Cell Tissue Res. 2001 March; 303(3):359-69.

Another embodiment of the invention is a method of treating or preventing rheumatoid arthritic condition in a mammal in need thereof, comprising administering to the mammal a therapeutically effective amount of any of the compounds or any of the pharmaceutical compositions described above. It is known in the literature that progressive destruction of the periarticular bone is a major cause of joint dysfunction and disability in patients with rheumatoid arthritis (RA), see Goldring S R, “Pathogenesis of bone erosions in rheumatoid arthritis”. Curr. Opin. Rheumatol. 2002; 14: 406-10. Analysis of joint tissues from patients with RA have provided evidence that cathepsin K positive osteoclasts are the cell types that mediate the focal bone resorption associated with rheumatoid synovial lesion, see Hou, W-S, Li, W, Keyszer, G, Weber, E, Levy, R, Klein, M J, Gravallese, E M, Goldring, S R, Bromme, D, “Comparison of Cathepsin K and S expression within the Rheumatoid and Osteoarthritic Synovium”, Arthritis Rheumatism 2002; 46: 663-74. In addition, generalized bone loss is a major cause of morbidity associated with severe RA. The frequency of hip and spinal fractures is substantially increased in patients with chronic RA, see Gould A, Sambrook, P, Devlin J et al, “Osteoclastic activation is the principal mechanism leading to secondary osteoporosis in rheumatoid arthritis”. J. Rheumatol. 1998; 25: 1282-9. The utility of cathepsin K inhibitors in the treatment or prevention of resorption in subarticular bone and of generalized bone loss represent a rational approach for pharmacological intervention on the progression of rheumatoid arthritis.

Another embodiment of the invention is a method of treating or preventing the progression of osteoarthritis in a mammal in need thereof, comprising administering to the mammal a therapeutically effective amount of any of the compounds or any of the pharmaceutical compositions described above. It is known in the literature that osteoarthritis (OA) is accompanied with well-defined changes in the joints, including erosion of the articular cartilage surface, peri-articular endochondral ossification/osteophytosis, and subchondral bony sclerosis and cyst formation, see Oettmeier R, Abendroth, K, “Osteoarthritis and bone: osteologic types of osteoarthritis of the hip”, Skeletal Radiol. 1989; 18: 165-74. Recently, the potential contribution of subchondral bone sclerosis to the initiation and progression of OA have been suggested. Stiffened subchondral bone as the joint responding to repetitive impulsive loading, is less able to attenuate and distribute forces through the joint, subjecting it to greater mechanical stress across the articular cartilage surface. This in turn accelerates cartilage wear and fibrillate, see Radin, E L and Rose R M, “Role of subchondral bone in the initiation and progression of cartilage damage”, Clin. Orthop. 1986; 213: 34-40. Inhibition of excessive subarticular bone resorption by an anti-resorptive agent such as a cathepsin K inhibitor, will lead to inhibition of subchondral bone turnover, thus may have a favorable impact on OA progression.

In addition to the above hypothesis, cathepsin K protein expression was recently identified in synovial fibroblasts, macrophage-like cells, and chondrocytes from synovium and articular cartilage specimens derived from OA patients, see Hou, W-S, Li, W, Keyszer, G, Weber, E, Levy, R, Klein, M J, Gravallese, E M, Goldring, S R, Bromme, D, “Comparison of Cathepsin K and S expression within the Rheumatoid and Osteoarthritic Synovium”, Arthritis Rheumatism 2002; 46: 663-74; and Dodd, R A, Connor, J R, Drake, F H, Gowen, M, “Expression of Cathepsin K messenger RNA in giant cells and their precursors in human osteoarthritic synovial tissues”. Arthritis Rheumatism 1999; 42: 1588-93; and Konttinen, Y T, Mandelin, J, Li, T-F, Salo, J, Lassus, J et al. “Acidic cysteine endoproteinase cathepsin K in the degeneration of the superficial articular hyaline cartilage in osteoarthritis”, Arthritis Rheumatism 2002; 46: 953-60. These recent studies thus implicated the role of cathepsin K in the destruction of collagen type II in the articular cartilage associated with the progression of osteoarthritis. The utility of cathepsin K inhibitors in the treatment or prevention of osteoarthritis as described in this invention thus comprise of two different mechanisms, one is on the inhibition of osteoclast-driven subchondral bone turnover, and two is on the direct inhibition of collagen type II degeneration in the synovium and cartilage of patients with OA.

Another embodiment of the invention is a method of treating cancer in a mammal in need thereof, comprising administering to the mammal a therapeutically effective amount of any of the compounds or any of the pharmaceutical compositions described above. It is known in the literature that cathepsin K is expressed in human breast carcinoma, prostate cancer and chordoma and has matrix degrading capabilities, see Littlewood-Evans A J, Bilbe G, Bowler W B, Farley D, Wlodarski B, Kokubo T, Inaoka T, Sloane J, Evans D B, Gallagher J A, “The osteoclast-associated protease cathepsin K is expressed in human breast carcinoma.” Cancer Res 1997 Dec. 1; 57(23):5386-90, Brubaker K D, Vessella R L, True L D, Thomas R, Corey E “Cathepsin K mRNA and protein expression in prostate cancer progression.” J Bone Miner Res 2003 18, 222-30, Haeckel C, Krueger S, Kuester D, Ostertag H, Samii M, Buehling F, Broemme D, Czerniak B, Roessner A. “Expression of cathepsin K in chordoma.” Hum Pathol 2000 July; 31(7):834-40.

Another embodiment of the invention is a method of treating atherosclerosis in a mammal in need thereof, comprising administering to the mammal a therapeutically effective amount of any of the compounds or any of the pharmaceutical compositions described above. It is known in the literature that cathepsin K is expressed in human atheroma and has significant elastase activity, see Sukhova G K, Shi G P, Simon D I, Chapman H A, Libby P. “Expression of the elastolytic cathepsins S and K in human atheroma and regulation of their production in smooth muscle cells.” J Clin Invest 1998 August 102, 576-83. It is also known that the Cat K null mouse when crossed with an ApoE null mouse shows reduced atherosclerotic plaque area and increased resistance to plaque rupture, see E. Lutgens, S. P. M. Lutgens, B. C. G. Faber, S. Heeneman, M. M. J. Gijbels, M. P. J. de Winther, P. Frederik, I. van der Made, D. Black, M. J. A. P. Daemen, K. B. J. M. Cleutjens “Disruption of the Cathepsin K Gene Reduces Atherosclerosis Progression and Induces Plaque Fibrosis but Accelerates Macrophage Foam Cell Formation.” Circulation 2006 113:98-107. Increased plaque stability would lead to a decrease in heart attack and stroke in a patient administered a therapeutically effective around of any of the compounds or any of the pharmaceutical compositions described above.

Another embodiment of the invention is a method of treating obesity in a mammal in need thereof, comprising administering to the mammal a therapeutically effective amount of any of the compounds or any of the pharmaceutical compositions described above. It is known in the literature that cathepsin K mRNA is increased in adipose tissue in several mouse models of obesity and also in adipose tissue of obese human males, see Chiellini C, Costa M, Novelli S E, Amri E Z, Benzi L, Bertacca A, Cohen P, Del Prato S, Friedman J M, Maffei M. “Identification of cathepsin K as a novel marker of adiposity in white adipose tissue,” J Cell Physiol 2003, 195, 309-21.

Another embodiment of the invention is a method of treating glaucoma in a mammal in need thereof, comprising administering to the mammal a therapeutically effective amound of any of the compounds or any of the pharmaceutical compositions described above. Cathepsin K is highly expressed in the iris, ciliary body and retinal pigment epithelium, and as such can be useful in the treatment of glaucoma, see Ortega, J., et al., “Gene Expression of Proteases and Protease Inhibitors in the Human Ciliary Epithelium and ODM-2 cells,” Exp. Eye Res (1997) 65, 289-299; International Publication WO 2004/058238 (Alcon, Inc.).

Another embodiment of the invention is a method of treating chronic obstructive pulmonary disease in a mammal in need thereof, comprising administering to the mammal a therapeutically effective amount of any of the compounds or any of the pharmaceutical compositions described above. It is known in the literature that cathepsin K plays a role in lung fibrosis, see Buhling, F., et al., “Pivotal role of cathepsin K in lung fibrosis,” Am J Pathol. 2004 June; 164(6):2203-16.

Another embodiment of the invention is a method of treating parasitic infections in a mammal in need thereof, comprising administering to the mammal a therapeutically effective amount of any of the compounds or any of the pharmaceutical compositions described above. It is known in the literature that mammalian cathepsins are related to the papain-like cysteine proteases which play an important role in the life cycle of these parasites. Such parasites are involved in the diseases of malaria, American trypanosomiasis, African trypanosomiasis, leishmaniasis, giardiasis, trichomoniasis, amoebiasis, schistosomiasis, fascioliasis, paragonimiasis and intestinal roundworms, see Lecaille F, Kaleta J, Bromme D., Human and parasitic papain-like cysteine proteases: their role in physiology and pathology and recent developments in inhibitor design. Chem Rev 2002 102, 4459-88.

Another embodiment of the invention is a method of treating severe acute respiratory syndrome (SARS) in a mammal in need thereof, comprising administering to the mammal a therapeutically effective amount of any of the compounds or any of the pharmaceutical compositions described above.

Another embodiment of the invention is a method of treating metastatic bone disease in a mammal in need thereof, comprising administering to the mammal a therapeutically effective amount of any of the compounds or any of the pharmaceutical compositions described above. It is known in the literature that osteoclasts are responsible for bone resorption and that bone destruction and hypercalcemia induced by metastatic tumors are carried out by osteoclasts. Accordingly, the inhibition of osteoclasts can prevent bone destruction and bone metastasis, see Miyamoto, T. and Suda, T., “Differentiation and function of osteoclasts,” Keio J Med 2003 March; 52(1):1-7.

Another embodiment of the invention is a method of preventing metastatic bone disease in a mammal with a primary tumor that carries a risk of bone metastasis, comprising administering to the mammal a therapeutically effective amount of any of the compounds or any of the pharmaceutical compositions described above. It is described in the literature that compounds that inhibit osteoclasts function can prevent tumor cell adhesion to bone, see S. Boissier, M. Ferreras, O. Peyruchaud, S. Magnetto, F. H. Ebetino, M. Colombel, P. Delmas, J.-M. Delaisse and P. Clezardin “Bisphosphonates Inhibit Breast and Prostate Carcinoma Cell Invasion, an Early Event in the Formation of Bone Metastases” Cancer Research 60, 2949-2954, 2000

Another embodiment of the invention is a method of treating hypercalcemia of malignancy or multiple myeloma in a mammal in need thereof, comprising administering to the mammal a therapeutically effective amount of any of the compounds or any of the pharmaceutical compositions described above. It is known in the literature that cathepsin K plays a role in hypercalcemia of malignancy and multiple myeloma, see Faust, J. et al., Multiple myeloma cells and cells of the human osteoclast lineage share morphological and cell surface markers. J Cell Biochem. 1998 Dec. 15; 71(4):559-68; A. lipton, New therapeutic agents for the treatment of bone diseases. Expert Opin Biol Ther. 2005 June; 5(6):817-32.

Another embodiment of the invention is administering to a mammal a therapeutically effective amount of any of the compounds or any of the pharmaceutical compositions described above for the treatment of mammalian diseases associated with cathepsin S including Alzheimer's disease, atherosclerosis, neuropathic and inflammatory pain, obesity, diabetes, chronic obstructive pulmonary disease, cancer and certain autoimmune disorders, including, but not limited to juvenile onset diabetes, multiple sclerosis, pemphigus vulgaris, Graves' disease, myasthenia gravis, systemic lupus erythemotasus, rheumatoid arthritis and Hashimoto's thyroiditis; allergic disorders, including, but not limited to asthma; and allogenic immune responses, including, but not limited to, rejection of organ transplants or tissue grafts. It is known in the literature that cathepsin S activity is associated with the above disease states, see Munger J S, Haass C, Lernere C A, Shi G P, Wong W S, Teplow D B, Selkoe D J, Chapman H A. Lysosomal processing of amyloid precursor protein to A beta peptides: a distinct role for cathepsin S. Biochem J 1995 311, 299-305, Sukhova G K, Zhang Y, Pan J H, Wada Y, Yamamoto T, Naito M, Kodama T, Tsimikas S, Witztum J L, Lu M L, Sakara Y, Chin M T, Libby P, Shi G P. Deficiency of cathepsin S reduces atherosclerosis in LDL receptor-deficient mice. J Clin Invest 2003 111, 897-906, Zheng T, Zhu Z, Wang Z, Horner R J, Ma B, Riese R J Jr, Chapman H A Jr, Shapiro S D, Elias J A. Inducible targeting of IL-13 to the adult lung causes matrix metalloproteinase- and cathepsin-dependent emphysema. J Clin Invest 2000 106, 1081-93, Shi G P, Sukhova G K, Kuzuya M, Ye Q, Du J, Zhang Y, Pan J H, Lu M L, Cheng X W, Iguchi A, Perrey S, Lee A M, Chapman H A, Libby P. Deficiency of the cysteine protease cathepsin S impairs microvessel growth. Circ Res 2003 92, 493-500, Nakagawa T Y, Brissette W H, Lira P D, Griffiths R J, Petrushova N, Stock J, McNeish J D, Eastman S E, Howard E D, Clarke S R, Rosloniec E F, Elliott E A, Rudensky A Y. Impaired invariant chain degradation and antigen presentation and diminished collagen-induced arthritis in cathepsin S null mice Immunity 1999 10, 207-17. Barclay J, Role of the cysteine protease cathepsin S in neuropathic hyperalgesia. Pain. 2007 in press. Taleb S et al, FASEB J. 2005 September; 19(11):1540-2. Cathepsin S, a novel biomarker of adiposity: relevance to atherogenesis.

Another embodiment of the invention is administering to a mammal a therapeutically effective amount of any of the compounds or any of the pharmaceutical compositions described above for the treatment of mammalian diseases associated with cathepsin B. Increased levels of cathepsin B and redistribution of the enzyme are found in tumours, suggesting a role for cathepsin B in tumor invasion and metastasis. In addition, aberrant cathepsin B activity is implicated in rheumatoid arthritis, osteoarthritis, pneumocystis carinii, acute pancreatitis, inflammatory airway disease and bone and joint disorders. Inhibitors of cathepsin B and/or cathepsin S have been recommended for use in treating chronic obstructive pulmonary disease (COPD) (WO 2004/089395).

Furthermore, recent studies suggest that cathepsin B plays a pivotal role in Alzheimer's disease and other dementing conditions.

Alzheimer's disease (AD) is the most prevalent form of dementia. Its diagnosis is described in the Diagnostic and Statistical Manual of Mental Disorders, 4^{th} ed., published by the American Psychiatric Association (DSM-IV). It is a neurodegenerative disorder, clinically characterized by progressive loss of memory and general cognitive function, and pathologically characterized by the deposition of extracellular proteinaceous plaques in the cortical and associative brain regions of sufferers. These plaques mainly comprise fibrillar aggregates of β-amyloid peptide (Aβ). Aβ is formed from amyloid precursor protein (APP) via separate intracellular proteolytic events involving the enzymes β-secretase and γ-secretase. Variability in the site of the proteolysis mediated by γ-secretase results in Aβ of varying chain length, e.g. Aβ(1-38), Aβ(1-40) and Aβ(1-42). N-terminal truncations such as Aβ(4-42) are also found in the brain, possibly as a result of variability in the site of proteolysis mediated by β-secretase. For the sake of convenience, expressions such as “Aβ(1-40)” and “Aβ(1-42)” as used herein are inclusive of such N-terminal truncated variants. After secretion into the extracellular medium, Aβ forms initially-soluble aggregates which are widely believed to be the key neurotoxic agents in AD (see Gong et al, PNAS, 100 (2003), 10417-22), and which ultimately result in the insoluble deposits and dense neuritic plaques which are the pathological characteristics of AD.

Other dementing conditions associated with deposition of Aβ in the brain include cerebral amyloid angiopathy, hereditary cerebral haemorrhage with amyloidosis, Dutchtype (HCHWA-D), multi-infarct dementia, dementia pugilistica and Down syndrome.

Hook et. Al. (J. Neurochem., 2002, 81, 237-56) identified two distinct pathways leading to secretion of Aβ, namely a regulated secretory pathway and a constitutive secretory pathway, and showed that different β-secretase enzymes were involved in these distinct pathways. Later work by the same group (Hook et. al., Biol. Chem., 2005, 386, 931-40) showed that cathepsin B acts as β-secretase in the regulated pathway, which is the major source of secreted extracellular Aβ. Hence, inhibitors of cathepsin B, in particular selective inhibitors, are of great interest as a potential treatment of AD. See, Seyfried D M et al, A selective cysteine protease inhibitor is non-toxic and cerebroprotective in rats undergoing transient middle cerebral artery ischemia, Brain Res. 2001 May 18; 901(1-2):94-101.

Inhibitors of cathepsin B have also been linked to treat inflammatory bowel diseases, see Menzel K et al, Clin Exp Immunol. 2006 October; 146(1):169-80. Cathepsins B, L and D in inflammatory bowel disease macrophages and potential therapeutic effects of cathepsin inhibition in vivo.

Antoher embodiment of the invention is the treatment of liver disease. It is known in the art that inhibitors of cathepsin B can play a role in the treatment of liver disease, see Canbay A., et al., Cathepsin B inactivation attenuates hepatic injury and fibrosis during cholestasis,

### J Clin Invest. 2003 July; 112(2):152-9.

Another embodiment of the invention is the treatment or prevention of stroke. It is known in the art that cathepsins B and L can be useful for the treatment or prevention of stroke, see Seyfried D M et al, A selective cysteine protease inhibitor is non-toxic and cerebroprotective in rats undergoing transient middle cerebral artery ischemia. Brain Res. 2001 May 18; 901(1-2):94-101.

Exemplifying the invention is the use of any of the compounds described above in the preparation of a medicament for the treatment and/or prevention of osteoporosis in a mammal in need thereof. Still further exemplifying the invention is the use of any of the compounds described above in the preparation of a medicament for the treatment and/or prevention of: bone loss, bone resorption, bone fractures, metastatic bone disease and/or disorders related to cathepsin functioning.

The compounds of this invention may be administered to mammals, preferably humans, either alone or, preferably, in combination with pharmaceutically acceptable carriers or diluents, optionally with known adjuvants, such as alum, in a pharmaceutical composition, according to standard pharmaceutical practice. The compounds can be administered orally or parenterally, including the intravenous, intramuscular, intraperitoneal, subcutaneous, rectal and topical routes of administration.

In the case of tablets for oral use, carriers which are commonly used include lactose and corn starch, and lubricating agents, such as magnesium stearate, are commonly added. For oral administration in capsule form, useful diluents include lactose and dried corn starch. For oral use of a therapeutic compound according to this invention, the selected compound may be administered, for example, in the form of tablets or capsules, or as an aqueous solution or suspension. For oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. When aqueous suspensions are required for oral use, the active ingredient is combined with emulsifying and suspending agents. If desired, certain sweetening and/or flavoring agents may be added. For intramuscular, intraperitoneal, subcutaneous and intravenous use, sterile solutions of the active ingredient are usually prepared, and the pH of the solutions should be suitably adjusted and buffered. For intravenous use, the total concentration of solutes should be controlled in order to render the preparation isotonic.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polyactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

The instant compounds are also useful in combination with known agents useful for treating or preventing osteoporosis, glucocorticoid induced osteoporosis, Paget's disease, abnormally increased bone turnover, periodontal disease, tooth loss, bone fractures, rheumatoid arthritis, osteoarthritis, periprosthetic osteolysis, osteogenesis imperfecta, metastatic bone disease, hypercalcemia of malignancy, and multiple myeloma. Combinations of the presently disclosed compounds with other agents useful in treating or preventing osteoporosis or other bone disorders are within the scope of the invention. A person of ordinary skill in the art would be able to discern which combinations of agents would be useful based on the particular characteristics of the drugs and the disease involved. Such agents include the following: an organic bisphosphonate; a selective estrogen receptor modulator; an androgen receptor modulator; an inhibitor of osteoclast proton ATPase; an inhibitor of HMG-CoA reductase; an integrin receptor antagonist; an osteoblast anabolic agent, such as PTH; Vitamin D; a synthetic Vitamin D analogue; a Nonsteroidal anti-inflammatory drug; a selective cyclooxygenase-2 inhibitor; an inhibitor of interleukin-1 beta; a LOX/COX inhibitor; and the pharmaceutically acceptable salts and mixtures thereof. A preferred combination is a compound of the present invention and an organic bisphosphonate. Another preferred combination is a compound of the present invention and a selective estrogen receptor modulator. Another preferred combination is a compound of the present invention and an androgen receptor modulator. Another preferred combination is a compound of the present invention and an osteoblast anabolic agent.

“Organic bisphosphonate” includes, but is not limited to, compounds of the

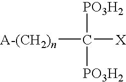

wherein n is an integer from 0 to 7 and wherein A and X are independently selected from the group consisting of H, OH, halogen, NH₂, SH, phenyl, C₁-C₃₀ alkyl, C₃-C₃₀ branched or cycloalkyl, bicyclic ring structure containing two or three N, C₁-C₃₀ substituted alkyl, C₁-C₁₀ alkyl substituted NH₂, C₃-C₁₀ branched or cycloalkyl substituted NH₂, C₁-C₁₀ dialkyl substituted NH₂, C₁-C₁₀ alkoxy, C₁-C₁₀ alkyl substituted thio, thiophenyl, halophenylthio, C₁-C₁₀ alkyl substituted phenyl, pyridyl, furanyl, pyrrdidinyl, imidazolyl, imidazopyridinyl, and benzyl, such that both A and X are not selected from H or OH when n is 0; or A and X are taken together with the carbon atom or atoms to which they are attached to form a C₃-C₁₀ ring.

In the foregoing chemical formula, the alkyl groups can be straight, branched, or cyclic, provided sufficient atoms are selected for the chemical formula. The C₁-C₃₀ substituted alkyl can include a wide variety of substituents, nonlimiting examples which include those selected from the group consisting of phenyl, pyridyl, furanyl, pyrrolidinyl, imidazonyl, NH₂, C₁-C₁₀ alkyl or dialkyl substituted NH₂, OH, SH, and C₁-C₁₀ alkoxy.

The foregoing chemical formula is also intended to encompass complex carbocyclic, aromatic and hetero atom structures for the A and/or X substituents, nonlimiting examples of which include naphthyl, quinolyl, isoquinolyl, adamantyl, and chlorophenylthio.

Pharmaceutically acceptable salts and derivatives of the bisphosphonates are also useful herein. Non-limiting examples of salts include those selected from the group consisting alkali metal, alkaline metal, ammonium, and mono-, di-, tri-, or tetra-C₁-C₁₀-alkyl-substituted ammonium. Preferred salts are those selected from the group consisting of sodium, potassium, calcium, magnesium, and ammonium salts. More preferred are sodium salts. Non-limiting examples of derivatives include those selected from the group consisting of esters, hydrates, and amides.

It should be noted that the terms “bisphosphonate” and “bisphosphonates”, as used herein in referring to the therapeutic agents of the present invention are meant to also encompass diphosphonates, biphosphonic acids, and diphosphonic acids, as well as salts and derivatives of these materials. The use of a specific nomenclature in referring to the bisphosphonate or bisphosphonates is not meant to limit the scope of the present invention, unless specifically indicated. Because of the mixed nomenclature currently in use by those of ordinary skill in the art, reference to a specific weight or percentage of a bisphosphonate compound in the present invention is on an acid active weight basis, unless indicated otherwise herein. For example, the phrase “about 5 mg of a bone resorption inhibiting bisphosphonate selected from the group consisting of alendronate, pharmaceutically acceptable salts thereof, and mixtures thereof, on an alendronic acid active weight basis” means that the amount of the bisphosphonate compound selected is calculated based on 5 mg of alendronic acid.

Non-limiting examples of bisphosphonates useful herein include the following:

Alendronate, which is also known as alendronic acid, 4-amino-1-hydroxybutylidene-1,1-bisphosphonic acid, alendronate sodium or alendronate monosodium trihydrate, 4-amino-1-hydroxybutylidene-1,1-bisphosphonic acid monosodium trihydrate.

Alendronate is described in U.S. Pat. Nos. 4,922,007, to Kieczykowski et al., issued May 1, 1990; 5,019,651, to Kieczykowski et al., issued May 28, 1991; 5,510,517, to Dauer et al., issued Apr. 23, 1996; 5,648,491, to Dauer et al., issued Jul. 15, 1997, all of which are incorporated by reference herein in their entirety.

Cycloheptylaminomethylene-1,1-bisphosphonic acid, YM 175, Yamanouchi (incadronate, formerly known as cimadronate), as described in U.S. Pat. No. 4,970,335, to Isomura et al., issued Nov. 13, 1990, which is incorporated by reference herein in its entirety.

1,1-dichloromethylene-1,1-diphosphonic acid (clodronic acid), and the disodium salt (clodronate, Procter and Gamble), are described in Belgium Patent 672,205 (1966) and J. Org. Chem. 32, 4111 (1967), both of which are incorporated by reference herein in their entirety.

1-hydroxy-3-(1-pyrrolidinyl)-propylidene-1,1-bisphosphonic acid (EB-1053).

1-hydroxyethane-1,1-diphosphonic acid (etidronic acid).

1-hydroxy-3-(N-methyl-N-pentylamino)propylidene-1,1-bisphosphonic acid, also known as BM-210955, Boehringer-Mannheim (ibandronate), is described in U.S. Pat. No. 4,927,814, issued May 22, 1990, which is incorporated by reference herein in its entirety.

1-hydroxy-2-imidazo-(1,2-a)pyridin-3-yethylidene (minodronate).

6-amino-1-hydroxyhexylidene-1,1-bisphosphonic acid (neridronate).

3-(dimethylamino)-1-hydroxypropylidene-1,1-bisphosphonic acid (olpadronate).

3-amino-1-hydroxypropylidene-1,1-bisphosphonic acid (pamidronate).

[2-(2-pyridinypethylidene]-1,1-bisphosphonic acid (piridronate) is described in U.S. Pat. No. 4,761,406, which is incorporated by reference in its entirety.

1-hydroxy-2-(3-pyridinyl)-ethylidene-1,1-bisphosphonic acid (risedronate).

(4-chlorophenyl)thiomethane-1,1-disphosphonic acid (tiludronate) as described in U.S. Pat. No. 4,876,248, to Breliere et al., Oct. 24, 1989, which is incorporated by reference herein in its entirety.

1-hydroxy-2-(1H-imidazol-1-yl)ethylidene-1,1-bisphosphonic acid (zoledronate).

Nonlimiting examples of bisphosphonates include alendronate, cimadronate, clodronate, etidronate, ibandronate, incadronate, minodronate, neridronate, olpadronate, pamidronate, piridronate, risedronate, tiludronate, and zolendronate, and pharmaceutically acceptable salts and esters thereof. A particularly preferred bisphosphonate is alendronate, especially a sodium, potassium, calcium, magnesium or ammonium salt of alendronic acid. Exemplifying the preferred bisphosphonate is a sodium salt of alendronic acid, especially a hydrated sodium salt of alendronic acid. The salt can be hydrated with a whole number of moles of water or non whole numbers of moles of water. Further exemplifying the preferred bisphosphonate is a hydrated sodium salt of alendronic acid, especially when the hydrated salt is alendronate monosodium trihydrate.

It is recognized that mixtures of two or more of the bisphosphonate actives can be utilized.

The precise dosage of the organic bisphosphonate will vary with the dosing schedule, the particular bisphosphonate chosen, the age, size, sex and condition of the mammal or human, the nature and severity of the disorder to be treated, and other relevant medical and physical factors. Thus, a precise pharmaceutically effective amount cannot be specified in advance and can be readily determined by the caregiver or clinician. Appropriate amounts can be determined by routine experimentation from animal models and human clinical studies. Generally, an appropriate amount of bisphosphonate is chosen to obtain a bone resorption inhibiting effect, i.e. a bone resorption inhibiting amount of the bisphosphonate is administered. For humans, an effective oral dose of bisphosphonate is typically from about 1.5 to about 6000 μg/kg body weight and preferably about 10 to about 2000 μg/kg of body weight. For alendronate monosodium trihydrate, common human doses which are administered are generally in the range of about 2 mg/day to about 40 mg/day, preferably about 5 mg/day to about 40 mg/day. In the U.S. presently approved dosages for alendronate monosodium trihydrate are 5 mg/day for preventing osteoporosis, 10 mg/day for treating osteoporosis, and 40 mg/day for treating Paget's disease.

In alternative dosing regimens, the bisphosphonate can be administered at intervals other than daily, for example once-weekly dosing, twice-weekly dosing, biweekly dosing, and twice-monthly dosing. In a once weekly dosing regimen, alendronate monosodium trihydrate would be administered at dosages of 35 mg/week or 70 mg/week.

“Selective estrogen receptor modulators” refers to compounds which interfere or inhibit the binding of estrogen to the receptor, regardless of mechanism. Examples of estrogen receptor modulators include, but are not limited to, estrogen, progestogen, estradiol, droloxifene, raloxifene, lasofoxifene, TSE-424, tamoxifen, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

An “estrogen receptor beta modulator” is a compound that selectively agonizes or antagonizes estrogen receptor beta (ERβ Agonizing ERβ increases transcription of the tryptophan hydroxylase gene (TPH, the key enzyme in serotonin synthesis) via an ERβ mediated event. Examples of estrogen receptor beta agonists can be found in PCT International publication WO 01/82923, which published on Nov. 8, 2001, and WO 02/41835, which published on May 20, 2002, both of which are hereby incorporated by reference in their entirety.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“An inhibitor of osteoclast proton ATPase” refers to an inhibitor of the proton ATPase, which is found on the apical membrane of the osteoclast, and has been reported to play a significant role in the bone resorption process. This proton pump represents an attractive target for the design of inhibitors of bone resorption which are potentially useful for the treatment and prevention of osteoporosis and related metabolic diseases. See C. Farina et al., “Selective inhibitors of the osteoclast vacuolar proton ATPase as novel bone antiresorptive agents,” DDT, 4: 163-172 (1999)), which is hereby incorporated by reference in its entirety.

“HMG-CoA reductase inhibitors” refers to inhibitors of 3-hydroxy-3-methylglutaryl-CoA reductase. Compounds which have inhibitory activity for HMG-CoA reductase can be readily identified by using assays well-known in the art. For example, see the assays described or cited in U.S. Pat. No. 4,231,938 at col. 6, and WO 84/02131 at pp. 30-33. The terms “HMG-CoA reductase inhibitor” and “inhibitor of HMG-CoA reductase” have the same meaning when used herein.

Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896), atorvastatin (LIPITOR®; see U.S. Pat. Nos. 5,273,995, 4,681,893, 5,489,691 and 5,342,952) and cerivastatin (also known as rivastatin and BAYCHOL®; see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention. An illustration of the lactone portion and its corresponding open-acid form is shown below as structures I and II.

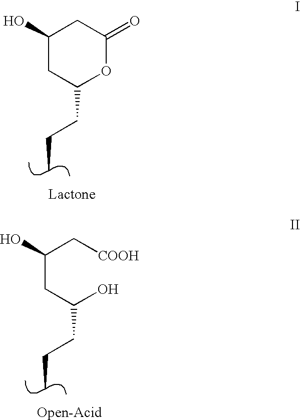

In HMG-CoA reductase inhibitors where an open-acid form can exist, salt and ester forms may preferably be formed from the open-acid, and all such forms are included within the meaning of the term “HMG-CoA reductase inhibitor” as used herein. Preferably, the HMG-CoA reductase inhibitor is selected from lovastatin and simvastatin, and most preferably simvastatin. Herein, the term “pharmaceutically acceptable salts” with respect to the HMG-CoA reductase inhibitor shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenz-imidazole, diethylamine, piperazine, and tris(hydroxymethyl)aminomethane. Further examples of salt forms of HMG-CoA reductase inhibitors may include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate.

Ester derivatives of the described HMG-CoA reductase inhibitor compounds may act as prodrugs which, when absorbed into the bloodstream of a warm-blooded animal, may cleave in such a manner as to release the drug form and permit the drug to afford improved therapeutic efficacy.

As used above, “integrin receptor antagonists” refers to compounds which selectively antagonize, inhibit or counteract binding of a physiological ligand to the αᵥβ₃ integrin, to compounds which selectively antagonize, inhibit or counteract binding of a physiological ligand to the αᵥβ₅ integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₆, αᵥβ₈, α₁β₁, α₂β₁, α₅β₁, α₆β₁ and α₆β₄ integrins. H. N. Lode and coworkers in PNAS USA 96: 1591-1596 (1999) have observed synergistic effects between an antiangiogenic αv integrin antagonist and a tumor-specific antibody-cytokine (interleukin-2) fusion protein in the eradication of spontaneous tumor metastases. Their results suggested this combination as having potential for the treatment of cancer and metastatic tumor growth. αᵥβ₃ integrin receptor antagonists inhibit bone resorption through a new mechanism distinct from that of all currently available drugs. Integrins are heterodimeric transmembrane adhesion receptors that mediate cell-cell and cell-matrix interactions. The α and β integrin subunits interact non-covalently and bind extracellular matrix ligands in a divalent cation-dependent manner. The most abundant integrin on osteoclasts is αᵥβ₃ (>10⁷/osteoclast), which appears to play a rate-limiting role in cytoskeletal organization important for cell migration and polarization. The αᵥβ₃ antagonizing effect is selected from inhibition of bone resorption, inhibition of restenosis, inhibition of macular degeneration, inhibition of arthritis, and inhibition of cancer and metastatic growth.

“An osteoblast anabolic agent” refers to agents that build bone, such as PTH. The intermittent administration of parathyroid hormone (PTH) or its amino-terminal fragments and analogues have been shown to prevent, arrest, partially reverse bone loss and stimulate bone formation in animals and humans. For a discussion refer to D. W. Dempster et al., “Anabolic actions of parathyroid hormone on bone,” Endocr Rev 14: 690-709 (1993). Studies have demonstrated the clinical benefits of parathyroid hormone in stimulating bone formation and thereby increasing bone mass and strength. Results were reported by R M Neer et al., in New Eng J Med 344 1434-1441 (2001).

In addition, parathyroid hormone-related protein fragments or analogues, such as PTHrP-(1-36) have demonstrated potent anticalciuric effects [see M. A. Syed et al., “Parathyroid hormone-related protein-(1-36) stimulates renal tubular calcium reabsorption in normal human volunteers: implications for the pathogenesis of humoral hypercalcemia of malignancy,” JCEM 86: 1525-1531 (2001)] and may also have potential as anabolic agents for treating osteoporosis.

“Vitamin D” includes, but is not limited to, vitamin D₃ (cholecalciferol) and vitamin D₂ (ergocalciferol), which are naturally occurring, biologically inactive precursors of the hydroxylated biologically active metabolites of vitamin D: 1α-hydroxy vitamin D; 25-hydroxy vitamin D, and 1α,25-dihydroxy vitamin D. Vitamin D₂ and vitamin D₃ have the same biological efficacy in humans. When either vitamin D₂ or D₃ enters the circulation, it is hydroxylated by cytochrome P₄₅₀-vitamin D-25-hydroxylase to give 25-hydroxy vitamin D. The 25-hydroxy vitamin D metabolite is biologically inert and is further hydroxylated in the kidney by cytochrome P450-monooxygenase, 25 (OH) D-1α-hydroxylase to give 1,25-dihydroxy vitamin D. When serum calcium decreases, there is an increase in the production of parathyroid hormone (PTH), which regulates calcium homeostasis and increases plasma calcium levels by increasing the conversion of 25-hydroxy vitamin D to 1,25-dihydroxy vitamin D.

1,25-dihydroxy vitamin D is thought to be reponsible for the effects of vitamin D on calcium and bone metabolism. The 1,25-dihydroxy metabolite is the active hormone required to maintain calcium absorption and skeletal integrity. Calcium homeostasis is maintained by 1,25-dihydroxy vitamin D by inducing monocytic stem cells to differentiate into osteoclasts and by maintaining calcium in the normal range, which results in bone mineralization by the deposition of calcium hydroxyapatite onto the bone surface, see Holick, M F, Vitamin D photobiology, metabolism, and clinical applications, In: DeGroot L, Besser H, Burger H G, eg al., eds. Endocrinology, 3^{rd} ed., 990-1013 (1995). However, elevated levels of 1α,25-dihydroxy vitamin D₃ can result in an increase of calcium concentration in the blood and in the abnormal control of calcium concentration by bone metabolism, resulting in hypercalcemia. 1α,25-dihydroxyvitamin D₃ also indirectly regulates osteoclastic activity in bone metabolism and elevated levels may be expected to increase excessive bone resorption in osteoporosis.

“Synthetic vitamin D analogues” includes non-naturally occurring compounds that act like vitamin D.

“Nonsteroidal anti-inflammatory drugs” or NSAIDs, inhibit the metabolism of arachidonic acid to proinflammatory prostaglandins via cyclooxygenase (COX)-1 and COX-2.

Nonlimiting examples of NSAIDs include: aspirin, ibuprofen, naproxen, diclofenac, etodolac, fenoporfen, flubiprofen, indomethacin, ketoprofen, ketorolac, meloxicam, nabumetone, oxaprozin, piroxicam, sulindac, tolmetin, diflunisal, meclofenamate and phenylbutazone.

A “selective cyclooxygenase-2 inhibitor,” or COX-2 inhibitor, refers to a type of nonsteroidal anti-inflammatory drug (NSAID), that inhibit the COX-2 coenzyme, which contributes to pain and inflammation in the body. Nonlimiting examples of COX-2 inhibitos include: celecoxib, etoricoxib, parecoxib, rofecoxib, valdecoxib and lumiracoxib.

An “inhibitor of interleukin-1 beta” or IL-1β refers to in inhibitors of IL-1, which is a soluble factor produced by monocytes, macrophages, and other cells which activates T-lymphocytes and potentiates their response to mitogens or antigens. Nonlimiting examples of IL-1B inhibitors include diacerein and rhein.

A “LOX/COX inhibitor” refers to an inhibitor or all three of the major enzymes involved in arachidonic acid pathway—namely, 5-LOX, COX-1 and COX-2. A nonlimiting example of a LOX/COX inhibitor is licofelone.

If formulated as a fixed dose, such combination products employ the compounds of this invention within the dosage range described below and the other pharmaceutically active agent(s) within its approved dosage range. Compounds of the instant invention may alternatively be used sequentially with known pharmaceutically acceptable agent(s) when a combination formulation is inappropriate.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents. The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985, which is incorporated by reference herein in its entirety. Metabolites of these compounds include active species produced upon introduction of compounds of this invention into the biological milieu.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The terms “treating” or “treatment” of a disease as used herein includes: preventing the disease, i.e. causing the clinical symptoms of the disease not to develop in a mammal that may be exposed to or predisposed to the disease but does not yet experience or display symptoms of the disease; inhibiting the disease, i.e., arresting or reducing the development of the disease or its clinical symptoms; or relieving the disease, i.e., causing regression of the disease or its clinical symptoms.

The term “bone resorption,” as used herein, refers to the process by which osteoclasts degrade bone.

The present invention also encompasses a pharmaceutical composition useful in the treatment of osteoporosis or other bone disorders, comprising the administration of a therapeutically effective amount of the compounds of this invention, with or without pharmaceutically acceptable carriers or diluents. Suitable compositions of this invention include aqueous solutions comprising compounds of this invention and pharmacologically acceptable carriers, e.g., saline, at a pH level, e.g., 7.4. The solutions may be introduced into a patient's bloodstream by local bolus injection.

When a compound according to this invention is administered into a human subject, the daily dosage will normally be determined by the prescribing physician with the dosage generally varying according to the age, weight, and response of the individual patient, as well as the severity of the patient's symptoms.

In one exemplary application, a suitable amount of compound is administered to a mammal undergoing treatment for a cathepsin dependent condition. Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

The compounds of the present invention can be used in combination with other agents useful for treating cathepsin-mediated conditions. The individual components of such combinations can be administered separately at different times during the course of therapy or concurrently in divided or single combination forms. The instant invention is therefore to be understood as embracing all such regimes of simultaneous or alternating treatment and the term “administering” is to be interpreted accordingly. It will be understood that the scope of combinations of the compounds of this invention with other agents useful for treating cathepsin-mediated conditions includes in principle any combination with any pharmaceutical composition useful for treating disorders related to estrogen functioning.

The scope of the invention therefore encompasses the use of the instantly claimed compounds in combination with a second agent selected from: an organic bisphosphonate; a selective estrogen receptor modulator; an androgen receptor modulator; an inhibitor of osteoclast proton ATPase; an inhibitor of HMG-CoA reductase; an integrin receptor antagonist; an osteoblast anabolic agent, such as PTH; Vitamin D; a synthetic Vitamin D analogue; a Nonsteroidal anti-inflammatory drug; a selective cyclooxygenase-2 inhibitor; an inhibitor of interleukin-1 beta; a LOX/COX inhibitor and the pharmaceutically acceptable salts and mixtures thereof.

These and other aspects of the invention will be apparent from the teachings contained herein.

### Definitions

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included in the present invention. In addition, the compounds disclosed herein may exist as tautomers and both tautomeric forms are intended to be encompassed by the scope of the invention, even though only one tautomeric structure is depicted. For example, any claim to compound A below is understood to include tautomeric structure B, and vice versa, as well as mixtures thereof.

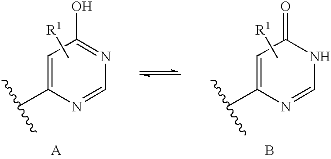

When any variable (e.g. R¹, R², R³ etc.) occurs more than one time in any constituent, its definition on each occurrence is independent at every other occurrence. Also, combinations of substituents and variables are permissible only if such combinations result in stable compounds. Lines drawn into the ring systems from substituents indicate that the indicated bond may be attached to any of the substitutable ring carbon atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to three substituents.

As used herein, “alkyl” is intended to include both branched and straight-chain saturated aliphatic hydrocarbon groups having one to ten carbon atoms unless otherwise specified. For example, C₁-C₆, as in “C₁-C₆ alkyl” is defined to include groups having 1, 2, 3, 4, 5 or 6 carbons in a linear, branched, or cyclic arrangement. For example, “C₁-C₆ alkyl” specifically includes methyl, ethyl, propyl, butyl, pentyl, hexyl, and so on.

The term “haloalkyl” means an alkyl radical as defined above, unless otherwise specified, that is substituted with one to five, preferably one to three halogen. Representative examples include, but are not limited to trifluoromethyl, dichloroethyl, and the like.

As used herein, the term “alkenyl” refers to a non-aromatic hydrocarbon radical, straight, branched or cyclic, containing from 2 to 10 carbon atoms and at least one carbon to carbon double bond. Preferably one carbon to carbon double bond is present, and up to four non-aromatic carbon-carbon double bonds may be present. Thus, “(C₂-C₆)alkenyl” means an alkenyl radical having from 2 to 6 carbon atoms. Alkenyl groups include ethenyl, propenyl, butenyl, 2-methylbutenyl and cyclohexenyl. The straight, branched or cyclic portion of the alkenyl group may contain double bonds and may be substituted if a substituted alkenyl group is indicated.

As used herein, the term “alkynyl” refers to a non-aromatic hydrocarbon radical, straight, branched or cyclic, containing from 2 to 10 carbon atoms and at least one carbon to carbon triple bond. Preferably one carbon to carbon triple bond is present, and up to four non-aromatic carbon-carbon triple bonds may be present. Thus, “(C₂-C₆)alkynyl” means an alkynyl radical having from 2 to 6 carbon atoms. Alkynyl groups include ethynyl, propynyl, butynyl, 2 methylbutynyl and cyclohexynyl. The straight, branched or cyclic portion of the alkenyl group may contain double bonds and may be substituted if a substituted alkenyl group is indicated.

The term “cycloalkyl” means a monocyclic saturated aliphatic hydrocarbon group having the specified number of carbon atoms. For example, “cycloalkyl” includes cyclopropyl, methyl-cyclopropyl, 2,2-dimethyl-cyclobutyl, 2-ethyl-cyclopentyl, cyclohexyl, and so on. “Alkoxy” represents either a cyclic or non-cyclic alkyl group of indicated number of carbon atoms attached through an oxygen bridge. “Alkoxy” therefore encompasses the definitions of alkyl and cycloalkyl above.

The term “cycloalkenyl” means a monocyclic saturated aliphatic hydrocarbon group having the specified number of carbon atoms. For example, “cycloalkenyl” includes cyclopropenyl, methyl-cyclopropenyl, 2,2-dimethyl-cyclobuentyl, 2-ethyl-cyclopentenyl, cyclohexenyl, and so on.

“Alkoxy” or “alkyloxy” represents either a cyclic or non-cyclic alkyl group of indicated number of carbon atoms attached through an oxygen bridge. “Alkoxy” therefore encompasses the definitions of alkyl and cycloalkyl above.

In certain instances, substituents may be defined with a range of carbons that includes zero, such as (C₀-C₆)alkyl-aryl. If aryl is taken to be phenyl, this definition would include phenyl itself as well as —CH₂Ph, —CH₂CH₂Ph, CH(CH₃)CH₂CH(CH₃)Ph, and so on.

The term “heterocycle” or “heterocyclyl” as used herein is intended to mean a 3- to 10-membered aromatic or nonaromatic heterocycle containing from 1 to 4 heteroatoms selected from the group consisting of O, N and S, and includes bicyclic groups. “Heterocyclyl” therefore includes the above mentioned heteroaryls, as well as dihydro and tetrathydro analogs thereof. Further examples of “heterocyclyl” include, but are not limited to the following: benzoimidazolyl, benzoimidazolonyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenafuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,41-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyidinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

As used herein, “aryl” is intended to mean any stable monocyclic or bicyclic carbon ring of up to 12 atoms in each ring, wherein at least one ring is aromatic. Examples of such aryl elements include phenyl, naphthyl, tetrahydronaphthyl, indanyl, biphenyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic, bicyclic or tricyclic ring of up to 10 atoms in each ring, wherein at least one ring is aromatic and contains from 1 to 4 heteroatoms selected from the group consisting of O, N and S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, pyranyl, pyrazinyl, pyrazolyl, pyridazinyl, pyridopyridinyl, pyridyl, pyrimidinyl, pyrrolyl, quinazolinyl, quinolyl, quinoxalinyl, tetrazolyl, tetrazolopyridyl, thiadiazolyl, thiazolyl, thienyl, triazolyl, dihydrobenzoimidazolyl, dihydrobenzofuranyl, dihydrobenzothiophenyl, dihydrobenzoxazolyl, dihydroindolyl, dihydroquinolinyl methylenedioxybenzene, benzothiazolyl, benzothienyl, quinolinyl, isoquinolinyl, oxazolyl, and tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

Whenever the term “alkyl” or “aryl” or either of their prefix roots appear in a name of a substituent (e.g., aryl C₁₋₆ alkyl) it shall be interpreted as including those limitations given above for “alkyl” and “aryl.” Designated numbers of carbon atoms (e.g., C₁₋₆) shall refer independently to the number of carbon atoms in an alkyl or cyclic alkyl moiety or to the alkyl portion of a larger substituent in which alkyl appears as its prefix root.

The terms “arylalkyl” and “alkylaryl” include an alkyl portion where alkyl is as defined above and to include an aryl portion where aryl is as defined above. Examples of arylalkyl include, but are not limited to, benzyl, fluorobenzyl, chlorobenzyl, phenylethyl, phenylpropyl, fluorophenylethyl, chlorophenylethyl, thienylmethyl, thienylethyl, and thienylpropyl. Examples of alkylaryl include, but are not limited to, toluoyl, ethylphenyl, and propylphenyl.

The term “heteroarylalkyl,” as used herein, shall refer to a system that includes a heteroaryl portion, where heteroaryl is as defined above, and contains an alkyl portion. Examples of heteroarylalkyl include, but are limited to, pyridylmethyl, pyridylethyl and imidazoylmethyl.

The term “cycloalkylalkyl,” as used herein, shall refer to a system that includes a 3- to 8-membered fully saturated cyclic ring portion and also includes an alkyl portion, wherein cycloalkyl and alkyl are as defined above.

As appreciated by those of skill in the art, “halo” or “halogen” as used herein is intended to include chloro, fluoro, bromo and iodo.

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, CH(CH₃) CH₂CH(CH₃)Ph, and so on.

The present invention also includes N-oxide derivatives and protected derivatives of compounds of Formula I. For example, when compounds of Formula I contain an oxidizable nitrogen atom, the nitrogen atom can be converted to an N-oxide by methods well known in the art. Also when compounds of Formula I contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. 1981, the disclosure of which is incorporated herein by reference in its entirety. The protected derivatives of compounds of Formula I can be prepared by methods well known in the art.

The pharmaceutically acceptable salts of the compounds of this invention include the conventional non-toxic salts of the compounds of this invention as formed inorganic or organic acids. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. The preparation of the pharmaceutically acceptable salts described above and other typical pharmaceutically acceptable salts is more fully described by Berg et al., “Pharmaceutical Salts,” J. Pharm. Sci., 1977:66:1-19, hereby incorporated by reference. The pharmaceutically acceptable salts of the compounds of this invention can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

For purposes of this specification, the following abbreviations have the indicated meanings:
AcOH=acetic acid
Boc=t-butyloxycarbonyl
Boc₂O=di-tert-butyl dicarbonate
Br₂=bromine
BuLi=butyl lithium
t-BuOH=tert-butanol
CDI=N,N′-carbonyldiimidazole
CH₂Cl₂=methylene chloride
CH₃CN=acetonitrile
Cs₂CO₃=cesium carbonate
CuI=copper iodide
DMAP=4-(dimethylamino)pyridine
DMF=N,N-dimethylformamide
DMSO=dimethylsulfoxide
DPPA=diphenylphosphoryl azide
EDCI=1-(3-dimethylaminopropyl)-3-ethylcarbodiimide hydrochloride
Et₂O=diethyl ether
Et₃N=triethylamine
EtOAc=ethyl acetate
EtOH=ethanol
Et₃SiH=triethylsilane
HATU=o-(7-azabenzotriazol-1-yl)-N,N,N′,N′-tetramethyluronium Hexafluorophosphate
HCl=hydrochloric acid
H₃PO₄=phosphoric acid
HOAc=acetic acid
HOAt=1-hydroxy-7-azabenzotriazole
K₂CO₃=potassium carbonate
KHMDS=potassium hexamethyldisilazane
KOBu^{t}=potassium tert-butoxide
LDA=lithium diisopropylamide
LiCl=lithium chloride
LiOH=lithium hydroxide
mCPBA=meta-chloroperbenzoic acid
MeMgBr=methyl magnesium bromide
MeOH=methanol
MeSO₃H=methane sulfonic acid
MgSO₄=magnesium sulfate
Ms=methanesulfonyl=mesyl
MsCl=methanesulfonyl chloride
NaBH₄=sodium borohydride
NaClO₂=sodium chlorite
NaH=sodium hydride
NaI=sodium iodide
Na₂CO₃=sodium carbonate
NaHCO₃=sodium hydrogencarbonate
NaOH=sodium hydroxide
Na₂SO₄=sodium sulfate
Na₂S₂O₃=sodium thiosulfate
NBS=N-bromosuccinimide
NH₃=ammonia
NH₄Cl=ammonium chloride
NH₄OH=ammonium hydroxide
NMR=nuclear magnetic resonance
Pd(OAc)₂=palladium acetate
Pd/C=palladium on carbon
PdCl₂=dichloropalladium(II)
PdCl₂(dppf)=[1,1′-bis(diphenylphosphino)ferrocene]dichloropalladium(II)
Pd₂(dba)₃=tris(dibenzylideneacetone)dipalladium(0)
PG=protecting group
PPh₃=triphenylphosphine
PPTS=pyridinium p-toluenesulfonate
PyBOP=benzotriazol-1-yloxytris(pyrrolidino)phosphonium-hexafluorophosphate
rt=room temperature
sat. aq.=saturated aqueous
TESCl=triethylsilyl chloride
TFA=trifluoroacetic acid
TFAA=trifluoroacetic anhydride
THF=tetrahydrofuran
TiCl₄=titanium(IV) chloride
tlc=thin layer chromatography
TMSCl=chlorotrimethylsilane
Me=methyl
Et=ethyl
n-Pr=normal propyl
i-Pr=isopropyl
n-Bu=normal butyl
i-Bu=isobutyl
s-Bu=secondary butyl
t-Bu=tertiary butyl

### Preparation of Compounds of the Invention:

The compounds of structural formula I can be prepared according to the procedures of the following Schemes and Examples, using appropriate materials and are further exemplified by the following specific examples. The compounds illustrated in the examples are not, however, to be construed as forming the only genus that is considered as the invention. The Examples further illustrate details for the preparation of the compounds of the present invention. Those skilled in the art will readily understand that known variations of the conditions and processes of the following preparative procedures can be used to prepare these compounds. All temperatures are degrees Celsius unless otherwise noted.

### Method A:

Amino alcohols of general formula 4 can be generated by following method A. The amino group of an esterified amino acid 1 can be protected with a variety of protecting groups. One such example would involve the use of benzyl chloroformate and a base such as pyridine to afford compound 2. The ester group can then be reduced using a reagent such as sodium borohydride to generate the alcohol 3. Finally, the amino protecting group can be removed to afford the amino alcohol of general formula 4 (in the case of a benzyl protecting group, palladium on carbon in conjunction with a hydrogen atmosphere can be used for the deprotection step).

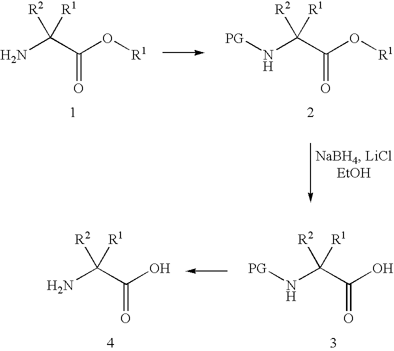

### Method B:

Amino amides of general formula 8 can be prepared following method B. When X═S, the protecting group for the amino acid 5 could be a trityl group. The amino group of 5 can be protected to afford compound 6 (for example, an FMOC group could be appended using standard amino acid chemistry). The acid 6 can be converted to the amide 7 using CD1 and NH₄OH (it is important to note that CDI should be used and not HATU to avoid racemisation of the chiral centre). Finally, the amino protecting group can be removed to afford amino amides of general formula 8 (in the case of FMOC, pyrrolidine can be used for the deprotection).

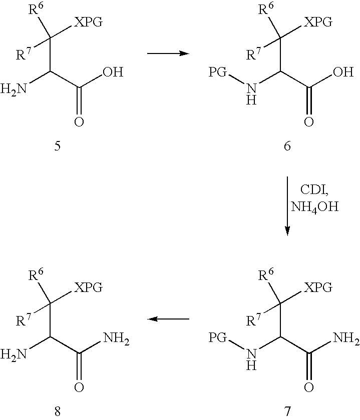

### Method C:

Acetylenes of general formula 16 can be generated following method C. Acid 9 can be reduced to alcohol 10 by first forming the mixed anhydride with isobutylchloroformate and then reducing with a reagent such as sodium borohydride. Both the alcohol and the amino group can be protected using 2-methoxypropene and PPTS to generate the oxazolidine 11. The benzyl ester can be cleaved to the acid 12 using a base such as LiOH. The acid can then be reduced to alcohol 13 using the same procedure to generate compound 10. The alcohol 13 can, in turn, be converted to the bromide 14 using standard bromination conditions. The bromide 14 can be displaced with [(trimethylsilyl)ethynyl]lithium to afford acetylene 15. Finally, the acetylene 15 can be deprotected with TBAF to generate the desired acetylene 16.

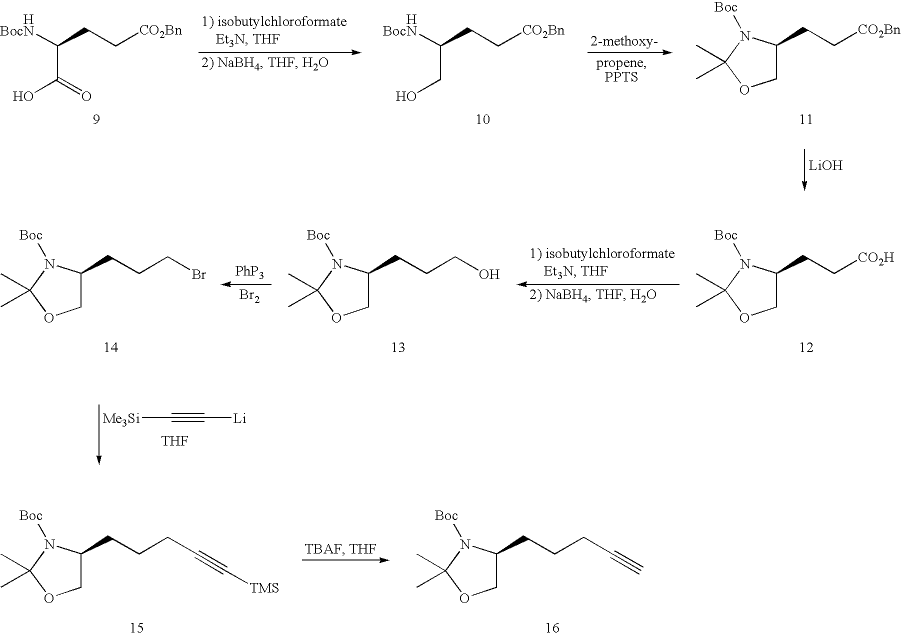

### Method D:

Compounds of structural formula I wherein X is S and Y is nitrile or amide can be prepared by method D. Amino alcohol 4 can be refluxed with ketone 17 to generate oxazolidine 18. This oxazolidine can be opened up with a lithiated acetylene 19 to generate the alcohol 20. The alcohol can then be oxidized to the acid 21 using a two step oxidation procedure (i.e. Dess Martin oxidation followed by reaction with 2-methylbut-1-ene, H₃PO₄NaClO₂). Acid 21 can then be coupled with amino amide 8 using standard peptide coupling conditions (i.e. HATU, Et₃N, DMF) to afford amide 22. The alcohol of 22 can be deprotected followed by bromination to generate the bromide 23 (in the case where the protecting group is a TES group, it can be removed under the oxidation conditions used to generate 21). The sulphur protecting group of 23 is then removed to generate a thiol (in the case where the PG is a trityl group, TFA can be used for the deprotection). With the thiol bromide precursor in hand, a base-promoted cyclization affords the macrocycle 24. Finally, the amide can be dehydrated using TFAA to afford the desired nitrile 25. Furthermore, the thiol can be oxidized with mCPBA to generate the sulfoxide 26 (m=1) or oxidized with sodium tungstate dehydrate, tetrabutylammonium hydrogen sulfate and 30% hydrogen peroxide to generate the sulfone 26 (m=2).

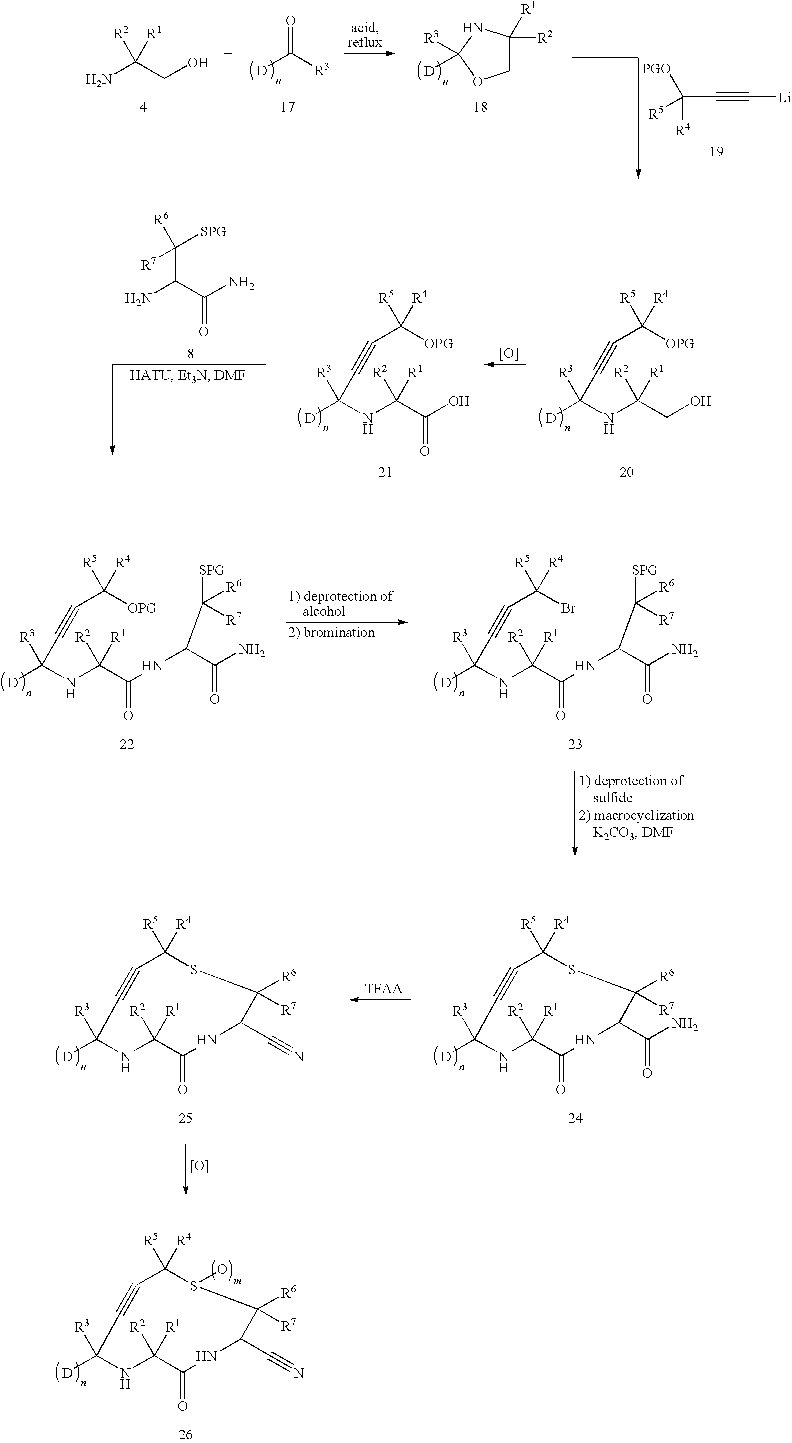

### Method E:

Compounds of structural formula I wherein X is S can be further elaborated by method E. Amide 24 can be dehydrated using TFAA to afford nitrile 25 and this, in turn, can be subjected to a Suzuki reaction under usual palladium-catalyzed conditions to afford the bicyclic compound 28 where n=2. Alternatively, the amide 24 can first undergo a Suzuki reaction to generate 27 followed by a dehydration with TFAA to afford the desired bicyclic nitrile 28.

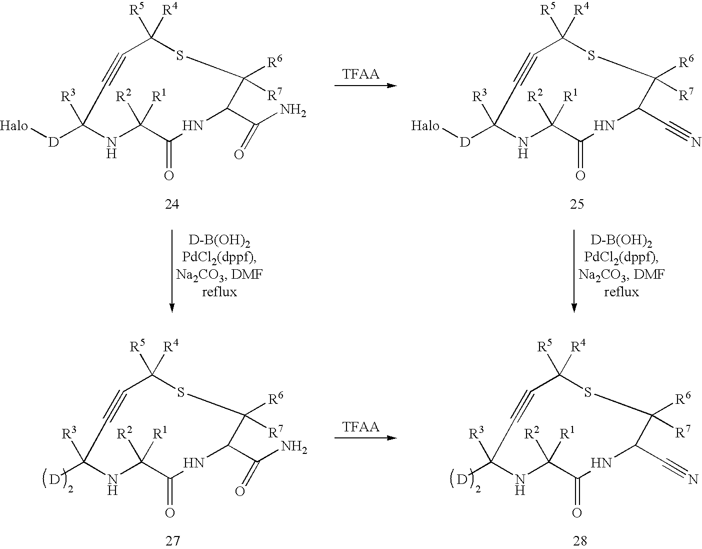

### Method F:

Compounds of structural formula I wherein X is S and Y is ester or ketone can be prepared by method F. Starting with acid 21, standard peptide coupling with an esterified amino acid 29 can generate compound 30. Deprotection of the alcohol moiety of 30 followed by standard bromination (i.e. Ph₃P.Br₂) can generate bromide 31. Deprotection of the sulphide (i.e. TFA when PG=trityl) followed by base-promoted cyclization affords the macrocycle 32 where Y=methyl ester. The methyl ester can be converted to a ketone moiety by first reducing the ester to the alcohol 33 using a reagent such as sodium borohydride. Alcohol 33 can then be oxidized to the aldehyde using an oxidant such as Dess-Martin periodinane, followed by addition of a Grignard reagent and then further oxidation of the resultant secondary alcohol to generate the desired ketone 34.

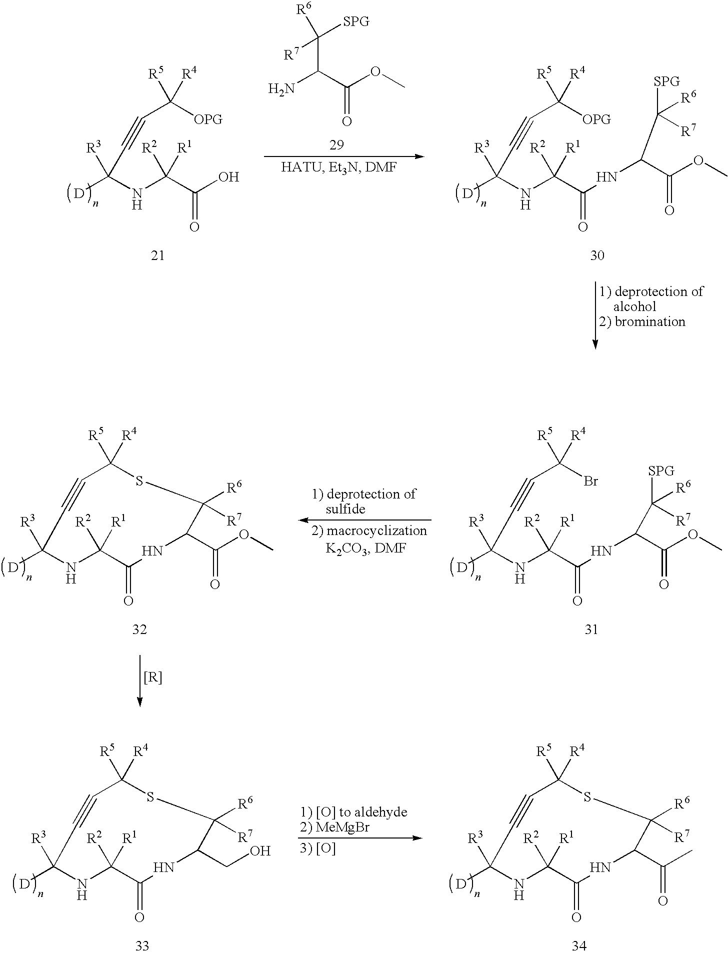

### Method G:

Compounds of structural formula I wherein X is S and Y is acid can be prepared by method G. Ester 32 can undergo a Suzuki cross-coupling reaction to generate the bicyclic compound 35 which, in turn, can be hydrolyzed to the desired acid 36 using a base such as LiOH.

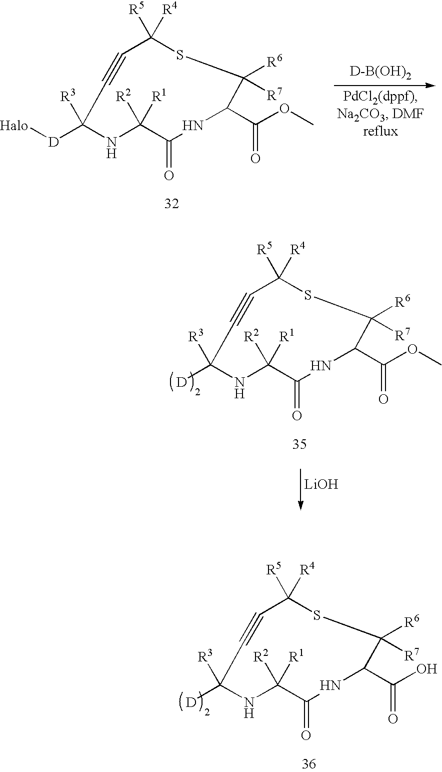

### Method H:

Compounds of structural formula I wherein X is —OC(O)— and Y is an amide or nitrile can be prepared by method H. Starting with acid 37, amino amides of general structure 38 can be coupled under standard peptide coupling conditions to generate compound 39. The ester protecting group of 39 can be removed followed by macrocyclization using 2,4,6-trichlorobenzoyl chloride to afford the desired macrocycle 40. The amide can then be dehydrated using TFAA to afford the desired nitrile macrocycle 41.

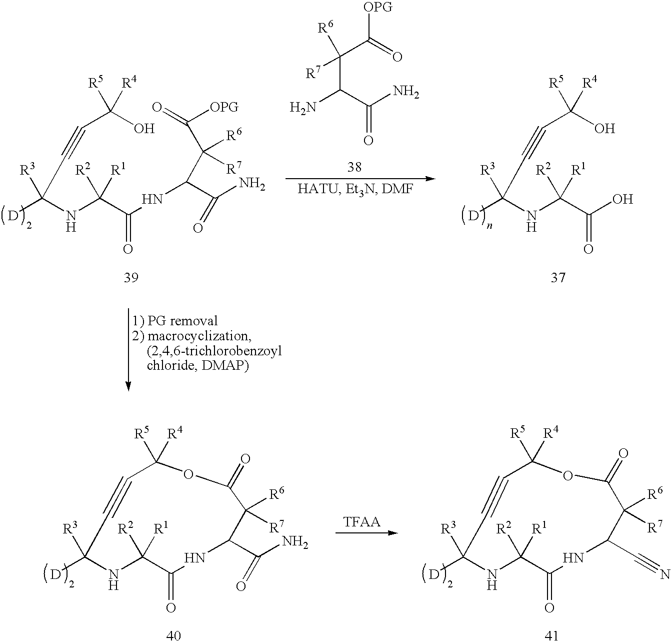

### Method I:

Compounds of structural formula I wherein X is a carbon and Y is an amide or nitrile can be prepared by method I. Acetylene 16 can be lithiated using a reagent such as butyllium and the resultant compound 42 can be used to open the oxazolidine 18 to afford 43. The alcohol of 43 can be oxidized to the acid 44 using a two step oxidation procedure (i.e. Dess Martin oxidation followed by reaction with 2-methylbut-1-ene, H₃PO₄NaClO₂). The oxazolidine protecting group of 44 can be removed by treatment with an acid such as TFA to afford amino alcohol 45. Intramolecular coupling of the amino and acid moieties with standard reagents such as HATU and Et₃N can generate the carbon macrocycle 46. The alcohol of 46 can be oxidized to the acid 47 using a two step oxidation procedure as outlined for 43→44. The acid can then be converted to the desired amide 48 using reagents such as NH₄Cl and HATU. Finally, the amide 48 can be dehydrated to the nitrile 49 using TFAA.

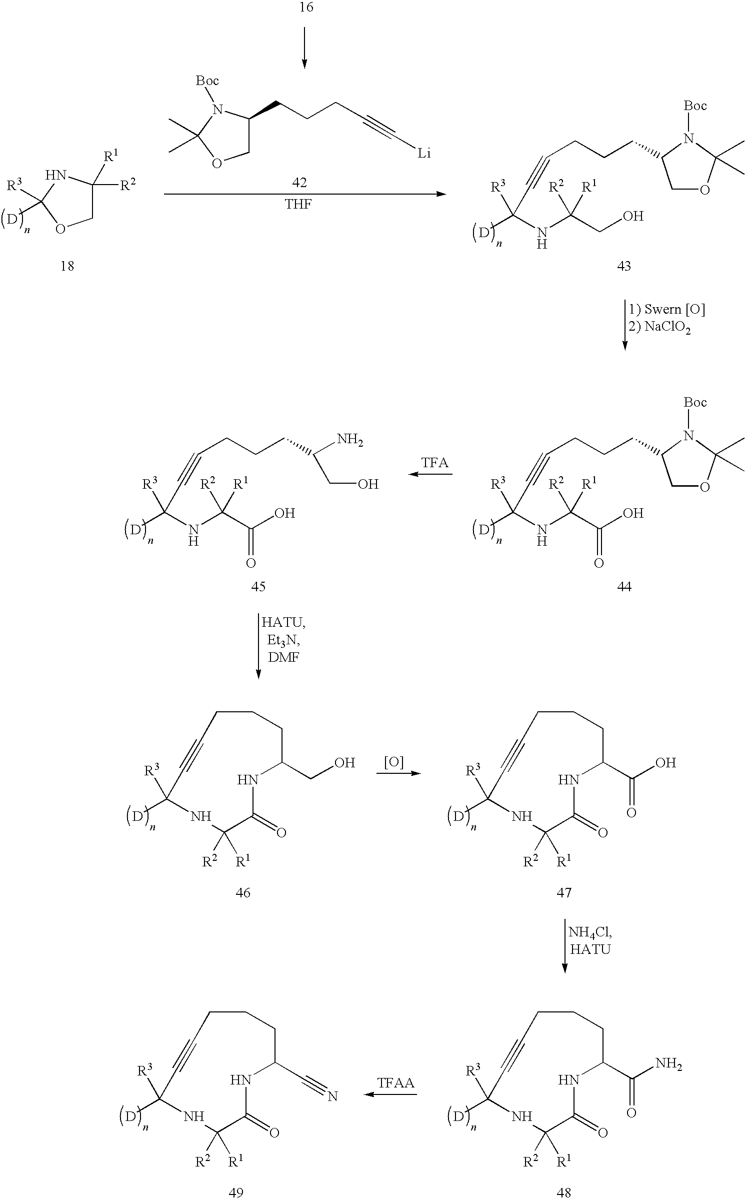

The following examples describe the synthesis of selected compounds of the current invention:

### Example 1

### Synthesis of (2S)-2-amino-4-fluoro-4-methylpentan-1-ol

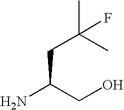

### Step 1: Ethyl N-[(benzyloxy)carbonyl]-4-fluoro-L-leucinate

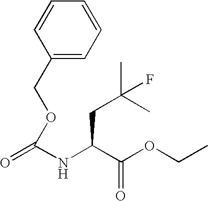

To a cold (0° C.) stirred solution of ethyl 4-fluoro-L-leucinate (Synlett, 2006, 2, 291, 19.1 g, 107.8 mmol) in acetonitrile (540 mL) was added pyridine (26 mL, 323 mmol) followed by the dropwise addition of benzyl chloroformate (16.9 mL, 118.6 mmol). The reaction was allowed to warm slowly to room temperature and subsequently stirred overnight. EtOAc was added and the mixture was washed with 10% aq. HCl (2×), brine (3×), dried (MgSO₄) and concentrated to yield the title compound as an oil.

¹H NMR (500 MHz, d₆-acetone) δ 7.38-7.28 (5H, m), 6.63 (1H, d), 5.17 (2H, s), 4.42-4.35 (1H, m), 4.12 (2H, q), 2.20 (1H, dt), 2.09-2.02 (1H, m), 1.40 (6H, dd), 1.20 (311, t).

### Step 2: Benzyl [(1S)-3-fluoro-1-(hydroxymethyl)-3-methylbutyl]carbamate

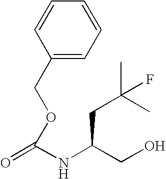

To a cold (0° C.) stirred solution of ethyl N-[(benzyloxy)carbonyl]-4-fluoro-L-leucinate (33.5 g, 107.8 mmol) in ethanol (1000 mL) was added LiCl (18.3 g, 431 mmol) followed by NaBH₄ (16.3 g, 431 mmol). The mixture was allowed to warm to room temperature and stirred for 1.5 h. By ¹H NMR, an aliquot revealed that there was mainly starting material; water (3.9 mL, 216 mmol) was added and the thick suspension was stirred overnight ('H NMR analysis indicated that the reaction was complete). The reaction was poured into 10% aq. HCl (400 mL) and the mixture was concentrated by rotary evaporation to remove the solvent. EtOAc and brine were added and the layers were separated. The aqueous layer was extracted with EtOAc (1×) and the combined organic extracts were dried (MgSO₄) and concentrated. The resulting residue was purified by column chromatography on silica gel (eluting with 30:70 EtOAc:hexanes to 70:30 EtOAc:hexanes) to afford the title compound as an oil.

¹H NMR (500 MHz, d₆-acetone) δ 7.38-7.25 (5H, m), 6.11 (1H, d), 5.10-5.02 (2H, m), 3.40-3.32 (2H, m), 3.57-3.43 (2H, m), 2.00 (1H, dt), 1.37-1.25 (1H, m), 1.36 (6H, dd).

### Step 3: (2S)-2-Amino-4-fluoro-4-methylpentan-1-ol

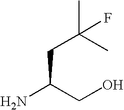

A stirred solution of benzyl [(1S)-3-fluoro-1-(hydroxymethyl)-3-methylbutyl]carbamate (25.4 g, 94.4 mmol) in EtOH (1000 mL) was degassed with nitrogen followed by the addition of 10% palladium on carbon (1.3 g, 5%). This resultant thick black suspension was evacuated and a hydrogen atmosphere was introduced via a balloon. The reaction was stirred at room temperature for 3.5 h and then filtered through celite (the celite pad was rinsed with CH₂Cl₂) and the combined filtrates were concentrated to yield the title compound as an oil.

¹H NMR (500 MHz, CD₃OD) δ 3.57-3.52 (1H, m), 3.39-3.33 (1H, m), 3.20-3.15 (1H, m), 1.82-1.65 (2H, m), 1.42 (6H, d).

### Example 2

### Synthesis of S-trityl-L-cysteinamide

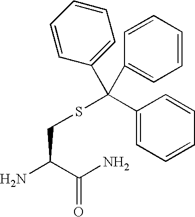

### Step 1: 9H-Fluoren-9-ylmethyl {(1R)-2-amino-2-oxo-1-[(tritylthio)methyl]-ethyl}carbamate

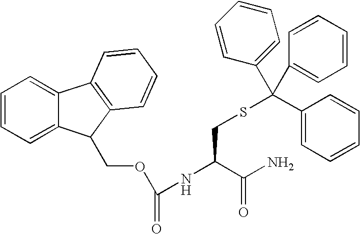

To a stirred suspension of N-[9H-fluoren-9-ylmethoxy)carbonyl]-5-trityl-L-cysteine (2.64 g, 4.5 mmol) in CH₂Cl₂ (44 mL) was added CDI (0.95 g, 5.9 mmol). After the resultant solution stopped bubbling, NH₄OH (1.2 mL, 18 mmol) was added and the reaction was stirred at room temperature for 3 h. EtOAc and water were added and the layers were separated. The organic layer was washed with 10% aq. HCl (1×), sat. aq. NaHCO₃ (1×), brine (1×), dried (MgSO₄) and concentrated. The residue was triturated with Et₂O/hexane and filtered. The filtrate was concentrated and re-triturated with hexane. The combined precipitates were analyzed by rotation ([α]=+11 (MeOH, c=0.5)) and chiral HPLC (AD column, 1:1 iPrOH/hexane, one enantiomer at 6.62 min) which indicated that the title compound was obtained in high chiral purity. It is important to note that if this reaction is conducted with HATU instead of CDI, complete racemization of the chiral center occurs.

¹H NMR (500 MHz, d₆-acetone) δ 7.89 (2H, d), 7.70 (2H, d), 7.60 (1H, d), 7.40 (1H, br t), 7.35-7.22 (18H, m), 7.10 (1H, s), 4.30-4.22 (3H, m), 4.02-3.97 (1H, m), 2.40-2.33 (2H, m).

### Step 2: S-Trityl-L-cysteinamide

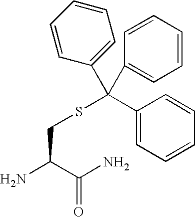

To a stirred solution of 9H-fluoren-9-ylmethyl {(1R)-2-amino-2-oxo-1-[(tritylthio)methyl]-ethyl}carbamate (380 mg, 0.65 mmol) in DMF (3 mL) was added pyrrolidine (11 μL, 0.13 mmol). The reaction was stirred at room temperature overnight. By TLC and mass spectrometry, the reaction was deemed complete. This DMF solution of the title compound was used as such in the next reaction (Step 5, Example 3).

### Example 3

### Synthesis of (3R,6S,8R)-8-(4-bromophenyl)-6-(2-fluoro-2-methylpropyl)-5-oxo-8-(trifluoromethyl)-1-thia-4,7-diazacycloundec-9-yne-3-carbonitrile

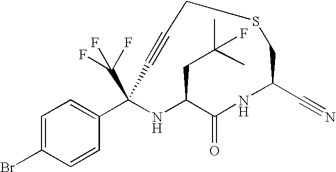

### Step 1: (2R,4S)-2-(4-Bromophenyl)-4-(2-fluoro-2-methylpropyl)-2-(trifluoromethyl)-1,3-oxazolidine

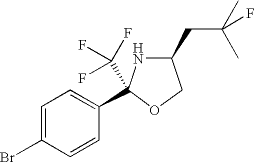

A stirred solution of 1-(4-bromophenyl)-2,2,2-trifluoroethanone (17.7 g, 70 mmol), (2S)-2-amino-4-fluoro-4-methylpentan-1-ol from Step 3, Example 1 (9.45 g, 70 mmol) and PPTS (880 mg, 3.5 mmol) in toluene (300 mL) was heated to reflux (oil bath temperature at 130° C.) with a dean start apparatus for 2 days (following procedure in Tetrahedron Letters, 1998, 39, 1199). The reaction was cooled and concentrated and the two diastereomers were separated by column chromatography on silica gel eluting with 3% EtOAc/hexanes→5% EtOAc/hexanes→10% EtOAc/hexanes. The more polar diastereomer was determined to be the title compound (by comparison with above literature reference) and was formed in a 1.9:1 ratio.

### Step 2: Triethyl(prop-2-yn-1-yloxy)silane

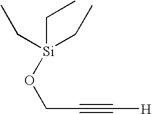

To a stirred solution of propargyl alcohol (14.8 g, 264 mmol) and imidazole (21.6 g, 317 mmol) in DMF (130 mL) was added TESCl (49 mL, 290 mmol). The reaction was stirred at room temperature for 1 h. The mixture was then partitioned between 1% aqueous Na₂CO₃ (400 mL) and hexanes (400 mL). The organic layer was separated and washed with 1% aqueous Na₂CO₃ (1×), brine (1×), dried (MgSO₄) and concentrated to afford the title compound.

### Step 3: (2S)-2-{[(1R)-1-(4-Bromophenyl)-4-[(triethylsilyl)oxy]-1-(trifluoromethyl)but-2-yn-1-yl]amino}-4-fluoro-4-methylpentan-1-ol

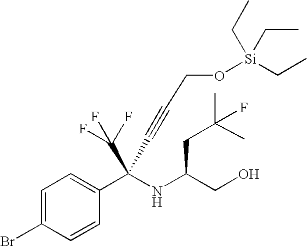

To a stirred, cold (−20° C.) solution of triethyl(prop-2-yn-1-yloxy)silane from Step 2, Example 3 (2.5 g, 14.7 mmol) in THF (17 mL) was added nBuLi (2.5 M in hexanes, 5.9 mL, 14.7 mmol). The reaction was stirred at −20° C. for 30 min.

To a stirred, cold (−78° C.) solution of (2R,4S)-2-(4-bromophenyl)-4-(2-fluoro-2-methylpropyl)-2-(trifluoromethyl)-1,3-oxazolidine from Step 1, Example 3 (1.05 g, 2.84 mmol) in THF (28 mL) was added the above formed solution of {3-[(triethylsilyl)oxy]prop-1-yn-1-yl}lithium in THF. The mixture was then stirred at −78° C. overnight and then warmed to 0° C. for 2.5 h. Saturated aq. NH₄Cl was added and the aqueous layer was extracted with EtOAc (3×). The combined organics were washed with brine (1×), dried (MgSO₄) and concentrated. The residue was purified by column chromatography on silica gel eluting with 10% EtOAc/hexanes→20% EtOAc/hexanes to afford the title compound.

### Step 4: N-[(1R)-1-(4-Bromophenyl)-4-hydroxy-1-(trifluoromethyl)but-2-yn-1-yl]-4-fluoro-L-leucine

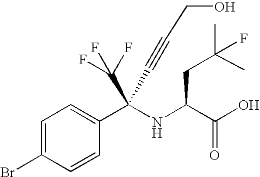

To a stirred, cold (−20° C.) solution of (2S)-2-{[(1R)-1-(4-bromophenyl)-4-[(triethylsil)oxy]-1-(trifluoromethyl)but-2-yn-1-yl]amino}-4-fluoro-4-methylpentan-1-ol from Step 3, Example 3 (722 mg, 1.34 mmol) in CH₂Cl₂ (13 mL) was added Dess Martin periodinane (850 mg, 2.00 mmol). The reaction was then warmed to room temperature and stirred for 1 h. Water (˜1 mL) was added and the mixture was stirred at room temperature for 30 min. The mixture was then quickly subjected to a column chromatography on silica gel eluting with 2% EtOAc/hexanes to afford the aldehyde (¹H NMR (500 MHz, d₆-acetone) δ 9.50 (1H, s), 7.80 (2H, d), 7.63 (2H, d), 4.55 (2H, s), 4.87-4.83 (1H, m), 3.41 (1H, br d), 2.22-2.05 (2H, m), 1.47 (6H, dd), 0.98 (9H, t), 0.66 (6H, q). This crude aldehyde was dissolved in t-BuOH (6 mL) and water (6 mL) and cooled to 0° C. To this solution was added 2-methylbut-1-ene (0.78 mL, 7.35 mmol) followed by H₃PO₄ (1.2 M in water, 3.3 mL, 4.01 mmol) and NaClO₂ (1 M in water, 3.3 mL, 3.34 mmol). The reaction was warmed to room temperature and stirred for 1.5 h. Water and EtOAc were added and the aqueous layer was extracted with EtOAc (3×). The combined organics were washed with brine (1×), dried (MgSO₄) and concentrated to afford the title compound.

¹H NMR (500 MHz, d₆-acetone) δ 7.77 (2H, d), 7.60 (2H, d), 4.38 (2H, s), 4.05-3.96 (1H, s), 3.85 (1H, br t), 2.10-2.05 (2H, m), 1.47 (6H, dd).

### Step 5: N-[(1R)-1-(4-Bromophenyl)-4-hydroxy-1-(trifluoromethyl)but-2-yn-1-yl]-4-fluoro-L-leucyl-S-trityl-L-cysteinamide

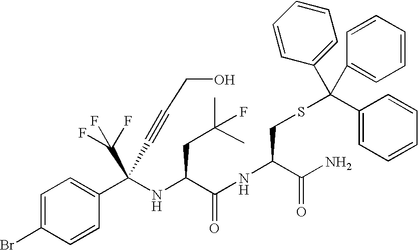

To a solution of S-trityl-L-cysteinamide from Step 2, Example 2 (235 mg, 0.65 mmol) in DMF (5 mL) was added HATU (209 mg, 0.55 mol), HOAt (68 mg, 0.5 mmol) and the N-[(1R)-1-(4-bromophenyl)-4-hydroxy-1-(trifluoromethyl)but-2-yn-1-yl]-4-fluoro-L-leucine from Step 4, Example 3 (220 mg, 0.5 mmol). The mixture was cooled to 0° C. and Et₃N (0.35 mL, 2.5 mmol) was added. The reaction was stirred at room temperature overnight. Sat. aq. NaHCO₃, EtOAc and water were added and the aqueous layer was extracted with EtOAc (3×). The combined organics were washed with brine (1×), dried (MgSO₄) and concentrated. The residue thus obtained was purified by column chromatography on silica gel eluting with 40% EtOAc/hexanes→60% EtOAc/hexanes→100% EtOAc to afford the title compound.

¹H NMR (500 MHz, d₆-acetone) δ 7.8 (2H, d), 7.65-7.20 (17H, m), 6.6 and 6.8 (2H, 2 bs (NH₂)), 4.5 (1H, m), 4.35 (2H, m), 4.25 (1H, m), 3.7 (1H, m), 3.3 (1H, m), 2.55-2.45 (2H, m), 2.25-2.15 (2H, m), 1.50-1.35 (6H, m).

### Step 6: N-[(1R)-4-Bromo-1-(4-bromophenyl)-1-(trifluoromethyl)but-2-yn-1-yl]-4-fluoro-L-leucyl-S-trityl-L-cysteinamide

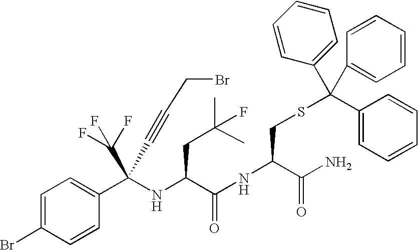

To a stirred, cold (0° C.) solution of triphenylphosphine (161 mg, 0.61 mmol) in THF (3 mL) was added Br₂ (32 μL, 0.61 mmol). To this yellow suspension was added N-[(1R)-1-(4-Bromophenyl)-4-hydroxy-1-(trifluoromethyl)but-2-yn-1-yl]-4-fluoro-L-leucyl-5-trityl-L-cysteinamide from Step 5, Example 3 (241 mg, 0.31 mmol) as a solution in THF (1 mL). The resultant yellow suspension was stirred at 0° C. for 30 min and then warmed to room temperature and stirred for 1 h. The mixture was then quenched with freshly prepared sat. aq. Na₂S₂O₃ and Et₂O was added. The aqueous layer was extracted with Et₂O (3×) and the combined organic layers were washed with sat. aq. Na₂S₂O₃ (1×), brine (1×), dried (MgSO₄) and concentrated. The residue thus obtained was purified by column chromatography eluting with 40% EtOAc/hexanes→60% EtOAc/hexanes to afford the title compound.

¹H NMR (500 MHz, d₆-acetone) δ 7.77 (2H, d), 7.60 (111, d), 7.50 (2H, d), 7.40-7.22 (15H, m), 6.80 (1H, br s), 6.64 (1H, br s), 4.30-4.23 (3H, m), 3.61 (1H, br q), 3.30 (1H, t), 2.59-2.45 (2H, m), 2.23-2.12 (2H, m), 1.40 (6H, t).

### Step 7: N-[(1R)-4-Bromo-1-(4-bromophenyl)-1-(trifluoromethyl)but-2-yn-1-yl]-4-fluoro-L-leucyl-L-cysteinamide

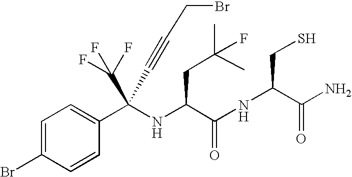

To a solution of N-[(1R)-4-bromo-1-(4-bromophenyl)-1-(trifluoromethyl)but-2-yn-1-yl]-4-fluoro-L-leucyl-5-trityl-L-cysteinamide from Step 6, Example 3 (95 mg, 0.11 mmol) and Et₃SiH (36 μL, 0.22 mmol) in CH₂Cl₂ (0.5 mL) was added TFA (0.5 mL). The reaction was stirred at room temperature for 5 min followed by removal of the solvent (using heptane to azeotrope off the TFA). The residue thus obtained was purified by column chromatography eluting with 40% EtOAc/hexanes→60% EtOAc/hexanes to afford the title compound.

¹H NMR (500 MHz, d₆-acetone) δ 7.77 (2H, d), 7.65 (1H, d), 7.58 (2H, d), 6.95 (1H, br s), 6.68 (1H, br s), 4.38-4.35 (3H, m), 3.71 (1H, br q), 3.34 (1H, br t), 2.78-2.68 (2H, m), 2.18 (2H, dd), 1.60 (1H, t), 1.45 (6H, dd).

### Step 8: (3R,6S,8R)-8-(4-Bromophenyl)-6-(2-fluoro-2-methylpropyl)-5-oxo-8-(trifluoromethyl)-1-thia-4,7-diazacycloundec-9-yne-3-carboxamide

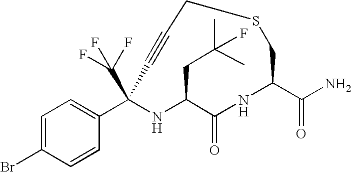

To a stirred solution of K₂CO₃ (63 mg, 0.45 mmol) in DMF (10 mL) was added slowly over 4 h, via syringe pump, a solution of N-[(1R)-4-bromo-1-(4-bromophenyl)-1-(trifluoromethyl)but-2-yn-1-yl]-4-fluoro-L-leucyl-L-cysteinamide from Step 8, Example 3 (55 mg, 0.09 mmol) in DMF (10 mL, 0.01 M concentration). The reaction was stirred at room temperature overnight. Water and EtOAc were added and the aqueous layer was extracted with EtOAc (4×). The combined organic extracts were washed with brine (1×), dried (MgSO₄) and concentrated (the residual DMF was removed by co-evaporation with heptane) to afford the title macrocylic compound in very good purity.

¹H NMR (500 MHz, d₆-acetone) δ 7.89 (1H, br s), 7.79 (2H, d), 7.65 d), 7.02 (1H, br s), 6.62 (1H, br s), 4.75 (1H, br d), 4.11 (1H, dt), 3.64 (1H, d), 3.45 (1H, d), 3.32-3.12 (1H, dd), 3.12 (1H, dd), 2.90 (1H, d), 2.10-2.05 (1H, m), 1.95-1.85 (1H, m), 1.55 (6H, dd).

MS (+ESI): 524.2, 526.2 [M+1]+

### Step 9: (3R,6S,8R)-8-(4-Bromophenyl)-6-(2-fluoro-2-methylpropyl)-5-oxo-8-(trifluoromethyl)-1-thia-4,7-diazacycloundec-9-yne-3-carbonitrile

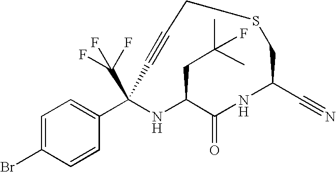

To a stirred, cold (0° C.) solution of (3R,6S,8R)-8-(4-bromophenyl)-6-(2-fluoro-2-methylpropyl)-5-oxo-8-(trifluoromethyl)-1-thia-4,7-diazacycloundec-9-yne-3-carboxamide from Step 8, Example 3 (49 mg, 0.09 mmol) and pyridine (38 μL, 0.47 mmol) in 1,4-dioxane (1 mL) was added freshly distilled TFAA (33 μL, 0.23 mmol)). The reaction was warmed to room temperature and stirred for 30 min. EtOAc and sat. aq. NaHCO₃ were added and the aqueous layer was extracted with EtOAc (1×). The combined organic extracts were washed with brine (1×), dried (MgSO₄) and concentrated. The residue thus obtained was purified by column chromatography eluting with 10% EtOAc/hexanes→30% EtOAc/hexanes to afford the title compound.

¹H NMR (500 MHz, d₆-acetone) δ 8.60 (1H, d, J=8.64 Hz), 7.78 (2H, d, J=8.37 Hz), 7.65 (2H, d, J=8.53 Hz), 5.35-5.29 (1H, m), 4.10-4.00 (1H, m), 3.81 (1H, d, J=17.78 Hz), 3.55 (1H, d, J=17.78 Hz), 3.38-3.25 (2H, m), 2.91-2.82 (1H, m), 2.00-1.92 (2H, m), 1.53 (6H, dd, J=21.68, 16.48 Hz).

MS (+ESI): 506, 508 [M+1]+

### Example 4

### Synthesis of (3R,6S,8R)-8-(4-bromophenyl)-6-(2-fluoro-2-methylpropyl)-5-oxo-8-(trifluoromethyl)-1-thia-4,7-diazacycloundec-9-yne-3-carbonitrile 1-oxide

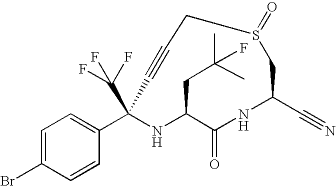

To a stirred, cold (0° C.) solution of (3R,6S,8R)-8-(4-bromophenyl)-6-(2-fluoro-2-methylpropyl)-5-oxo-8-(trifluoromethyl)-1-thia-4,7-diazacycloundec-9-yne-3-carbonitrile from Step 9, Example 3 (10.5 mg, 0.02 mmol) in CH₂Cl₂ (0.5 mL) was added mCPBA (76% pure, 5 mg, 0.023 mmol). The mixture was stirred at 0° C. for 2 h. A few drops of Me₂S were added followed by Ca(OH)₂ (200 mg) and CH₂Cl₂ (2 mL). The mixture was stirred at room temperature for 30 min and then filtered through celite. The filtrate was concentrated and purified by column chromatography on silica gel eluting with 50% EtOAc/hexanes. The fractions contained the least polar spot were concentrated to afford the title compound.

¹H NMR (500 MHz, d₆-acetone) δ 8.24 (1H, d, J=8.90 Hz), 7.79 (2H, d, J=8.30 Hz), 7.64 (2H, d, J=8.32 Hz), 5.70 (1H, s), 4.40-4.35 (1H, m), 4.03-3.93 (2H, m), 3.85-3.77 (1H, m), 3.63 (1H, d, J=3.57 Hz), 2.95 (1H, d, J=11.70 Hz), 1.97-1.91 (2H, m), 1.50 (6H, dd, J=21.71, 15.79 Hz).

MS (+ESI): 522, 524 [M+1]+

### Example 5

### Synthesis of (3R,6S,8R)-8-(4-bromophenyl)-6-(2-fluoro-2-methylpropyl)-5-oxo-8-(trifluoromethyl)-1-thia-4,7-diazacycloundec-9-yne-3-carbonitrile 1,1-dioxide

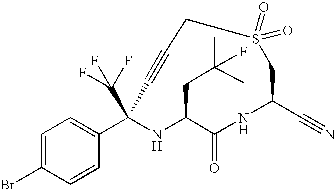

To a stirred, cold (0° C.) suspension of (3R,6S,8R)-8-(4-bromophenyl)-6-(2-fluoro-2-methylpropyl)-5-oxo-8-(trifluoromethyl)-1-thia-4,7-diazacycloundec-9-yne-3-carbonitrile from Step 9, Example 3 (107 mg, 0.211 mmol), sodium tungstate dehydrate (3.46 mg, 0.0105 mmol) and tetrabutylammonium hydrogen sulfate (3.4 mg, 0.0105 mmol) in ethyl acetate (5 mL) was added dropwise 30% hydrogen peroxide (0.0538 mL, 0.527 mmol) and the mixture was stirred at room temperature for 6 hrs. The mixture was then diluted with ethyl acetate and washed with aqueous sodium thiosulfate and brine. The organic layer was dried with magnesium sulfate and the solvent was removed in vacuo. The residue was purified on silica gel using ethyl acetate and hexanes (1:2) to afford the title compound (95 mg).

¹H NMR (500 MHz, d₆-acetone) δ 8.7-8.6 (1H, NH), 7.8-7.6 (4H, m), 5.6-5.56 (1H, bm), 4.6-4.5 (2H, m), 4.1-4.0 (2H, m), 3.1-3.0 (1H, m), 2.0 (1H, m), 1.60-1.45 (6H, m).

MS (+ESI): 537.6, 539.8 [M+1]⁺

### Example 6

### Synthesis of (3R,6S,8R)-6-(2-fluoro-2-methylpropyl)-8-[4′-(methylsulfonyl)biphenyl-4-yl]-5-oxo-8-(trifluoromethyl)-1-thia-4,7-diazacycloundec-9-yne-3-carbonitrile

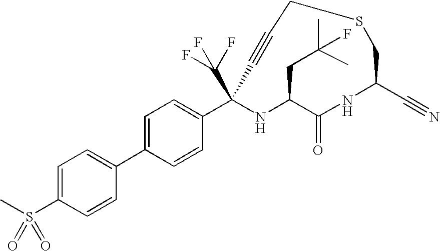

A vial containing (3R,6S,8R)-8-(4-bromophenyl)-6-(2-fluoro-2-methylpropyl)-5-oxo-8-(trifluoromethyl)-1-thia-4,7-diazacycloundec-9-yne-3-carbonitrile from Step 9, Example 3 (9.7 mg, 0.02 mmol), [4-(methylsulfonyl)phenyl]boronic acid (5 mg, 0.025 mmol), Pd(OAc)₂ (0.4 mg, 0.002 mmol), and tri(o-tolyl)phosphine (1.4 mg, 0.004 mmol) was flushed with nitrogen followed by the addition of THF (0.3 mL) and Na₂CO₃ (2M aq., 14 μL, 0.028 mmol). The mixture was heated to 67° C. and the reaction was stirred for 3 h. EtOAc and water were added and the aqueous layer was extracted with EtOAc (3×). The combined organics were washed with brine (1×), dried (MgSO₄) and concentrated. The residue thus obtained was purified by column chromatography on silica gel eluting with 30% EtOAc/hexanes→60% EtOAc/hexanes to afford the title compound.

¹H NMR δ (ppm)(Acetone): 8.58 (1H, d, J=8.67 Hz), 8.04 (2H, d, J=8.18 Hz), 7.95 (4H, dd, J=8.17, 5.73 Hz), 7.81 (2H, d, J=8.24 Hz), 5.32-5.29 (1H, m), 4.07-4.03 (1H, m), 3.82 (1H, d, J=17.81 Hz), 3.56 (1H, d, J=17.81 Hz), 3.36-3.26 (2H, m), 3.17 (3H, s), 2.93 (1H, d, J=11.8 Hz), 1.99-1.93 (2H, m), 1.53 (6H, dd, J=21.68, 16.50 Hz).

MS (+ESI): 581.8 [M+1]⁺

### Example 7

### Synthesis of (3R,6S,8R)-3-acetyl-8-(4-bromophenyl)-6-(2-methylprop-2-en-1-yl)-8-(trifluoromethyl)-1-thia-4,7-diazacycloundec-9-yn-5-one

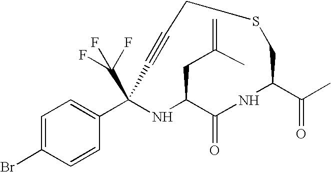

### Step 1: Methyl S-trityl-L-cysteinate

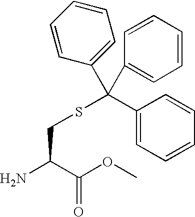

To a stirred, colourless solution of N-[(9H-fluoren-9-ylmethoxy)carbonyl]-S-trityl-L-cysteine (3.86 g, 6.6 mol) in THF was added a solution of diazomethane in Et₂O until a yellow colour persisted. The THF was then removed by rotary evaporation and the crude methyl ester (methyl N-[(9H-fluoren-9-ylmethoxy)carbonyl]-S-trityl-L-cysteinate, 3.55 g, 5.9 mol) was re-dissolved in THF (60 mL) followed by the addition of pyrrolidine (6 mL, 0.07 mol). The mixture was stirred at room temperature for 1 h followed by the removal of the solvent by rotary evaporation. The residue was co-evaporated with toluene (3×) and the crude title compound was dissolved in DMF (5.9 mL) and used as such in the next reaction.

### Step 2: Methyl N-[(1R)-1-(4-bromophenyl)-4-hydroxy-1-(trifluoromethyl)but-2-yn-1-yl]-4-fluoro-L-leucyl-5-trityl-L-cysteinate

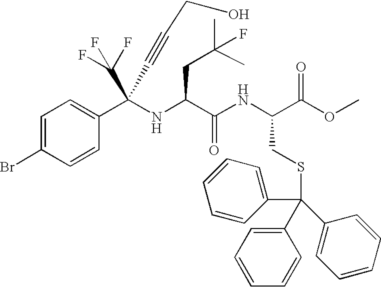

To a solution of methyl S-trityl-L-cysteinate from Step 1, Example 7 (1.1 g, 2.8 mmol) in DMF (21 mL) was added HATU (894 mg, 2.35 mol), HOAt (291 mg, 2.14 mmol) and the N-[(1R)-1-(4-bromophenyl)-4-hydroxy-1-(trifluoromethyl)but-2-yn-1-yl]-4-fluoro-L-leucine from Step 4, Example 3 (941 mg, 2.14 mmol). The mixture was cooled to 0° C. and Et₃N (1.5 mL, 10.7 mmol) was added. The reaction was stirred at room temperature overnight. Sat. aq. NaHCO₃, EtOAc and water were added and the aqueous layer was extracted with EtOAc (3×). The combined organics were washed with brine (1×), dried (MgSO₄) and concentrated. The residue thus obtained was purified by column chromatography on silica gel eluting with 20% EtOAc/hexanes→40% EtOAc/hexanes→100% EtOAc to afford the title compound.

¹H NMR δ (ppm)(Acetone): 7.75 (2H, d), 7.62 (1H, br d), 7.49 (2H, d), 7.40-7.24 (15H, m), 4.42 (1H, t), 4.35 (2H, d), 4.22 (1H, q), 3.71 (1H, q), 3.51 (3H, s), 3.24 (1H, t), 2.60-2.51 (2H, m), 2.17-2.06 (2H, m), 1.42 (6H, dd).

MS (−ESI): 797, 799 [M−1]⁺

### Step 3: Methyl N-[(1R)-4-bromo-1-(4-bromophenyl)-1-(trifluoromethyl)but-2-yn-1-yl]-4-fluoro-L-leucyl-5-trityl-L-cysteinate

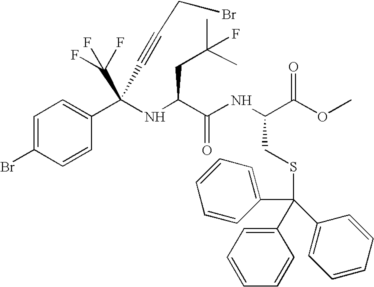

To a stirred, cold (0° C.) solution of triphenylphosphine (602 mg, 2.3 mmol) in THF (8 mL) was added Br₂ (120 μL, 2.3 mmol). To this yellow suspension was added methyl N-[(1R)-1-(4-bromophenyl)-4-hydroxy-1-(trifluoromethyl)but-2-yn-1-yl]-4-fluoro-L-leucyl-5-trityl-L-cysteinate from Step 2, Example 7 (918 mg, 1.15 mmol) as a solution in THF (3 mL). The resultant yellow suspension was warmed to room temperature and stirred for 1 h. The mixture was then quenched with freshly prepared sat. aq. Na₂S₂O₃ and Et₂O was added. The aqueous layer was extracted with Et₂O (4×) and the combined organic layers were washed with sat. aq. Na₂S₂O₃ (1×), brine (1×), dried (MgSO₄) and concentrated. The residue thus obtained was purified by column chromatography eluting with 20% EtOAc/hexanes→30% EtOAc/hexanes to afford the title compound.

MS (+ESI): 885.4, 887.4 [M+1+Na]⁺

### Step 4: Methyl N-[(1R)-4-bromo-1-(4-bromophenyl)-1-(trifluoromethyl)but-2-yn-1-yl]-4-fluoro-L-leucyl-L-cysteinate

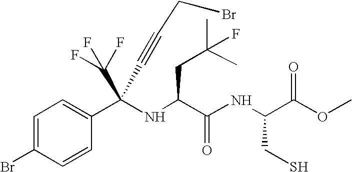

To a solution of methyl N-[(1R)-4-bromo-1-(4-bromophenyl)-1-(trifluoromethyl)but-2-yn-1-yl]-4-fluoro-L-leucyl-5-trityl-L-cysteinate from Step 3, Example 7 (742 mg, 0.86 mmol) and Et₃SiH (0.28 mL, 1.72 mmol) in CH₂Cl₂ (7 mL) was added TFA (5 mL). The reaction was stirred at room temperature for 5 min followed by removal of the solvent (using heptane to azeotrope off the TFA). The residue thus obtained was purified by column chromatography eluting with 30% EtOAc/hexanes to afford the title compound.

### Step 5: Methyl (3R,6S,8R)-8-(4-bromophenyl)-6-(2-fluoro-2-methylpropyl)-5-oxo-8-(trifluoromethyl)-1-thia-4,7-diazacycloundec-9-yne-3-carboxylate

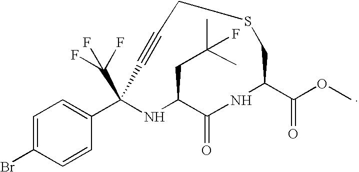

To a stirred solution of K₂CO₃ (538 mg, 3.89 mmol) in DMF (85 mL) was added slowly over 4 h, via syringe pump, a solution of methyl N-[(1R)-4-bromo-1-(4-bromophenyl)-1-(trifluoromethyl)but-2-yn-1-yl]-4-fluoro-L-leucyl-L-cysteinate from Step 4, Example 7 (486 mg, 0.78 mmol) in DMF (80 mL, 0.01 M concentration). The reaction was stirred at room temperature overnight. Water and EtOAc were added and the aqueous layer was extracted with EtOAc (3×). The combined organic extracts were washed with brine (2×), dried (MgSO₄) and concentrated (the residual DMF was removed by co-evaporation with heptane). The residue thus obtained was purified by preparatory chiral HPLC (using AD column and eluting with 40% iPrOH/hexanes) to afford the title compound.

¹H NMR δ (ppm)(Acetone): 8.03 (1H, br d), 7.76 (2H, d, J=8.30 Hz), 7.63 (2H, d, J=8.41 Hz), 4.88-4.83 (1H, m), 4.11-4.05 (1H, m), 3.71 (3H, s), 3.67 (1H, d), 3.46 (1H, d), 3.26 (1H, d, J=5.00 Hz), 3.17 (1H, d, J=3.80 Hz), 2.87 (1H, d), 1.93-1.87 (2H, m), 1.55-1.48 (6H, m).

MS (+ESI): 538.8, 540.8 [M+1]⁺

### Step 6: (3R,6S,8R)-8-(4-Bromophenyl)-6-(2-fluoro-2-methylpropyl)-5-oxo-8-(trifluoromethyl)-1-thia-4,7-diazacycloundec-9-yne-3-carboxylic acid

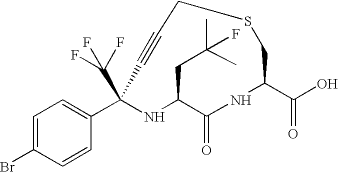

To a solution of methyl (3R,6S,8R)-8-(4-bromophenyl)-6-(2-fluoro-2-methylpropyl)-5-oxo-8-(trifluoromethyl)-1-thia-4,7-diazacycloundec-9-yne-3-carboxylate from Step 5, Example 7 (65 mg, 0.12 mmol) in DME (0.9 mL) and MeOH (0.3 mL) was added a 2M aqueous solution of LiOH (0.3 mL, 0.6 mmol). The resultant suspension was stirred at room temperature overnight. The mixture was acidified with 10% aq. HCl and the aqueous layer was extracted with EtOAc (5×). The combined organics were washed with brine (1×), dried (MgSO₄) and concentrated. The residue thus obtained was purified by column chromatography eluting with 60% EtOAc/hexanes→59% EtOAc/hexanes/1% acetic acid to afford the title compound.

¹H NMR δ (ppm)(Acetone): 7.93 (1H, d, J=7.79 Hz), 7.77 (2H, d, J=8.34 Hz), 7.63 (2H, d, J—8.41 Hz), 4.82 (1H, dd, J=8.00, 4.14 Hz), 4.10 (1H, s), 3.72-3.67 (1H, m), 3.47-3.42 (1H, m), 3.34 (1H, dd, J=14.65, 4.52 Hz), 3.18 (1H, dd, J=14.64, 3.91 Hz), 2.87 (1H, d, J=12.5 Hz), 1.96-1.87 (2H, m), 1.55-1.48 (6H, m).

MS (+ESI): 524.7, 526.7 [M+1]⁺

### Step 7: (3R,6S,8R)-8-(4-Bromophenyl)-6-(2-fluoro-2-methylpropyl)-N-methoxy-N-methyl-5-oxo-8-(trifluoromethyl)-1-thia-4,7-diazacycloundec-9-yne-3-carboxamide

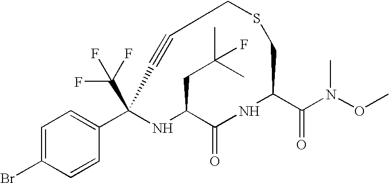

To a stirred, cold (0° C.) solution of (3R,6S,8R)-8-(4-bromophenyl)-6-(2-fluoro-2-methylpropyl)-5-oxo-8-(trifluoromethyl)-1-thia-4,7-diazacycloundec-9-yne-3-carboxylic acid from Step 6, Example 7 (24 mg, 0.05 mmol), HATU (38 mg, 0.1 mmol) and N,O-dimethylhydroxylamine (11.6 mg, 0.012 mmol) in DMF (0.5 mL) was added Et₃N (32 μL, 0.23 mmol). The mixture was warmed to room temperature and stirred for 1.5 h. Sat. aq. NaHCO₃, water and EtOAc were added. The aqueous layer was extracted with EtOAc (4×) and the combined organic extracts were washed with brine (1×), dried (MgSO₄) and concentrated. The residue thus obtained was purified by column chromatography eluting with 30% EtOAc/hexanes→50% EtOAc/hexanes to afford the title compound.

¹H NMR δ (ppm)(Acetone): 7.80 (3H, d, J=8.46 Hz), 7.66 (2H, d, J=8.47 Hz), 5.22 (1H, s), 4.11-4.04 (1H, m), 3.77 (3H, s), 3.66 (1H, d, J=17.68 Hz), 3.52 (1H, d, J=17.69 Hz), 3.23-3.10 (3H, m), 3.15-3.07 (2H, m), 2.90 (1H, d, J=11.9 Hz), 1.95-1.85 (2H, m), 1.58-1.48 (6H, m).

MS (+ESI): 567.9, 569.9 [M+1]⁺

### Step 8: (3R,6S,8R)-3-Acetyl-8-(4-bromophenyl)-6-(2-methylprop-2-en-1-yl)-8-(trifluoromethyl)-1-thia-4,7-diazacycloundec-9-yn-5-one

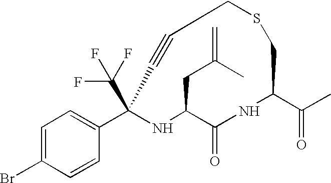

To a stirred, cold (0° C.) solution of (3R,6S,8R)-8-(4-bromophenyl)-6-(2-fluoro-2-methylpropyl)-N-methoxy-N-methyl-5-oxo-8-(trifluoromethyl)-1-thia-4,7-diazacycloundec-9-yne-3-carboxamide from Step 7, Example 7 (16.3 mg, 0.03 mmol) in THF (0.5 mL) was added MeMgBr (3M in Et₂O, 200 μL, 0.6 mmol). After stirring at 0° C. for 1 h, the reaction was warmed to room temperature and stirred for an additional 1 h. Sat. aq. NH₄Cl was added and the aqueous layer was extracted with EtOAc (3×). The combined organics were washed with brine (1×), dried (MgSO₄) and concentrated. The residue thus obtained was purified by column chromatography eluting with 30% EtOAc/hexanes to afford the title compound.

¹H NMR δ (ppm)(CDCl₃): 7.61 (2H, d, J=8.45 Hz), 7.48 (2H, d, J=8.38 Hz), 6.78 (1H, br d), 4.86 (1H, s), 4.80 (1H, s), 4.79-4.76 (1H, m), 3.75 (1H, t), 3.48 (1H, d, J=17.36 Hz), 3.43 (1H, dd), 3.26 (1H, d, J=17.40 Hz), 3.15 (1H, dd), 2.31-2.28 (2H, m), 2.19 (3H, s), 1.82 (3H, s).

MS (+ESI): 503, 505 [M+1]⁺

### Example 8

### Synthesis of (3R,6S,8R)-6-(2-fluoro-2-methylpropyl)-5-oxo-8-(4′-piperazin-4-ium-1-ylbiphenyl-4-yl)-8-(trifluoromethyl)-1-thia-4,7-diazacycloundec-9-yne-3-carboxylate

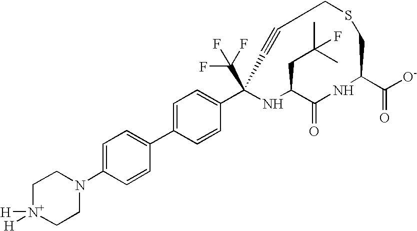

### Step 1: Methyl (3R,6S,8R)-8-{4′-[4-(tert-butoxycarbonyl)piperazin-1-yl]biphenyl-4-yl}-6-(2-fluoro-2-methylpropyl)-5-oxo-8-(trifluoromethyl)-1-thia-4,7-diazacycloundec-9-yne-3-carboxylate

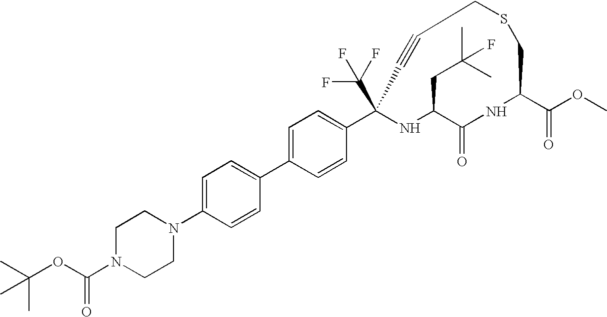

A vial containing methyl (3R,6S,8R)-8-(4-bromophenyl)-6-(2-fluoro-2-methylpropyl)-5-oxo-8-(trifluoromethyl)-1-thia-4,7-diazacycloundec-9-yne-3-carboxylate from Step 5, Example 7 (11 mg, 0.02 mmol), {4-[4-(tert-butoxycarbonyl)piperazin-1-yl]phenyl}boronic acid (J. Org. Chem. 2003, 68, 2633; 9.4 mg, 0.031 mmol), PdCl₂(dppf) (2 mg, 0.002 mmol) was flushed with nitrogen followed by the addition of DMF (0.4 mL) and Na₂CO₃ (2M aq., 31 μL, 0.061 mmol). The mixture was heated to 90° C. and the reaction was stirred for overnight. EtOAc and water were added and the aqueous layer was extracted with EtOAc (3×). The combined organics were washed with brine (1×), dried (MgSO₄) and concentrated. The residue thus obtained was purified by column chromatography on silica gel eluting with 20% EtOAc/hexanes→50% EtOAc/hexanes to afford the title compound.

¹H NMR δ (ppm)(Acetone): 8.02 (111, d, J=8.15 Hz), 7.83 (21-1, d, J=8.38 Hz), 7.66 (21-1, d, J=8.12 Hz), 7.59 (2H, d, J=8.49 Hz), 7.08 (2H, d, J=8.44 Hz), 4.91-4.83 (1H, m), 4.11-4.06 (1H, m), 3.74-3.70 (4H, m), 3.55 (4H, br s), 3.47 (1H, d, J=17.82 Hz), 3.28 (1H, d, J=15.22 Hz), 3.23-3.14 (5H, m), 2.94-2.91 (1H, m), 1.95-1.91 (2H, m), 1.58-1.52 (6H, m), 1.45 (9H, s).

MS (+ESI): 721.6 [M+1]⁺

### Step 2: (3R,6S,8R)-8-{4′-[4-(tert-Butoxycarbonyl)piperazin-1-yl]biphenyl-4-yl}-6-(2-fluoro-2-methylpropyl)-8-methyl-5-oxo-1-thia-4,7-diazacycloundec-9-yne-3-carboxylic acid

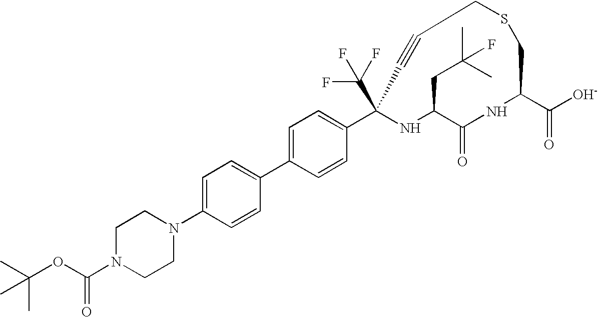

To a solution of methyl (3R,6S,8R)-8-{4′-[4-(tert-butoxycarbonyl)piperazin-1-yl]biphenyl-4-yl}-6-(2-fluoro-2-methylpropyl)-5-oxo-8-(trifluoromethyl)-1-thia-4,7-diazacycloundec-9-yne-3-carboxylate from Step 1, Example 8 (3 mg, 0.004 mmol) in DME (0.3 mL) and MeOH (0.1 mL) was added a 2M aqueous solution of LiOH (0.01 mL, 0.02 mmol). The resultant suspension was stirred at room temperature for 2.5 h. The mixture was acidified with 10% aq. HCl and the aqueous layer was extracted with EtOAc (3×). The combined organics were washed with brine (1×), dried (MgSO₄) and concentrated. The residue thus obtained was purified by column chromatography eluting with 60% EtOAc/hexanes→59% EtOAc/hexanes/1% acetic acid to afford the title compound.

¹H NMR δ (ppm)(Acetone): 7.87 (3H, d, J=8.07 Hz), 7.69 (2H, d, J=8.16 Hz), 7.62 (21-1, d, J=8.30 Hz), 7.11 (2H, d, J=8.37 Hz), 4.76-4.72 (1H, m), 4.14-4.07 (1H, m), 3.70 (1H, d, J=−17.57 Hz), 3.59 (4H, br s), 3.47 (1H, d, J=17.52 Hz), 3.43-3.37 (1H, m), 3.26-3.17 (5H, m), 2.95-2.89 (1H, m), 2.02-1.94 (2H, m), 1.60-1.54 (6H, m), 1.49 (9H, s).

MS (+ESI): 707.2 [M+1]⁺

### Step 3: (3R,6S,8R)-6-(2-Fluoro-2-methylpropyl)-5-oxo-8-(4′-piperazin-4-ium-1-ylbiphenyl-4-yl)-8-(trifluoromethyl)-1-thia-4,7-diazacycloundec-9-yne-3-carboxylate

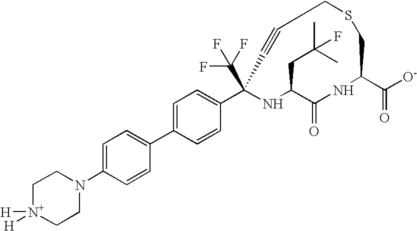

To a stirred solution of (3R,6S,8R)-8-{4′-[4-(tert-butoxycarbonyl)piperazin-1-yl]biphenyl-4-yl}-6-(2-fluoro-2-methylpropyl)-8-methyl-5-oxo-1-thia-4,7-diazacycloundec-9-yne-3-carboxylic acid from Step 2, Example 8 (2.3 mg, 0.003 mmol) in CH₂Cl₂ (0.16 mL) was added TFA (0.16 mL). The reaction was stirred at room temperature for 0.5 h. The solvent was removed by rotary evaporation (TFA was azeotroped off with heptane) to provide the title compound.

¹H NMR δ (ppm) (DMSO): 8.82-8.75 (3H, m), 7.78-7.68 (4H, m), 7.63 (2H, d, J=8.31 Hz), 7.11 (2H, d, J=8.38 Hz), 4.66-4.61 (1H, m), 4.04-3.95 (1H, m), 3.72 (1H, d, J=17.7 Hz), 3.54 (1H, d, J=17.7 Hz), 3.42 (411, br s), 3.17 (411, br s), 3.17-3.04 (2H, m), 2.84 (1H, d, J=12.08 Hz), 1.86-1.75 (2H, m), 1.55-1.49 (6H, m).

MS (+ESI): 607.2 [M+1]⁺

### Example 9

### Synthesis of 4S,7S,9R)-9-(4-Bromophenyl)-7-isobutyl-2,6-dioxo-9-trifluoromethyl)-1-oxa-5,8-diazacyclododec-10-yne-4-carbonitrile

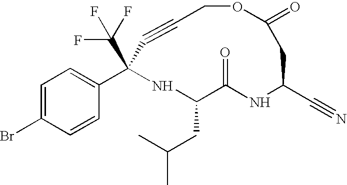

### Step 1: N-[(1R)-1-(4-Bromophenyl)-4-hydroxy-1-(trifluoromethyl)but-2-yn-1-yl]-L-leucine

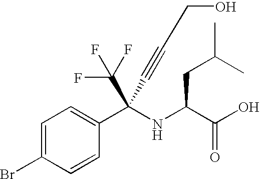

The title compound was prepared in a similar manner as described for Example 3, steps 1 to 4 from 1-(4-bromophenyl)-2,2,2-trifluoroethanone and (2S)-2-amino-4-methylpentan-1-ol.

### Step 2: tert-Butyl L-α-asparaginate

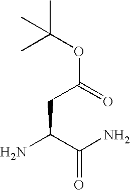

The title compound was prepared in a similar manner as described for Example 2, steps 1 to 2 from N-[(9H-fluoren-9-ylmethoxy)carbonyl]-L-aspartic acid, β-tert-butyl ester.

### Step 3: tert-Butyl N-[(1R)-1-(4-bromophenyl)-4-hydroxy-1-(trifluoromethyl)but-2-yn-1-yl]-L-leucyl-L-α-asparaginate

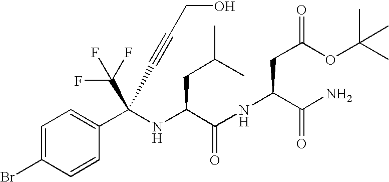

The title compound was prepared according to the procedure described for Example 3, step 5 from N-[(1R)-1-(4-bromophenyl)-4-hydroxy-1-(trifluoromethyl)but-2-yn-1-yl]-L-leucine and tert-butyl L-α-asparaginate.

¹H NMR (500 MHz, acetone-d₆) δ 7.80 (d, 2H), 7.70 (d, 1H), 7.58 (d, 2H), 6.76 (br s, 1H), 6.50 (br s 1H), 4.55 (m, 2H), 4.38 (d, 2H), 3.55 (m, 1H), 3.00 (m, 1H), 2.56 (m, 2H), 1.92 (m, 1H), 1.56 (m, 2H), 1.42 (s, 9H), 0.95 (m, 6H).

### Step 4: 4S,7S,9R)-9-(4-Bromophenyl)-7-isobutyl-2,6-dioxo-9-(trifluoromethyl)-1-oxa-5,8-diazacyclododec-10-yne-4-carboxamide

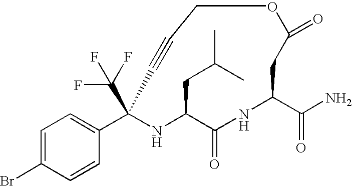

To a mixture of tent-butyl N-[(1R)-1-(4-bromophenyl)-4-hydroxy-1-(trifluoromethyl)but-2-yn-1-yl]-L-leucyl-L-α-asparaginate (320 mg, 0.54 mmol) in CH₂Cl₂ was added TFA (2 mL). The mixture was stirred at room temperature for 3 h. Volatile material was removed in vacuo. Crude NMR showed ˜80% of the tert-butyl ester was cleaved. This material was used directly for the cyclization reaction.

To the crude acid from above in THF (10 mL) was added Et₃N (130 μL, 0.9 mmol), followed by 2,4,6-trichlorobenzoyl chloride (120 μL, 0.77 mmol), and the mixture was stirred at room temperature for 2 h. The mixture was then diluted with toluene (30 mL) and was added dropwise (via syringe pump) over 3 h to a refluxing solution of DMAP (1.2 g, 9.8 mmol) in toluene (500 mL). The mixture was further stirred for 1 h. After cooling, solvent was removed in vacuo and the residue was diluted with water and extracted with EtOAc. The EtOAc extracts were washed successively with ˜0.5 N HCl, diluted brine, ˜0.2 N NaOH and brine; dried (Na₂SO₄) and concentrated. Chromatography over silica gel and elution with hexanes/EtOAc (1:3) gave 100 mg of a white powder which was triturated with Et₂O and hexanes (˜1:1) to afford the title compound.

¹H NMR (500 MHz, acetone-d₆): δ 7.79 (m, 3H), 7.64 (d, 2H), 6.76 (s, 1H), 6.53 (s, 1H), 5.10 (m, 1H), 4.99-4.88 (m, 2H), 3.84 (m, 1H), 2.87 (m, 2H), 2.55 (t, 1H), 2.08 (m, 1H), 1.40 (m, 2H), 0.94 (d, 6H).

MS (+ESI) m/z 518, 520 (MH⁺).

### Step 5: (4S,7S,9R)-9-(4-Bromophenyl)-7-isobutyl-2,6-dioxo-9-(trifluoromethyl)-1-oxa-5,8-diazacyclododec-10-yne-4-carbonitrile

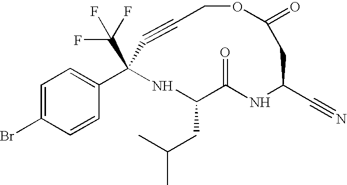

To a solution of (4S,7S,9R)-9-(4-bromophenyl)-7-isobutyl-2,6-dioxo-9-(trifluoromethyl)-1-oxa-5,8-diazacyclododec-10-yne-4-carboxamide (40 mg, 0.077 mmol) and pyridine (31 μL, 0.39 mmol) in dioxane (2 mL) at room temperature was added TFAA (22 μL, 0.15 mmol). The mixture was stirred for 30 min, quenched with sat. aq. NaHCO₃ and extracted with EtOAc. The EtOAc extract was washed with dilute brine, dried (Na₂SO₄) and concentrated to give the nitrile as a pale yellow foam.

¹H NMR (500 MHz, acetone-d₆): δ 8.30 (d, 1H), 7.78 (d, 2H), 7.64 (d, 2H), 5.66-5.59 (m, 1H), 5.08 (d, 1H), 4.86 (d, 1H), 3.75 (m, 1H), 3.21 (dd, 1H), 2.95-2.85 (m, 2H), 2.10 (m, 1H), 1.49-1.26 (m, 2H), 0.98-0.89 (m, 6H).

MS (+ESI): 500, 502 [M+1]⁺

### Example 10

### Synthesis of (3R,6S,8R)-3-acetyl-8-(4-bromophenyl)-6-(2-fluoro-2-methylpropyl)-8-(trifluoromethyl)-1-thia-4,7-diazacycloundec-9-yn-5-one

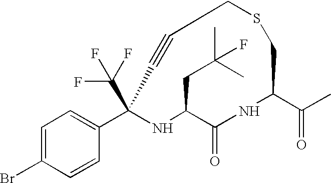

### Step 1: (3R,6S,8R)-8-(4-Bromophenyl)-6-(2-fluoro-2-methylpropyl)-3-(hydroxymethyl)-8-(trifluoromethyl)-1-thia-4,7-diazacycloundec-9-yn-5-one

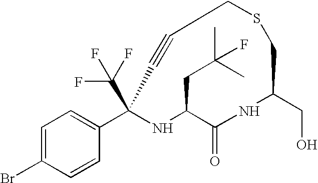

To a stirred, cold (0° C.) mixture of methyl (3R,6S,8R)-8-(4-bromophenyl)-6-(2-fluoro-2-methylpropyl)-5-oxo-8-(trifluoromethyl)-1-thia-4,7-diazacycloundec-9-yne-3-carboxylate from Step 5, Example 7 (385 mg, 0.714 mmol) in ethanol (10 mL) was added lithium chloride (121 mg, 2.86 mmol) followed by sodium borohydride (108 mg, 2.86 mmol) portion-wise. The mixture was warmed to room temperature and stirred for 2.5 hours. Water (2 drops) was added and the mixture was stirred for an additional 1.5 hour. Dilute aqueous ammonium chloride was added and the mixture was extracted twice with ethyl acetate. The combined organic layers were washed with brine and dried with magnesium sulfate. After removal of the solvent, the residue was purified by chromatography using ethyl acetate and hexanes (1.5:1) to afford the title compound (553 mg).

¹H NMR δ (ppm)(CD₃COCD₃): 7.8 (2H, m), 7.6 (2H, m), 7.5 (1H, NH), 4.2-4.1 (1H, m), 4.05-4.00 (1H, m), 3.95-3.85 (2H, m), 3.75-3.35 (4H, m), 3.1-3.0 (2H, m), 2.0-1.8 (2H, m), 1.55-1.45 (6H, m).

### Step 2: (3R,6S,8R)-8-4-Bromophenyl)-6-(2-fluoro-2-methylpropyl)-5-oxo-8-(trifluoromethyl)-1-thia-4,7-diazacycloundec-9-yne-3-carbaldehyde

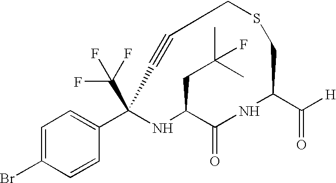

Dess-Martin periodinane (233 mg, 0.549 mmol) was added to a 0° C. solution of (3R,6S,8R)-8-(4-bromophenyl)-6-(2-fluoro-2-methylpropyl)-3-(hydroxymethyl)-8-(trifluoromethyl)-1-thia-4,7-diazacycloundec-9-yn-5-one (281 mg, 0.549 mmol) from Step 1, Example 10 in dichloromethane (6.1 mL) and the mixture was stirred at room temperature for 3 hours. The reaction mixture was diluted with dichloromethane and washed with dilute aqueous NaHCO₃ and brine. The organic layer was dried with magnesium sulfate and the solvent was removed in vacuo to afford the aldehyde which was used as such in the next step.

¹H NMR δ (ppm)(CD₃COCD₃): 9.6 (1H, CHO); contaminated with Dess-Martin residues.

### Step 3: (3R,6S,8R)-8-(4-Bromophenyl)-6-(2-fluoro-2-methylpropyl)-3-(1-hydroxyethyl)-8-(trifluoromethyl)-1-thia-4,7-diazacycloundec-9-yn-5-one

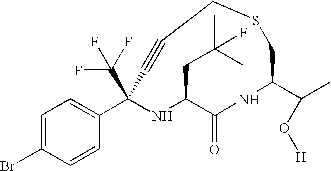

A THF (1 mL) solution of (3R,6S,8R)-8-(4-bromophenyl)-6-(2-fluoro-2-methylpropyl)-5-oxo-8-(trifluoromethyl)-1-thia-4,7-diazacycloundec-9-yne-3-carbaldehyde (179 mg, 0.351 mmol) from Step 2, Example 10 was added to a −20° C. mixture of 3M (THF) methylmagnesium chloride (0.351 mL) in THF-toluene (1:1, 6 mL). After stirring for 2 hours at −20° C., the mixture was diluted with dichloromethane and poured slowly on ice and dilute 1N HCl under vigorous stirring. The mixture was extracted twice with CH₂Cl₂, the organic layer was dried with magnesium sulfate and the solvent was removed in vacuo. A similar preparation was conducted using 117 mg of the aldehyde. Both batches were combined and purified on silica gel using ethyl acetate and hexanes (1:1) to afford the title compound (128 mg) as a mixture of isomers at the newly created asymmetric center.

¹H NMR δ (ppm)(CD₃COCD₃): 7.8 (2H, m), 7.65 (2H, m), 7.4 (1H, NH), 3.15 (2H, m), 1.60-1.45 (6H, m), 1.15 (3H, m); only well resolved resonances are reported)

### Step 4: (3R,6S,8R)-3-Acetyl-8-(4-bromophenyl)-6-(2-fluoro-2-methylpropyl)-8-(trifluoromethyl)-1-thia-4,7-diazacycloundec-9-yn-5-one

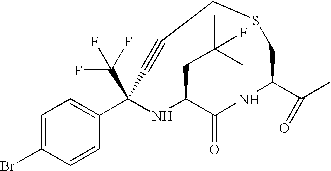

To a stirred, cold (0° C.) solution of (3R,6S,8R)-8-(4-bromophenyl)-6-(2-fluoro-2-methylpropyl)-3-(1-hydroxyethyl)-8-(trifluoromethyl)-1-thia-4,7-diazacycloundec-9-yn-5-one (118 mg, 0.225 mmol) from Step 3, Example 10 in dichloromethane (5 mL) was added Dess-Martin periodinane (105 mg, 0.247 mmol) and the mixture was stirred 2 hours at room temperature. The mixture was diluted with dichloromethane and washed successively with dilute aqueous sodium bicarbonate, sodium thiosulfate and brine. The organic layer was dried with magnesium sulfate and the solvent was removed in vacuo. The residue was purified on silica gel using ethyl acetate and hexanes (1:2) to afford the title compound (42.9 mg).

¹H NMR δ (ppm)(CD₃COCD₃): δ (1H, NH), 7.8 (2H, m), 7.7 (2H, m), 4.9 (1H, m), 4.1 (1H, m), 3.7 (1H, d), 3.45 (1H, d), 3.20-3.15 (1H, m), 3.1 (1H, m), 3.0-2.9 (1H, NH), 2.25 (3H, s), 2.0-1.8 (2H, m), 1.6-1.5 (6H, m).

MS (+ESI): 523.2, 525.2 [M+1]⁺

### Example 11

### Synthesis of (3R,6S,8R)-3-acetyl-8-(4-bromophenyl)-6-(2-fluoro-2-methylpropyl)-8-(trifluoromethyl)-1-thia-4,7-diazacycloundec-9-yn-5-one 1,1-dioxide

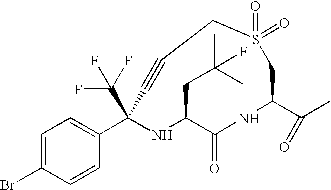

To a stirred, cold (0° C.) solution of (3R,6S,8R)-3-acetyl-8-(4-bromophenyl)-6-(2-fluoro-2-methylpropyl)-8-(trifluoromethyl)-1-thia-4,7-diazacycloundec-9-yn-5-one (41 mg, 0.078 mmol) from Step 4 of Example 10 in dichloromethane (1.5 mL) was added 76% m-chloroperoxybenzoic acid (39 mg, 0.172 mmol) and the mixture was stirred for 2 hours at room temperature. The mixture was cooled to 0° C. and powdered calcium hydroxide (17.3 mg, 0.234 mmol) was added. The suspension was stirred for 30 minutes and then filtered through celite. The filtrate was evaporated to dryness and applied to C-18 Lichroprep RP-18 gel and eluted using CH₃CN and water (3:2). Evaporation to dryness of the fractions containing the product yielded the title compound (37 mg).

¹H NMR δ (ppm)(CD₃COCD₃): 8.2 (1H, NH), 7.8-7.7 (2H, m), 7.65-7.60 (2H, m), 5.0 (1H, m), 4.4 (2H, m), 4.15-3.75 (3H, m), 3.05 (1H, NH), 2.25 (3H, s), 2.1-1.9 (2H, m), 1.6-1.4 (6H, m).

MS (−ESI): 554.7 [M−1]⁺

### Example 12

### Synthesis of (3R,6S,8R)-8-(1-benzothien-2-yl)-6-(2-fluoro-2-methylpropyl)-5-oxo-8-(trifluoromethyl)-1-thia-4,7-diazacycloundec-9-yne-3-carboxamide

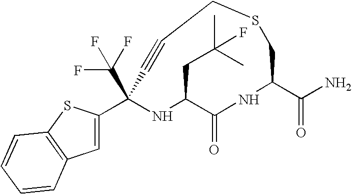

### Step 1: (4S)-2-(1-Benzothien-2-yl)-4-isobutyl-2-(trifluoromethyl)-1,3-oxazolidine

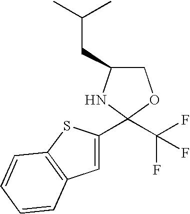

A suspension of 1-(1-benzothien-2-yl)-2,2,2-trifluoroethanone (9 g, 39.1 mmol), (S)-(+)-leucinol (5.09 mL, 39.1 mmol) and pyridinium p-toluenesulfonate (491 mg, 1.95 mmol) in toluene (98 mL) was heated in a 140° C. oil bath for 48 hours while water was collected in a Dean-Stark trap. The solvent was removed in vacuo and the residue was purified on silica gel using a gradient of ethyl acetate and hexanes (1:50 to 1:25) to afford a mixture of the starting material and a mixture of isomeric oxazolidines containing the desired (4S)-2-(1-benzothien-2-yl)-4-isobutyl-2-(trifluoromethyl)-1,3-oxazolidine (8.5 g total) which was used as such in the next step.

### Step 2: (2S)-2-{[(1R)-1-(1-Benzothien-2-yl)-4-{[tert-butyl(dimethyl)silyl]oxy}-1-(trifluoromethyl)but-2-yn-1-yl]amino}-4-methylpentan-1-ol

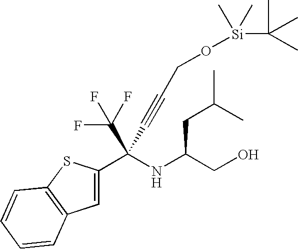

To a stirred, cold (−20° C.) solution of tert-butyl(dimethyl)(prop-2-yn-1-yloxy)silane (0.852 g, 53 mmol) in THF (5 mL) was added 2.5 M n-BuLi (2 mL, 5 mmol) and the mixture was stirred for 30 minutes. The mixture was then transferred using a canula into a −78° C. mixture of (4S)-2-(1-benzothien-2-yl)-4-isobutyl-2-(trifluoromethyl)-1,3-oxazolidine (0.329 g, 1 mmol) from Step 1, Example 12 and the mixture was slowly warmed to 0° C. After 30 minutes at this temperature, the mixture was poured on ice and dilute NH₄Cl and extracted twice with MTBE. The combined organic layers were washed with brine, dried with magnesium sulfate and the solvent was removed in vacuo. The residue was purified by chromatography on silica gel using ethyl acetate and hexanes (1:7) to afford the alcohol as a mixture of isomers which was used as such in the next step.

¹H NMR δ (ppm)(CD₃COCD₃): 4.6 (2H, CH₂OH); other resonances complex.

### Step 3: N-[(1R)-1-(1-Benzothien-2-yl)-4-{[tert-butyl(dimethyl)silyl]oxy}-1-(trifluoromethyl)but-2-yn-1-yl]-L-leucine

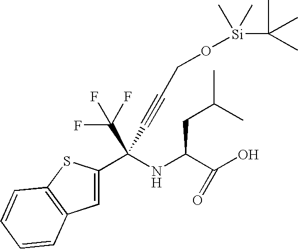

Oxalyl chloride (1.64 mL, 18.7 mmol) was added to a −78° C. mixture of DMSO (2.41 mL, 34 mmol) in dichloromethane (60 mL) and the mixture was reacted for 5 minutes. A dichloromethane (25 mL) solution of (2S)-2-{[(1R)-1-(1-benzothien-2-yl)-4-{[tert-butyl(dimethyl)silyl]oxy}-1-(trifluoromethyl)but-2-yn-1-yl]amino}-4-methylpentan-1-ol (8.5 g, 17 mmol) from Step 2, Example 12 was added and the mixture was reacted for 15 minutes. Triethylamine (11.95 mL, 85 mmol) was then added dropwise and the mixture was allowed to warm up slowly to room temperature. The mixture was poured on dilute aqueous hydrochloric acid and the organic layer was separated. The organic layer was washed with brine and dried with magnesium sulfate. Removal of the solvent yielded a residue which was purified on silica gel using ethyl acetate and hexanes (1:20) to afford a diastereomeric mixture (7.23 g) of aldehydes used as such in the next step.

¹H NMR δ (ppm)(CD₃COCD₃): 9.85 and 9.6 (1H, 2s, CHOs), 4.6 and 4.55 (2H, 2s. CH₂O).

To a stirred, cold (−5° C.) solution of aldehydes (7.23 g) from above in 1:1 THF-t-BuOH (180 mL) was added successively 2-methyl-2-butene (7.69 mL, 72.6 mmol), sodium dihydrogen phosphate (4.36 g, 36.3 mmol) and sodium chlorite (3.28 g, 36.3 mmol). The mixture was stirred at −5° C. for 45 minutes. The mixture was diluted with water and aqueous NH₄Cl and extracted twice with ethyl acetate. The combined organic layers were washed with brine, dried with magnesium sulfate and the solvent were removed in vacuo. The residue was used as such in the next step.

### Step 4: N-[(1R)-1-(1-Benzothien-2-yl)-4-hydroxy-1-(trifluoromethyl)but-2-yn-1-yl]-L-leucyl-S-trityl-D-cysteinamide

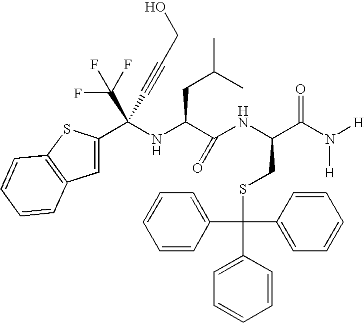

To a solution of S-trityl-L-cysteinamide from Step 2, Example 2 (2.15 g, 6.15 mmol) in DMF (12.3 mL) was added HATU (2.28 g, 6 mol), HOBt (676 mg, 5 mmol) and N-[(1R)-1-(1-benzothien-2-yl)-4-{[tert-butyl(dimethyl)silyl]oxy}-1-(trifluoromethyl)but-2-yn-1-yl]-L-leucine (2.57 g, 5 mmol) from Step 3, Example 12. The mixture was cooled to 0° C. and Et₃N (3.51 mL, 25 mmol) was added. The reaction was stirred at room temperature for 3 hours. Saturated aqueous NaHCO₃, EtOAc:MTBE and water were added and the organic layer was separated. The aqueous layer was further extracted with 1:1 EtOAc:MTBE. The combined organic layers were washed with brine, dried with magnesium sulfate and concentrated. The residue was purified by chromatography on silica gel eluting with ethyl acetate and hexanes (3:2) to afford the title compound (1.14 g) enriched in the desired isomer.

¹H NMR δ (ppm)(CD₃COCD₃): 6.85 and 6.6 (2H, NH₂), 3.65 (1H, m), 3.2 (1H, m), 0.95 (6H, m); other resonances complex.

### Step 5: N-[(1R)-1-(1-Benzothien-2-yl)-4-bromo-1-(trifluoromethyl)but-2-yn-1-yl]L-leucyl-S-trityl-D-cysteinamide

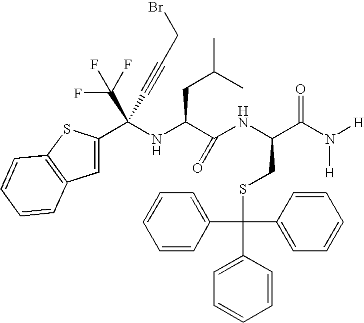

To a stirred, cold (0° C.) solution of triphenylphosphine (63.5 mg, 0.242 mmol) in dichloroethane (2 mL) was added bromine (11.44 uL, 0.222 mmol) and the mixture was stirred for 15 minutes. A dichloroethane (1 mL) solution of N-[(1R)-1-(1-benzothien-2-yl)-4-hydr oxy-1-(trifluoromethyDbut-2-yn-1-yl]-L-leucyl-5-trityl-D-cysteinamide (150 mg, 0.202 mmol) from Step 4, Example 12 was added. The mixture was reacted at 0° C. for 15 minutes and then at room temperature for 2 hours. The reaction was incomplete. An additional portion of the triphenylphosphine.bromine complex (prepared as above from triphenylphosphine (63.5 mg) and bromine (11.44 uL)) was added at 0° C. and the mixture was stirred for 2 hours at room temperature. The mixture was poured on ice and dilute NaHCO₃. It was extracted with dichloromethane (2×). The combined organic layers were washed with sodium thiosulfate, brine and dried with magnesium sulfate. After removal of the solvent, the residue was purified by chromatography on silica using ethyl acetate and hexanes (2:3) to afford the title product (100 mg).

¹H NMR δ (ppm)(CD3COCD₃): 7.90-7.85 (2H, m), 7.8 1H, s), 7.65 (1H, m), 7.5-7.2 (18H, m), 6.8 and 6.55 (2H, NH₂), 4.35 (1H, m), 4.25 (2H, m), 3.6 (1H, m), 3.2 (1H, NH), 2.60-2.45 (2H, m), 2.0-1.9 (1H, m), 1.6 (2H, m), 0.95 (6H, m).

### Step 6: (3R,6S,8R)-8-(1-Benzothien-2-yl)-6-(2-fluoro-2-methylpropyl)-5-oxo-8-(trifluoromethyl)-1-thia-4,7-diazacycloundec-9-yne-3-carboxamide

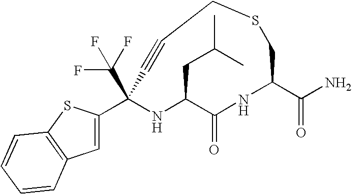

To a 0° C. solution of N-[(1R)-1-(1-benzothien-2-yl)-4-bromo-1-(trifluoromethyl)but-2-yn-1-yl]-L-leucyl-S-trityl-D-cysteinamide (100 mg, 0.124 mmol) from Step 5, Example 12 in dichloroethane (2 mL) was added triethylsilane (40 uL, 0.248 mmol) followed by trifluoroacetic acid (478 uL, 6.2 mmol). The mixture was stirred at room temperature for 15 minutes. The mixture was diluted with dichloroethane and heptane and the solvent were removed in vacuo (bath temperature <30° C.). This process was repeated twice. The residue was purified on silica gel using ethyl acetate and hexanes (2:1) to afford the intermediate mercapto bromide (52 mg). This intermediate was dissolved in DMF (2 mL) and the mixture was degassed using nitrogen gas. Potassium carbonate (50 mg, 0.362 mmol) was added and the mixture was stirred for 16 hours. The mixture was diluted with ethyl acetate and MTBE (1:1) and poured onto dilute aqueous ammonium chloride. The aqueous layer was extracted twice and the combined organic layers were washed with brine, dried with magnesium sulfate and the solvent removed in vacuo. The residue was passed on a short pad of silica gel using ethyl acetate and hexanes (2:1) to afford the title compound (3 mg).

¹H NMR δ (ppm)(CD₃COCD₃): 8.0-7.8 (4H, m), 7.45 (2H, m), 7.0 and 6.6 (2H, NH₂), 4.8 (1H, m), 4.0 (1H, m), 3.65 (1H, m), 3.45 (1H, m), 3.35 (1H, m), 3.1 (1H, m), 1.45 (2H, m), 1.0 (6H, m).

MS (+ESI): 484.0 [M+1]⁺

### Example 13

### Synthesis of (3R,6S,8R)-8-(1-benzothien-2-yl)-6-(2-fluoro-2-methylpropyl)-5-oxo-8-(trifluoromethyl)-1-thia-4,7-diazacycloundec-9-yne-3-carbonitrile

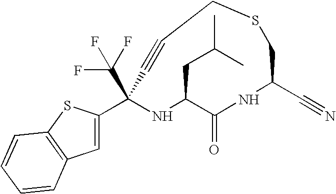

In a preparation of (3R,6S,8R)-8-(1-benzothien-2-yl)-6-(2-fluoro-2-methylpropyl)-5-oxo-8-(trifluoromethyl)-1-thia-4,7-diazacycloundec-9-yne-3-carboxamide following the procedure of Step 6, Example 12 but using 290 mg of mercapto bromide, the obtained cyclized intermediate was dissolved in 1,4-dioxane (10 mL) and the mixture was cooled to 0° C. Pyridine (208 uL), followed by trifluoroacetic anhydride (143 uL) were added and the mixture was warmed to room temperature. After 30 minutes, the mixture was poured on dilute aqueous sodium bicarbonate. The mixture was extracted twice with ethyl acetate and the combined organic layers were washed with brine and dried with magnesium sulfate. After removal of the solvent, the residue was purified on silica gel using ethyl acetate and hexanes (1:3) to afford the title compound (140 mg).

¹H NMR δ (ppm)(CD₃COCD₃): 8.85 (1H, m), 8.0 (1H, m), 7.9 (1H, m), 7.8 (1H, s), 7.45 (2H, m), 5.35 (1H, m), 3.9 (1H, m), 3.8 (1H, d), 3.45 (1H, d), 3.40-3.25 (2H, m), 3.05 (1H, m), 1.55-1.3 (2H, m), 1.0 (6H, m).

MS (+ESI): 466.0 [M+1]⁺

### Example 14

### Synthesis of (3S,5R,11S)-5-(4-bromophenyl)-3-(2-fluoro-2-methylpropyl)-11-(hydroxymethyl)5-(trifluoromethyl)-1,4-diazacycloundec-6-yn-2-one

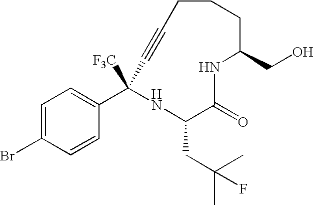

### Step 1: Benzyl (4S)-4-[(tert-butoxycarbonyl)amino]-5-hydroxypentanoate

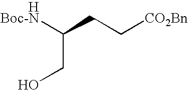

A stirred solution of (2S)-5-(benzyloxy)-2-[(tert-butoxycarbonyl)amino]-5-oxopentanoic acid (3.37 g, 10 mmol) and Et₃N (1.7 mL, 12.2 mmol) in THF (8 mL) was cooled to −23° C. Then a solution of isobutyl chloroformate (1.4 mL, 10.8 mmol) in THF (2 mL) was slowly added over 5 min (following procedure in J. Org. Chem., 1993, 58, 1586). The mixture was stirred at 0° C. for 1.5 hour. The white precipitate of triethylammonium chloride was filtered off and washed with THF (8 mL), and the combined filtrates and the washings were slowly added over 10 min to a solution of sodium borohydride (764 mg, 20 mmol) in water (8 mL) at −10° C. After the addition was complete, the reaction mixture was stirred at 0° C. for 1.5 h prior to acidification with 1N hydrochloric acid. The reaction mixture separated into two layers and the aqueous layer was extracted with EtOAc (3×50 mL). The combined organic extracts were washed with brine, dried over Na₂SO₄ and concentrated. The crude product was purified by column chromatography on silica gel eluting with a gradient of 40% EtOAc/hexanes→100% EtOAc/hexanes to give the title compound as a white solid

### Step 2: tert-Butyl (4S)-4-[3-(benzyloxy)-3-oxopropyl]-2,2-dimethyl-1,3-oxazolidine-3-carboxylate

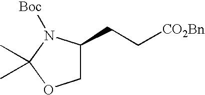

A catalytic amount of PPTS (790 mg, 3.1 mmol) was added to a solution of benzyl (4S)-4-[(tent-butoxycarbonyl)amino]-5-hydroxypentanoate from Step 1, Example 14 (11.56 g, 35.7 mmol) in a 9:1 mixture of dichloromethane (90 mL) and 2-methoxypropene (10 mL, 104 mmol). The reaction was stirred at room temperature for 1.5 h. Solvents were removed in vacuo, the resulting residue was taken up in EtOAc, washed with half-saturated NaHCO₃, dried over Na₂SO₄ and concentrated. The crude product was purified by column chromatography on silica gel eluting with 10% EtOAc/hexanes→15% EtOAc/hexanes→20% EtOAc/hexanes to give the title compound as a light-yellow oil.

### Step 3: 3-[(4S)-3-tert-Butoxycarbonyl)-2,2-dimethyl-1,3-oxazolidin-4-yl]propanoic acid

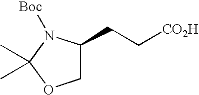

To an ice-cold solution of tent-butyl (4S)-4-[3-(benzyloxy)-3-oxopropyl]-2,2-dimethyl-1,3-oxazolidine-3-carboxylate from Step 2, Example 14 (11.73 g, 32.2 mmol) in THF (250 mL) and methanol (65 mL) was slowly added aqueous lithium hydroxide IN (40 mL, 40 mmol). The resulting reaction was allowed to proceed at room temperature for 18 h. Solvents were removed in vacuo, the resulting residue was taken up in EtOAc, washed with NH₄Cl, dried over Na₂SO₄ and concentrated to give the title compound as a light-yellow oil.

### Step 4: tert-Butyl (4S)-4-(3-hydroxypropyl)-2,2-dimethyl-1,3-oxazolidine-3-carboxylate

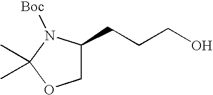

A stirred solution of 3-[(4S)-3-(tert-butoxycarbonyl)-2,2-dimethyl-1,3-oxazolidin-4-yl]propanoic acid from Step 3, Example 14 (12.42 g, 32 mmol) and Et₃N (6.0 mL, 43 mmol) in THF (26 mL) was cooled to −23° C. Then a solution of isobutyl chloroformate (5.0 mL, 10.8 mmol) in THF (6.5 mL) was slowly added over 10 min (following procedure in J. Org. Chem., 1993, 58, 1586). The mixture was stirred at 0° C. for 1.5 h. The white precipitate of triethylammonium chloride was filtered off and washed with THF (25 mL), and the combined filtrates and the washings were slowly added over 10 min to a solution of sodium borohydride (2.33 g, 61.6 mmol) in water (26 mL) at −10° C. After the addition was complete, the reaction mixture was stirred at 0° C. for 1.5 h and at room temperature for 16 h prior to acidification with 1N hydrochloric acid. The reaction mixture separated into two layers, the aqueous layer was extracted with EtOAc (3×150 mL). The combined organic extracts were washed with brine, dried over Na₂SO₄ and concentrated. The crude product was purified by column chromatography on silica gel eluting with 50% EtOAc/hexanes→60% EtOAc/hexanes→70% EtOAc/hexanes to give the title compound as a colourless oil.

¹H NMR (500 MHz, d₆-acetone) δ 3.92 (1H, m), 3.88-3.74 (1H, br m), 3.71 (1H, d), 3.57-3.47 (3H, m), 1.87-1.70 (1H, br m), 1.60-1.37 (18H, m+2s).

### Step 5: tert-Butyl (4S)-4-(3-bromopropyl)-2,2-dimethyl-1,3-oxazolidine-3-carboxylate

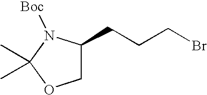

To an ice-cold solution of triphenylphosphine (7.6 g, 29 mmol) in THF (200 mL) was added bromine dropwise (1.5 mL, 29 mmol). The resulting light-yellow slurry was stirred for 30 min. A cold (0° C.) solution of tert-butyl (4S)-4-(3-hydroxypropyl)-2,2-dimethyl-1,3-oxazolidine-3-carboxylate from Step 4, Example 14 (3.76 g, 14.5 mmol) in THF (45 mL) was slowly added to the triphenylphosphine bromine suspension. Upon completion of addition, the resulting mixture was stirred at room temperature for 30 min. The reaction mixture was diluted with Et₂O (200 mL), washed with aqueous sodium thiosulfate (2×100 mL), dried over Na₂SO₄ and concentrated. The crude product was purified by column chromatography on silica gel eluting with a gradient of 0% EtOAc/hexanes→30% EtOAc/hexanes to give the title compound as a colourless oil.

¹H NMR (500 MHz, d₆-acetone) δ 3.98 (1H, dd), 3.96-3.86 (1H, br d), 3.77 (1H, d), 3.56 (2H, m), 1.98-1.82 (3H, m), 1.77-1.66 (1H, br m), 1.54 (3H, s), 1.48 (9H, s), 1.46 (3H, s).

### Step 6: tert-Butyl (4S)-2,2-dimethyl-4-[5-(trimethylsilyl)pent-4-yn-1-yl]-1,3-oxazolidine-3-carboxylate

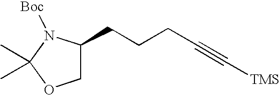

To a solution of ethynyltrimethylsilane (2.2 mL, 15.6 mmol) in THF (20 mL) at −78° C. was added a solution of butyllithium in hexane (2.4 M, 6.0 mL, 14.4 mmol). Of this solution, 15 mL (8.0 mmol) was transferred to a round bottom flask, HMPA (1.4 mL, 8.0 mmol) was added and the mixture was warmed to 0° C. for 30 min. To this mixture was added a solution of tert-butyl (4S)-4-(3-bromopropyl)-2,2-dimethyl-1,3-oxazolidine-3-carboxylate from Step 5, Example 14 (1.5 g, 4.65 mmol) in THF (15 mL). The reaction was stirred at 0° C. for 1 h and then at room temperature for 16 h. The reaction mixture was partitioned between EtOAc and NH₄OAc buffer, the organic layer was dried over Na₂SO₄ and concentrated. The crude product was purified by column chromatography on silica gel eluting with a gradient of 10% EtOAc/hexanes→30% EtOAc/hexanes to give the title compound as a colourless oil.

¹H NMR (600 MHz, d₆-acetone) δ 3.95 (1H, t), 3.90-3.82 (1H, br m), 3.72 (1H, d), 2.24 (2H, br m), 1.79 (1H, br s), 1.61 (1H, br s), 1.52-1.39 (2H, m), 1.51 (3H, s), 1.46 (9H, s), 1.42 (3H, s), 0.10 (9H, s).

### Step 7: tert-Butyl (4S)-2,2-dimethyl-4-pent-4-yn-1-yl-1,3-oxazolidine-3-carboxylate

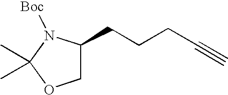

To an ice-cold solution of tert-butyl (4S)-2,2-dimethyl-4-[5-(trimethylsilyppent-4-yn-1-yl]-1,3-oxazolidine-3-carboxylate from Step 6, Example 14 (938 mg, 2.8 mmol) in THF (30 mL) and methanol (0.6 mL) was added TBAF (1M in THF, 1.4 mL, 1.4 mmol). The reaction was stirred at room temperature for 18 h. The reaction mixture was partitioned between EtOAc and NH₄OAc buffer, the organic layer was dried over Na₂SO₄ and concentrated. The crude product was purified by column chromatography on silica gel eluting with a gradient of 10% EtOAc/hexanes→30% EtOAc/hexanes to give the title compound as a colourless oil.

¹H NMR (600 MHz, d₆-acetone) δ 3.95 (1H, t), 3.89-3.80 (1H, br m), 3.73 (1H, d), 2.43 (1H, br d), 2.21 (2H, br s), 1.80 (1H, m), 1.62 (1H, br m), 1.58-1.42 (2H, m), 1.52 (3H, s), 1.46 (9H, s), 1.42 (3H, s).

### Step 8: tert-Butyl (4S)-4-((6R)-6-(4-bromophenyl)-7,7,7-trifluoro-6-{[(1S)-3-fluoro-1-(hydroxymethyl)-3-methylbutyl]amino}hept-4-yn-1-yl)-2,2-dimethyl-1,3-oxazolidine-3-carboxy late

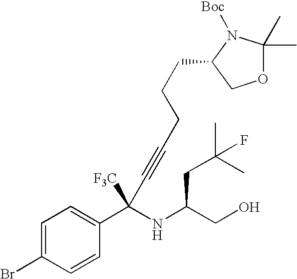

To a solution of tert-butyl (49-2,2-dimethyl-4-pent-4-yn-1-yl-1,3-oxazolidine-3-carboxylate from Step 7, Example 14 (90 mg, 0.34 mmol) in THF (1 mL) at −78° C. was added a solution of butyllithium in hexane (2.4 M, 0.14 mL, 0.34 mmol). This solution was stirred at −20° C. for 30 min and then cooled back to −78° C. To this mixture was added a solution of (2R,4S)-2-(4-bromophenyl)-4-(2-fluoro-2-methylpropyl)-2-(trifluoromethyl)-1,3-oxazolidine from step 1, Example 3 (75 mg, 0.20 mmol) in THF (0.8 mL). The reaction was stirred at −10° C. for 2 h. The reaction mixture was partitioned between Et₂O and NH₄OAc buffer, the organic layer was dried over Na₂SO₄ and concentrated. The crude product was purified by column chromatography on silica gel eluting with a gradient of 10% EtOAc/hexanes→50% EtOAc/hexanes to give the title compound as a colourless oil.

¹H NMR (500 MHz, d₆-acetone) δ 7.85 (21-1, d), 7.60 (2H, m), 3.96 (1H, m), 3.89 (1H, m), 3.74 (1H, d), 3.48 (1H, m), 3.33 (1H, m), 3.17 (1H, m), 3.09 (1H, m), 2.52 (2H, t), 1.95 (1H, m), 1.66 (3H, br m), 1.52-1.34 (22H, m), 1.20 (1H, dt).

MS (+ESI): 637.3, 639.4 [MH]⁺

### Step 9: tert-Butyl (4S)-4-((6R)-6-(4-bromophenyl)-7,7,7-trifluoro-6-{[(1S)-3-fluoro-1-formyl-3-methylbutyl]amino}hept-4-yn-1-yl)-2,2-dimethyl-1,3-oxazolidine-3-carboxylate

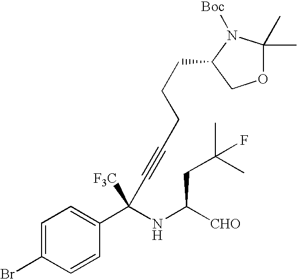

Oxalyl chloride (0.031 mL, 0.36 mmol) was added to a −78° C. mixture of DMSO (0.031 mL, 0.44 mmol) in dichloromethane (0.8 mL) and the mixture was reacted for 10 minutes. A solution of tert-butyl (4S)-4-(6R)-6-(4-bromophenyl)-7,7,7-trifluoro-6-{[(1S)-3-fluoro-1-(hydroxymethyl)-3-methylbutyl]amino}hept-4-yn-1-yl)-2,2-dimethyl-1,3-oxazolidine-3-carboxylate from Step 8, Example 14 (128 mg, 0.20 mmol) in dichloromethane (1 mL) was added and the mixture was allowed to react for 15 minutes. Triethylamine (0.125 mL, 0.9 mmol) was then added dropwise and the mixture was allowed to warm up slowly to 0° C. (2 h). The reaction mixture was partitioned between EtOAc and water, the organic layer was dried over Na₂SO₄ and concentrated. The crude product was purified by column chromatography on silica gel eluting with a gradient of 10% EtOAc/hexanes→30% EtOAc/hexanes to give the title compound as a colourless oil.

### Step 10: N-[(1R)-1-(4-Bromophenyl)-6-[(4S)-3-(tert-butoxycarbonyl)-2,2-dimethyl-1,3-oxazolidin-4-yl]-1-(trifluoromethyl)hex-2-yn-1-yl]-4-fluoro-L-leucine

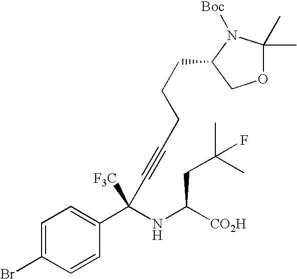

tert-Butyl(4S)-4-((6R)-6-(4-bromophenyl)-7,7,7-trifluoro-6-{[(1S)-3-fluoro-1-formyl-3-methylbutyl]amino}hept-4-yn-1-yl)-2,2-dimethyl-1,3-oxazolidine-3-carboxylate from Step 9, Example 14 (84 mg, 0.13 mmol) was dissolved in t-BuOH (0.6 mL) and water (0.3 mL) and cooled to 0° C. To this solution was added 2-methylbut-1-ene (0.085 mL, 0.80 mmol) followed by H₃PO₄ (1.2 M in water, 0.33 mL, 0.40 mmol) and NaClO₂ (1 M in water, 0.33 mL, 0.33 mmol). The reaction was warmed to room temperature and stirred for 1.5 h. Water and EtOAc were added and the aqueous layer was extracted with EtOAc (3×). The combined organics were washed with brine (1×), dried (Na₂SO₄) and concentrated. The crude product was purified by column chromatography on silica gel eluting with EtOAc/hexanes/AcOH (10:90:0)→(30:70:0)→(30:70:1) to give the title compound as a colourless oil.

¹H NMR (500 MHz, d₆-acetone) δ 10.97 (1H, br s), 7.78 (2H, m), 7.62 (2H, m), 3.97-3.82 (3H, m), 3.76 (1H, d), 2.88 (1H, br t), 2.43 (2H, br m), 1.95-1.2 (27H, complex m), 0.87 (3H, m).

### Step 11: N-[(1R,7S)-7-Amino-1-(4-bromophenyl)-8-hydroxy-1-(trifluoromethyl)oct-2-yn-1-yl]-4-fluoro-L-leucine

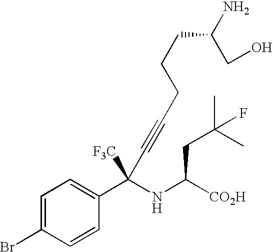

To a solution of N—R[(1R)-1-(4-bromophenyl)-6-[(4S)-3-(tert-butoxycarbonyl)-2,2-dimethyl-1,3-oxazolidin-4-yl]-1-(trifluoromethyphex-2-yn-1-yl]-4-fluoro-L-leucine from Step 10, Example 14 (67 mg, 0.10 mmol) in dichloromethane (0.5 mL) at 0° C. was added trifluoroacetic acid (0.20 mL, 2.6 mmol). This solution was stirred at room temperature for 1 h. The solvent was removed under reduced pressure and the resulting residue was co-evaporated twice with heptane to give the title compound as a yellow oil.

MS (+ESI): 510.7, 512.7 [MH]⁺

### Step 12: (3S,5R,11S)-5-(4-Bromophenyl)-3-(2-fluoro-2-methylpropyl)-11-(hydroxymethyl)-5-(trifluoromethyl)-1,4-diazacycloundec-6-yn-2-one

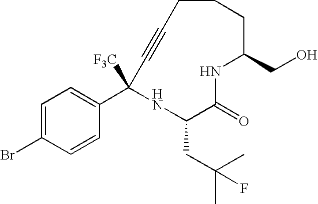

To a solution of HATU (142 mg, 0.37 mmol) in DMF (2.0 mL) at 0° C. was added triethylamine (0.10 mL, 0.72 mmol). To this ice-cold solution was added a solution of N-[(1R,7S)-7-amino-1-(4-bromophenyl)-8-hydroxy-1-(trifluoromethyl)oct-2-yn-1-yl]-4-fluoro-L-leucine from Step 11, Example 14 (64 mg, 0.1 mmol) in DMF (1 mL) over 30 min using a syringe pump. The resulting mixture was stirred at room temperature for 4 h. The reaction mixture was partitioned between EtOAc and half-saturated NaHCO₃, the organic layer was dried over Na₂SO₄ and concentrated. The crude product was purified by column chromatography on silica gel eluting with a gradient of 30% EtOAc/hexanes→100% EtOAc/hexanes to give the title compound.

¹H NMR (500 MHz, d₆-acetone) δ 7.77 (2H, d), 7.62 (2H, d), 7.42 (1H, br d), 3.97 (1H, m), 3.85 (1H, m), 3.80 (1H, m), 3.5 (2H, m), 2.75 (1H, d), 2.44 (2H, m), 2.01 (1H, m), 1.91-1.28 (3H, m), 1.63 (2H, m), 1.48 (6H, dd).

MS (+ESI): 492.7, 494.7 [MH]⁺

### Example 15

### Synthesis of (2S,5S,11R)-11-(4-bromophenyl)-2-(2-fluoro-2-methylpropyl)-3-oxo-11-(trifluoromethyl)-1,4-diazacycloundec-9-yne-5-carboxylic acid

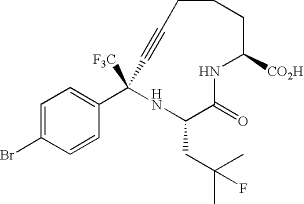

### Step 1: (2S,5S,11R)-11-(4-Bromophenyl)-2-(2-fluoro-2-methylpropyl)-3-oxo-11-(trifluoromethyl)-1,4-diazacycloundec-9-yne-5-carbaldehyde

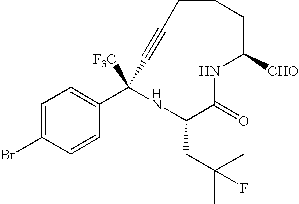

Oxalyl chloride (0.013 mL, 0.14 mmol) was added to a −78° C. mixture of DMSO (0.013 mL, 0.18 mmol) in dichloromethane (0.8 mL) and the mixture was stirred for 10 minutes. A solution of (3S,5R,11S)-5-(4-bromophenyl)-3-(2-fluoro-2-methylpropyl)-11-(hydroxymethyl)-5-(trifluoromethyl)-1,4-diazacycloundec-6-yn-2-one from Step 12, Example 14 (20 mg, 0.04 mmol) in dichloromethane (1 mL) was added and the mixture was allowed to react for 15 minutes. Triethylamine (0.05 mL, 0.36 mmol) was then added and the mixture was allowed to warm up slowly to 0° C. (2 h). The reaction mixture was partitioned between EtOAc and water, the organic layer was dried over Na₂SO₄ and concentrated. The crude product was purified by column chromatography on silica gel eluting with a gradient of 10% EtOAc/hexanes→50% EtOAc/hexanes to give the title compound.

¹H NMR (500 MHz, d₆-acetone) δ 9.67 (1H, s), 7.96 (1H, br d), 7.77 (2H, m), 7.62 (2H, m), 4.63 (1H, m), 4.03 (1H, m), 2.48 (2H, t), 2.22 (2H, m), 2.02-1.82 (3H, m), 1.72 (1H, m), 1.64 (1H, m), 1.51 (6H, dd).

MS (+ESI): 490.8, 492.8 [MH]⁺

### Step 2: (2S,5S,11R)-11-(4-Bromophenyl)-2-(2-fluoro-2-methylpropyl)-3-oxo-11-(trifluoromethyl)-1,4-diazacycloundec-9-yne-5-carboxylic acid

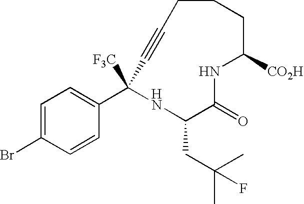

(2S,5S,11R)-11-(4-Bromophenyl)-2-(2-fluoro-2-methylpropyl)-3-oxo-11-(trifluoromethyl)-1,4-diazacycloundec-9-yne-5-carbaldehyde from Step 1, Example 15 (6 mg, 0.13 mmol) was dissolved in t-BuOH (0.3 mL) and water (0.15 mL) and cooled to 0° C. To this solution was added 2-methylbut-1-ene (0.012 mL, 0.113 mmol) followed by H₃PO₄ (1.2 M in water, 0.054 mL, 0.065 mmol) and NaClO₂ (1 M in water, 0.054 mL, 0.054 mmol). The reaction was warmed to room temperature and stirred for 1.5 h. Water and EtOAc were added and the aqueous layer was extracted with EtOAc (3×). The combined organics were washed with brine (1×), dried (Na₂SO₄) and concentrated. The crude product was purified by column chromatography on silica gel eluting with EtOAc/hexanes/AcOH (50:50:0)→(50:50:0.5) to give the title compound.

¹H NMR (500 MHz, d₆-acetone) δ 10.8 (1H, br s), 7.78 (3H, m), 7.62 (2H, d), 4.58 (1H, q), 4.06 (1H, q), 2.83 (1H, d), 2.50 (1H, dt), 2.40 (1H, m), 2.23 (1H, m), 2.02-1.79 (3H, m), 1.72 (2H, m), 1.48 (6H, dd).

MS (+ESI): 506.9, 508.9 [MH]⁺

### Example 16

### Synthesis of (2S,5S,11R)-11-(4-bromophenyl)-2-(2-fluoro-2-methylpropyl)-3-oxo-11-(trifluoromethyl)-1,4-diazacycloundec-9-yne-5-carboxamide

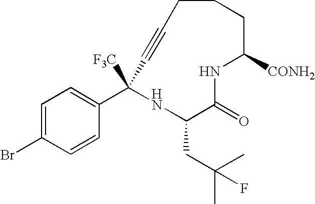

To an ice-cold solution of (2S,5S,11R)-11-(4-bromophenyl)-2-(2-fluoro-2-methylpropyl)-3-oxo-11-(trifluoromethyl)-1,4-diazacycloundec-9-yne-5-carboxylic acid from Step 2, Example 15 (10.6 mg, 0.02 mmol), HATU (12.1 mg, 0.03 mmol), 1-hydroxy-7-azabenzotriazole (2.4 mg, 0.018 mmol) and ammonium chloride (13 mg, 0.24 mmol) in DMF (1.0 mL) was added triethylamine (0.10 mL, 0.72 mmol). The resulting yellow solution was stirred at room temperature for 2.5 h. The reaction mixture was partitioned between EtOAc and half-saturated NaHCO₃, the organic layer was dried over Na₂SO₄ and concentrated. The crude product was purified by column chromatography on silica gel eluting with EtOAc/hexanes 50%→70%→100% to give the title compound.

¹H NMR (500 MHz, d₆-acetone) δ 7.78 (2H, d), 7.62 (3H, m), 7.00 (1H, br s), 7.52 (1H, br s), 4.47 (1H, m), 4.05 (1H, m), 2.50 (1H, dq), 2.38 (1H, m), 2.25 (1H, m), 1.98-1.62 (6H, m), 1.51 (6H, dd).

MS (+ESI): 505.8, 510.8 [MH]⁺

### Example 17

### Synthesis of (2S,5S,11R)-11-(4-bromophenyl)-2-(2-fluoro-2-methylpropyl)-3-oxo-11-(trifluoromethyl)-1,4-diazacycloundec-9-yne-5-carbonitrile

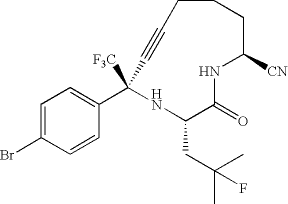

To an ice-cold solution of (2S,5S,11R)-11-(4-bromophenyl)-2-(2-fluoro-2-methylpropyl)-3-oxo-11-(trifluoromethyl)-1,4-diazacycloundec-9-yne-5-carboxamide from Example 16 (7.5 mg, 0.013 mmol) and pyridine (0.010 mL, 0.12 mmol) in dioxane (0.3 mL) was added trifluoroacetic anhydride (0.010 mL, 0.071 mmol). The resulting solution was stirred at room temperature for 0.5 h. The reaction mixture was partitioned between EtOAc and half-saturated NaHCO₃, the organic layer was dried over Na₂SO₄ and concentrated. The crude product was purified by column chromatography on silica gel eluting with EtOAc/hexanes 10%→30% to give the title compound.

¹H NMR (500 MHz, d₆-acetone) δ 8.43 (1H, br d), 7.78 (2H, d), 7.64 (2H, d), 5.10 (1H, m), 3.95 (1H, m), 2.58 (2H, m), 2.28 (1H, m), 2.05-1.82 (5H, m), 1.78 (1H, d), 1.52 (6H, t).

MS (+ESI): 487.8, 489.8 [MH]⁺

### Examples 18-29

The following compounds were prepared using methods analogous to those described in the preceding examples:

| Example | Structure | IUPAC Name | Characterization |
|---|---|---|---|
| 18 | 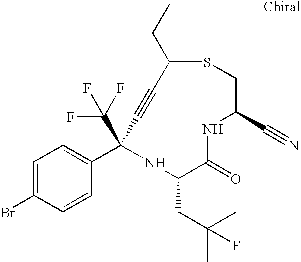 | (3R,6S,8R)-8-(4-bromophenyl)-11- ethyl-6-(2-fluoro-2-methylpropyl)-5- oxo-8-(trifluoromethyl)-1-thia-4,7- diazacycloundec-9-yne-3-carbonitrile | MS (+APCI): 533.8, 535.8 [M + 1]⁺ |
| 19 | 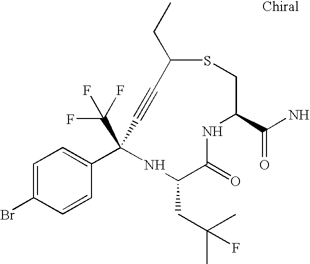 | (3R,6S,8R)-8-(4-bromophenyl)-11- ethyl-6-(2-fluoro-2-methylpropyl)-5- oxo-8-(trifluoromethyl)-1-thia-4,7- diazacycloundec-9-yne-3-carboxamide | MS (+ESI): 551.9, 553.9 [M + 1]⁺ |
| 20 | 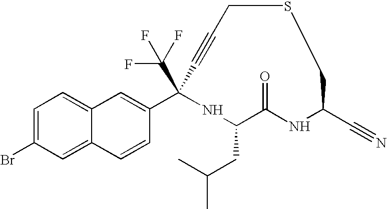 | (3R,6S,8R)-8-(6-bromo-2-naphthyl)-6- isobutyl-5-oxo-8-(trifluoromethyl)-1- thia-4,7-diazacycloundec-9-yne-3- carbonitrile | MS (+ESI): 538, 540 [M + 1]⁺ |
| 21 | 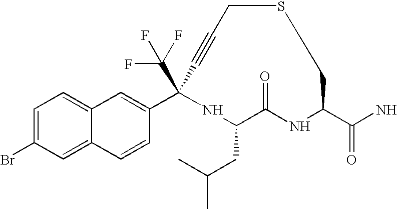 | (3R,6S,8R)-8-(6-bromo-2-naphthyl)-6- isobutyl-5-oxo-8-(trifluoromethyl)-1- thia-4,7-diazacycloundec-9-yne-3- carboxamide | MS (+ESI): 556, 558 [M + 1]⁺ |
| 22 | 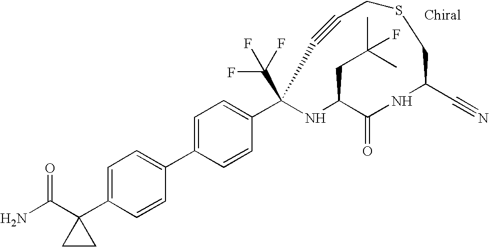 | 1-{4′-[(3R,6S,8R)-3-cyano-6-(2-fluoro- 2-methylpropyl)-5-oxo-8- (trifluoromethyl)-1-thia-4,7- diazacycloundec-9-yn-8-yl]biphenyl-4- yl}cyclopropanecarboxamide | MS (+ESI): 587.5 [M + 1]⁺ |
| 23 | 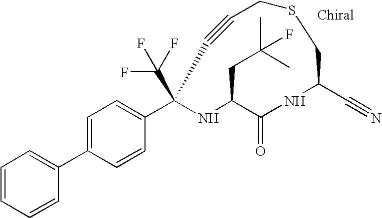 | (3R,6S,8R)-8-biphenyl-4-yl-6-(2-fluoro- 2-methylpropyl)-5-oxo-8- (trifluoromethyl)-1-thia-4,7- diazacycloundec-9-yne-3-carbonitrile | MS (+ESI): 504.5 [M + 1]⁺ |
| 24 | 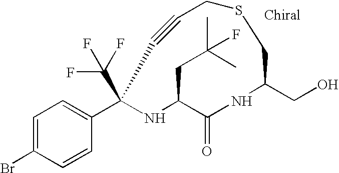 | (3R,6S,8R)-8-(4-bromophenyl)-6-(2- fluoro-2-methylpropyl)-3- (hydroxymethyl)-8-(trifluoromethyl)-1- thia-4,7-diazacycloundec-9-yn-5-one | MS (+ESI): 510.5, 512.5 [M + 1]⁺ |
| 25 | 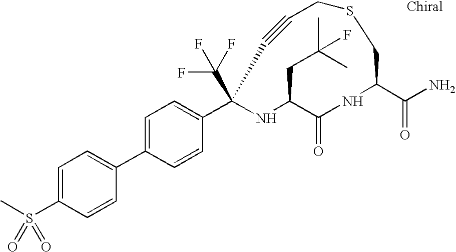 | (3R,6S,8R)-6-(2-fluoro-2-metylpropyl)- 8-[4′-(methylsulfonyl)biphenyl-4-yl]-5- oxo-8-(trifluoromethyl)-1-thia-4,7- diazacycloundec-9-yne-3-carboxamide | ¹H NMR δ (ppm) (Acetone): 8.05 (2 H, d, J = 8.16 Hz), 7.96 (4 H, d, J = 8.19 Hz), 7.86 (1 H, d, J = 8.24 Hz), 7.82 (2 H, d, J = 8.35 Hz), 6.99 (1 H, s), 6.60 (1 H, s), 4.75 (1 H, dd, J = 8.56, 4.36 Hz), 4.12 (1 H, s), 3.66 (1 H, d, J = 17.61 Hz), 3.45 (1 H, d, J = 17.62 Hz), 3.31 (1 H, dd, J = 14.60, 5.06 Hz), 3.17 (3 H, s), 3.10 (1 H, dd, J = 14.63, 3.73 Hz), 2.93 (1 H, d, J = 11.77 Hz), 2.05 (1 H, m), 1.93 (1 H, d, J = 28.40 Hz), 1.59-1.49 (6 H, m). |
| 26 | 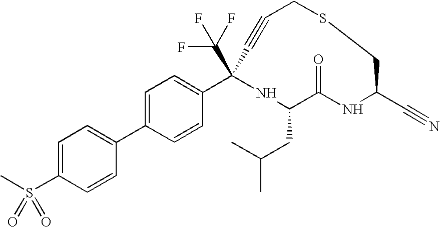 | (3R,6S,8R)-6-isobutyl-8-[4′- (methylsulfonyl)biphenyl-4-yl]-5-oxo- 8-(trifluoromethyl)-1-thia-4,7- diazacycloundec-9-yne-3-carbonitrile | MS (+APCI): 564.3 [M + 1]⁺ |
| 27 | 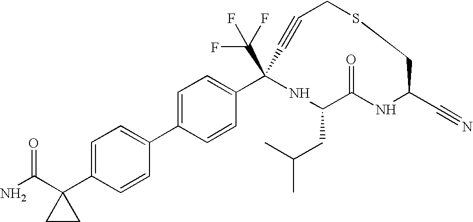 | 1-{4′-[(3R,6S,8R)-3-cyano-6-isobutyl- 5-oxo-8-(trifluoromethyl)-1-thia-4,7- diazacycloundec-9-yn-8-yl]biphenyl-4- yl}cyclopropanecarboxamide | MS (+APCI): 569.3 [M + 1]⁺ |
| 28 | 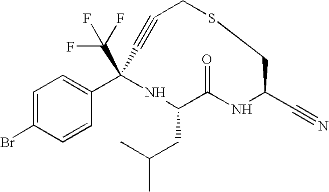 | (3R,6S,8R)-8-(4-bromophenyl)-6- isobutyl-5-oxo-8-(trifluoromethyl)-1- thia-4,7-diazacycloundec-9-yne-3- carbonitrile | MS (+APCI): 487.9, 489.9 [M + 1]⁺ |
| 29 | 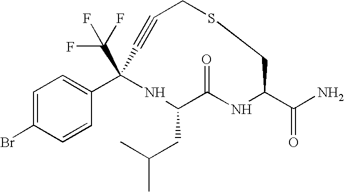 | (3R,6S,8R)-8-(4-bromophenyl)-6- isobutyl-5-oxo-8-(trifluoromethyl)-1- thia-4,7-diazacycloundec-9-yne-3- carboxamide | MS (+APCI): 506.2, 508.2 [M + 1]⁺ |

### Pharmaceutical Composition

As a specific embodiment of this invention, 100 mg of (3R,6S,8R)-8-(4-bromophenyl)-6-(2-fluoro-2-methylpropyl)-5-oxo-8-(trifluoromethyl)-1-thia-4,7-diazacycloundec-9-yne-3-carbonitrile 1,1-dioxide, is formulated with sufficient finely divided lactose to provide a total amount of 580 to 590 mg to fill a size 0, hard-gelatin capsule.

### Assays

The compounds disclosed in the present application exhibited activity in the following assays. In addition, the compounds disclosed in the present application have an enhanced pharmacological profile relative to previously disclosed compounds.

### Cathepsin K Assay

Serial dilutions (1/3) from 500 μM down to 0.0085 μM of test compounds were prepared in dimethyl sulfoxide (DMSO). Then 2 μL of DMSO from each dilution were added to 50 μL of assay buffer (MES, 50 mM (pH 5.5); EDTA, 2.5 mM; DTT, 2.5 mM and 10% DMSO) and 25 μL, of human cathepsin K (0.4 nM) in assay buffer solution. The assay solutions were mixed for 5-10 seconds on a shaker plate and incubated for 15 minutes at room temperature. Z-Leu-Arg-AMC (8 μM) in 25 μL of assay buffer was added to the assay solutions. Hydrolysis of the coumarin leaving group (AMC) was followed by spectrofluorometry (Exλ=355 nm; Emλ=460 nm) for 10 minutes. Percent of inhibition were calculated by fitting experimental values to standard mathematical model for dose response curve.

### Cathepsin L Assay

Serial dilutions (1/3) from 500 μM down to 0.0085 μM of test compounds were prepared in dimethyl sulfoxide (DMSO). Then 2 μL of DMSO from each dilution were added to 50 μL of assay buffer (MES, 50 mM (pH 5.5); EDTA, 2.5 mM; DTT, 2.5 mM and 10% DMSO) and 25 μL of human cathepsin L (0.5 nM) in assay buffer solution. The assay solutions were mixed for 5-10 seconds on a shaker plate and incubated for 15 minutes at room temperature. Z-Leu-Arg-AMC (8 uM) in 25 μL of assay buffer was added to the assay solutions. Hydrolysis of the coumarin leaving group (AMC) was followed by spectrofluorometry (Exλ=355 nm; Emλ=460 nm) for 10 minutes. Percent of inhibition were calculated by fitting experimental values to standard mathematical model for dose response curve.

### Cathepsin B Assay

Serial dilutions (1/3) from 500 μM down to 0.0085 μM of test compounds were prepared in dimethyl sulfoxide (DMSO). Then 2 μL of DMSO from each dilution were added to 50 μL of assay buffer (MES, 50 mM (pH 5.5); EDTA, 2.5 mM; DTT, 2.5 mM and 10% DMSO) and 25 μL of human cathepsin B (4.0 nM) in assay buffer solution. The assay solutions were mixed for 5-10 seconds on a shaker plate and incubated for 15 minutes at room temperature. Z-Leu-Arg-AMC (8 μM) in 25 μL of assay buffer was added to the assay solutions. Hydrolysis of the coumarin leaving group (AMC) was followed by spectrofluorometry (Exλ=355 nm; Emλ=460 nm) for 10 minutes. Percent of inhibition were calculated by fitting experimental values to standard mathematical model for dose response curve.

### Cathepsin S Assay

Serial dilutions (1/3) from 500 μM down to 0.0085 uM of test compounds were prepared in dimethyl sulfoxide (DMSO). Then 2 μL of DMSO from each dilution were added to 50 μL of assay buffer (MES, 50 mM (pH 5.5); EDTA, 2.5 mM; DTT, 2.5 mM and 10% DMSO) and 25 μL of human cathepsin S (20 nM) in assay buffer solution. The assay solutions were mixed for 5-10 seconds on a shaker plate and incubated for 15 minutes at room temperature. Z-Leu-Arg-AMC (8 μM) in 25 μL of assay buffer was added to the assay solutions. Hydrolysis of the coumarin leaving group (AMC) was followed by spectrofluorometry (Exλ=355 nm; Emλ=−460 nm) for 10 minutes. Percent of inhibition were calculated by fitting experimental values to standard mathematical model for dose response curve.

### Pharmacokinetics in Rats

### Per Os (PO) Pharmacokinetics in Rats

### Procedure:

The animals are housed, fed and cared for according to the Guidelines of the Canadian Council on Animal Care.

Male Sprague Dawley rats (250-400 g) are fasted overnight prior to each PO blood level study.

The rats are placed in the restrainer one at a time and the box firmly secured. The zero blood sample is obtained by nicking a small (1 mm or less) piece off the tip of the tail. The tail is then stroked with a firm but gentle motion from the top to the bottom to milk out the blood. Approximately 0.5 mL of blood is collected into a heparinized vacutainer tube.

Compounds are prepared as required, in a standard dosing volume of 10 mL/kg, and administered orally by passing a 16 gauge, 3″ gavaging needle into the esophagus.

Subsequent blood collections are taken in the same manner as the zero blood sample except that there is no need to nick the tail again. The tail is cleaned with a piece of gauze and milked/stroked as described above into the appropriately labeled tubes.

Immediately after sampling, blood is centrifuged, separated, the plasma put into clearly marked vials and stored in a freezer until analyzed.

Typical time points for determination of rat blood levels after PO dosing are:
0, 15 min, 30 min, 1 h, 2 h, 4 h, 6 h, 8 h, 24 h

After the 4 hr time point bleed, food is provided to the rats ad libitum. Water is provided at all times during the study.

### Vehicles:

The following vehicles (with corresponding dose volumes) may be used in PO rat blood level determinations:

PEG 200/300/400 (0-60% in water): equal or less than 10 mL/kg
Methocel (0.5%-1.0% in water): equal or less than 10 mL/kg
Tween 80 (1-10% in water): equal or less than 10 mL/kg

Compounds for PO blood levels can be in suspension form. For better homogeneity, the suspension can be placed in a sonicator for approximately 5 minutes.

For analysis, aliquots are diluted with 1.2 to 1.5 volumes of acetonitrile optionally containing an internal standard and centrifuged to remove protein precipitate. The supernatant is injected directly onto a C-18 HPLC column with mass spectrometry (MS) or ultra-violet absorbance (UV) or fluorescence (Fluo) detection. Quantization is done relative to a standard curve prepared using clean blood samples spiked with a known quantities of drug in acetonitrile optionally containing an internal standard. Additional acetonitrile optionally containing internal standard is added to amount 1.2 to 1.5 volumes of the initial blood amount to correspond to what was done in the case of the samples. Bioavailability (F) is assessed by comparing area under the curve (AUC) i.v. versus p.o.

$$F = \frac{A U C po}{A U C iv} \times \frac{DOSEiv}{DOSEpo} \times 100 \%$$

$$and$$

$$A U C = ( C 1 + C 2 ) * ( T 2 - T 1 ) / 2$$

where C is the measured concentration by MS or UV or Fluo at a given time T

### Intravenous Pharmacokinetics in Rats

### Procedure:

The animals are housed, fed and cared for according to the Guidelines of the Canadian Council on Animal Care.

Male Sprague Dawley (325-375 g) non-fasted rats are used in theses studies.

The compound is prepared as required, in a standard dosing volume of 1 mL/kg.

Dosing of the conscious rats for intravenous administration is done via the jugular vein using a 25 gauge needle. This constitutes the zero time point.

The 5 min bleed is taken by nicking a piece (1-2 mm) off the tip of the tail. The tail is then stroked with a firm but gentle motion from the top of the tail to the bottom to milk the blood out of the tail. Approximately 0.5 mL of blood is collected into a heparinized collection vial. Subsequent bleeds are taken in the same fashion, except that there is no need to nick the tail again. The tail is cleaned with a piece of gauze and bled, as described above, into the appropriate labeled tubes.

Typical time points for determination of rat blood levels after I.V. dosing are either:
0, 5 min, 15 min, 30 min, 1 h, 2 h, 4 h, 6 h
or 0, 5 min, 30 min, 1 h, 2 h, 4 h, 6 h, 8 h, 24 h

### Vehicles:

The following vehicles may be used in IV rat blood level determinations:
Dextrose: 1 mL/kg
Moleculosol 25%: 1 mL/kg
DMSO (dimethylsulfoxide): Restricted 10% of the dose volume up to 0.1 mL per kilogram of animal
PEG 200: Not more than 80% mixed with 20% sterile water—1 mL/kg

With Dextrose, either sodium bicarbonate can be added if the solution is cloudy.

For analysis, aliquots are diluted with 1.2 to 1.5 volumes of acetonitrile optionally containing an internal standard and centrifuged to remove protein precipitate. The supernatant is injected directly onto a C-18 HPLC column with mass spectrometry (MS) or ultra-violet absorbance (UV) or fluorescence (Fluo) detection. Quantization is done relative to a standard curve prepared using clean blood samples spiked with a known quantities of drug in acetonitrile optionally containing an internal standard. Additional acetonitrile optionally containing internal standard is added to amount 1.2 to 1.5 volumes of the initial blood amount to correspond to what was done in the case of the samples. Bioavailability (F) is assessed by comparing area under the curve (AUC) i.v. versus p.o.

$$F = \frac{A U C po}{A U C iv} \times \frac{DOSEiv}{DOSEpo} \times 100 \%$$

$$and$$

$$A U C = ( C 1 + C 2 ) * ( T 2 - T 1 ) / 2$$

where C is the measured concentration by MS or UV or Fluo at a given time T.

### Hepatocyte Incubations

For rat hepatocyte incubations, 1×10⁶ cells diluted in 0.5 mL of Krebs-Henseleit buffer were first prepared at 37° C. for 20 min under 95%:5% O₂:CO₂ (BOC gases: Montreal, Canada) in a 48-well plate, and the 5 mL of a 10 mM solution of compound dissolved in acetonitrile were added to each well to a final concentration of 50 μM. After 2 h of incubation at 37° C. under 95%:5% O₂:CO₂ atmosphere, one volume of acetonitrile was added in each well. A quenched incubation spiked with the parent compound and a blank were also prepared as controls. Once transferred, samples were centrifuged for 10 min at 14,000 rpm using an Eppendorf 5415C centrifuge (Hamburg, Germany) and the supernatant used for LC/UV/MS analysis.

1. A compound of the formula: 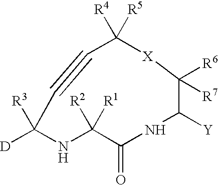
wherein Y is hydrogen, CN, —C(O)R⁸, —C(O)NR⁸R⁹, —CH₂OH, —C(O)NR⁸OR⁹, or —C(O)OR⁸;
X is S(O)ₘ, —CH₂—, —OC(O)— or —C(O)O—;
R¹ is hydrogen, C₁₋₆ alkyl or C₂₋₆ alkenyl wherein said alkyl and alkenyl groups are optionally substituted with SO₂R¹⁰, C₃₋₆ cycloalkyl or halo;
R² is hydrogen, C₁₋₆ alkyl or C₂₋₆ alkenyl wherein said alkyl and alkenyl groups are optionally substituted with SO₂R¹⁰, C₃₋₆ cycloalkyl or halo;
or R¹ and R² can be taken together with the carbon atom to which they are attached to form a C₃₋₈ cycloalkyl ring, C₅₋₈ cycloalkenyl ring, or five to seven membered heterocyclyl wherein said cycloalkyl, cycloalkenyl and heterocyclyl groups are optionally substituted on either the carbon or heteroatom with C₁₋₆ alkyl, halo, hydroxyalkyl, hydroxy, alkoxy or keto;
R³ is C₁₋₆ alkyl substituted with 1-6 halo;
R⁴ is hydrogen, C₁₋₆ alkyl or C₂₋₆ alkenyl wherein said alkyl and alkenyl groups are optionally substituted with C₃₋₆ cycloalkyl or halo;
R⁵ is hydrogen, C₁₋₆ alkyl or C₂₋₆ alkenyl wherein said alkyl and alkenyl groups are optionally substituted with C₃₋₆ cycloalkyl or halo;
or R⁴ and R⁵ can be taken together with the carbon atom to which they are attached to form a C₃₋₈ cycloalkyl ring, C₅₋₈ cycloalkenyl ring, or five to seven membered heterocyclyl wherein said cycloalkyl, cycloalkenyl and heterocyclyl groups are optionally substituted on either the carbon or heteroatom with C₁₋₆ alkyl, halo, hydroxyalkyl, hydroxy, alkoxy or keto;
R⁶ is hydrogen, C₁₋₆ alkyl or C₂₋₆ alkenyl wherein said alkyl and alkenyl groups are optionally substituted with C₃₋₆ cycloalkyl or halo;
R⁷ is hydrogen, C₁₋₆ alkyl or C₂₋₆ alkenyl wherein said alkyl and alkenyl groups are optionally substituted with C₃₋₆ cycloalkyl or halo;
or R⁶ and R⁷ can be taken together with the carbon atom to which they are attached to form a C₃₋₈ cycloalkyl ring, C₅₋₈ cycloalkenyl ring, or five to seven membered heterocyclyl wherein said cycloalkyl, cycloalkenyl and heterocyclyl groups are optionally substituted on either the carbon or heteroatom with C₁₋₆ alkyl, halo, hydroxyalkyl, hydroxy, alkoxy or keto;
R⁸ is hydrogen, C₁₋₆ alkyl or C₂₋₆ alkenyl wherein said alkyl and alkenyl groups are optionally substituted with C₃₋₆ cycloalkyl or halo;
R⁹ is hydrogen, C₁₋₆ alkyl or C₂₋₆ alkenyl wherein said alkyl and alkenyl groups are optionally substituted with C₃₋₆ cycloalkyl or halo;
or R⁸ and R⁹ can be taken together with the atoms to which they are attached or are between them to form a C₃₋₈ cycloalkyl ring, C₅₋₈ cycloalkenyl ring, or five to seven membered heterocyclyl wherein said cycloalkyl, cycloalkenyl and heterocyclyl groups are optionally substituted on either the carbon or heteroatom with C₁₋₆ alkyl, halo, hydroxyalkyl, hydroxy, alkoxy or keto;
R¹⁰ is C₁₋₆ alkyl, C₁₋₆ haloalkyl, C₃₋₆ cycloalkyl, C₃₋₆ cycloalkyl(C₁₋₆ alkyl), aryl, aryl(C₁₋₆ alkyl), heteroaryl or heteroaryl(C₁₋₆ alkyl), wherein said cycloalkyl group is optionally substituted with C₁₋₆ haloalkyl, and wherein said aryl and heteroaryl groups are optionally substituted with 1 to 3 substituents independently selected from C₁₋₆ alkyl, halo, C₁₋₆ haloalkyl, —SR^{a}, —S(O)R^{a}, —S(O)₂R^{a}, —OR^{a}, NR^{c}R^{d}, cyano or aryl;
R^{a} is hydrogen, C₁₋₆ alkyl, aryl, heteroaryl, aryl(C₁₋₆ alkyl), or heteroaryl(C₁₋₆ alkyl);
R^{b} is hydrogen or C₁₋₆ alkyl;
R^{c} is hydrogen or C₁₋₆ alkyl;
R^{d} is hydrogen or C₁₋₆ alkyl;
or R^{c} and R^{d} can be taken together with the nitrogen atom to which they are attached to form a four to six membered heterocyclyl which may contain a second heteroatom selected from O, S, NH or NC₁₋₆ alkyl;
D is independently hydrogen, C₂₋₆ alkynyl, aryl, heteroaryl, C₃₋₈ cycloalkyl or heterocyclyl wherein said alkynyl, aryl, heteroaryl, cycloalkyl and heterocyclyl groups, which may be monocyclic or bicyclic, are optionally substituted on either the carbon or the heteroatom with one to five R¹¹;
R¹¹ is hydrogen, C₁₋₆ alkyl, C₂₋₆ alkenyl, C₂₋₆ alkynyl, C₁₋₆ alkyloxy, halo, nitro, cyano, aryl, heteroaryl, C₃₋₈ cycloalkyl, heterocyclyl, —C(O)OR¹³, —OR¹⁵, —OR¹³, —C(O)R¹³, —R¹³C(O)R¹⁵, —C(O)N(R^{a})(R^{b}), —C(O)N(R¹³)(R¹⁴), —C(R¹³)(R¹⁴)OH, —R¹⁵, —C(R¹³)(R¹⁴)N(R¹⁵)₂, —NR¹⁰C(O)NR¹³S(O)₂R¹⁵, —SO₂R¹², —SO(R¹²), —SO₂R¹⁵, —SOₘN(R^{c})(R^{d}), —SOₘCH(R¹³)(R¹⁴), —SO₂N(R¹³)C(O)(R¹²), —N(R¹³)(R¹⁴), —N(R¹³)C(O)N(R¹³)(R¹⁵), —N(R¹³)C(O)R¹⁵, —N(R¹³)C(O)R¹³, —N(R¹³)C(O)OR¹³, —N(R¹³)SO₂(R¹³), —C(O)C(R^{a})(R^{b})N(R^{e})(R^{d}), —C(R^{a})(R^{b})N(R^{c})C(O)R¹⁵, —C(O)C(R^{a})(R^{b})S(R^{a}), C(R^{a})(R^{b})C(O)N(R^{c})(R^{d}); wherein said groups are optionally substituted on either the carbon or the heteroatom with one to five substituents independently selected from C₁₋₆ alkyl, halo, keto, cyano, C₁₋₆ haloalkyl, hydroxyalkyl, —OR¹⁵, —NO₂, —NH₂, —NHS(O)₂R¹³, —R¹⁵SO₂R¹², —SO₂R¹², —SO(R¹²), —SR¹², —SR¹⁵, —SOₘN(R^{c})(R^{d}), —SOₘN(R¹³)C(O)(R¹²), —C(R¹³)(R¹⁴)N(R¹³)(R¹⁴), —C(R¹³)(R¹⁴)OH, —COOH, —C(R^{a})(R^{b})C(O)N(R^{c})(R^{d}), —C(O)(R^{a})(R^{b}), —N(R¹³)C(R¹³)(R¹⁴)(R¹⁵), —N(R¹³)CO(R¹⁵), —NH(CH₂)₂OH, —NHC(O)OR¹³, heterocycyl, aryl, or heteroaryl;
R¹² is hydrogen or C₁₋₆ alkyl which is optionally substituted with one, two, or three substituents independently selected from halo, alkoxy, cyano, —NR¹³ or —SR¹³;
R¹³ is hydrogen or C₁₋₆ alkyl;
R¹⁴ is hydrogen or C₁₋₆ alkyl;
R¹⁵ is hydrogen, aryl, aryl(C₁₋₄) alkyl, heteroaryl, heteroaryl(C₁₋₄)alkyl, C₃₋₈cycloalkyl, C₃₋₈ cycloalkyl(C₁₋₄)alkyl or heterocyclyl(C₁₋₄)alkyl wherein said groups can be optionally substituted with one, two, or three substituents independently selected from halo, alkoxy or —SO₂R¹²;
m is 0, 1, or 2;
or a pharmaceutically acceptable salt, stereoisomer or N-oxide derivative thereof.

2. The compound of claim 1 wherein R⁴ is hydrogen or C₁₋₃ alkyl; R⁵ is hydrogen; or a pharmaceutically acceptable salt, stereoisomer or N-oxide derivative thereof.

3. The compound of claim 2 wherein R⁶ is hydrogen; R⁷ is hydrogen; or a pharmaceutically acceptable salt, stereoisomer or N-oxide derivative thereof.

4. The compound of claim 3 wherein R³ is C₁₋₃ alkyl substituted with three halo; or a pharmaceutically acceptable salt, stereoisomer or N-oxide derivative thereof.

5. The compound of claim 4 wherein R³ is trifluoromethyl; or a pharmaceutically acceptable salt, stereoisomer or N-oxide derivative thereof.

6. The compound of claim 5 wherein X is S; or a pharmaceutically acceptable salt, stereoisomer or N-oxide derivative thereof.

7. The compound of claim 1 which is:
(3R,6S,8R)-8-(4-bromophenyl)-6-(2-fluoro-2-methylpropyl)-5-oxo-8-(trifluoromethyl)-1-thia-4,7-diazacycloundec-9-yne-3-carbonitrile 1,1-dioxide;
(3R,6S,8R)-8-(1-benzothien-2-yl)-6-isobutyl-5-oxo-8-(trifluoromethyl)-1-thia-4,7-diazacycloundec-9-yne-3-carbonitrile;
(3R,6S,8R)-3-acetyl-8-(4-bromophenyl)-6-(2-fluoro-2-methylpropyl)-8-(trifluoromethyl)-1-thia-4,7-diazacycloundec-9-yn-5-one 1,1-dioxide;
(3R,6S,8R)-3-acetyl-8-(4-bromophenyl)-6-(2-fluoro-2-methylpropyl)-8-(trifluoromethyl)-1-thia-4,7-diazacycloundec-9-yn-5-one;
(3R,6S,8R)-8-(1-benzothien-2-yl)-6-isobutyl-5-oxo-8-(trifluoromethyl)-1-thia-4,7-diazacycloundec-9-yne-3-carboxamide;
(3R,6S,8R)-8-(4-bromophenyl)-11-ethyl-6-(2-fluoro-2-methylpropyl)-5-oxo-8-(trifluoromethyl)-1-thia-4,7-diazacycloundec-9-yne-3-carbonitrile;
(2S,5S,11R)-11-(4-bromophenyl)-2-(2-fluoro-2-methylpropyl)-3-oxo-11-(trifluoromethyl)-1,4-diazacycloundec-9-yne-5-carbonitrile;
(3R,6S,8R)-8-(4-bromophenyl)-11-ethyl-6-(2-fluoro-2-methylpropyl)-5-oxo-8-(trifluoromethyl)-1-thia-4,7-diazacycloundec-9-yne-3-carboxamide;
(2S,5S,11R)-11-(4-bromophenyl)-2-(2-fluoro-2-methylpropyl)-3-oxo-11-(trifluoromethyl)-1,4-diazacycloundec-9-yne-5-carboxamide;
(3S,5R,11S)-5-(4-bromophenyl)-3-(2-fluoro-2-methylpropyl)-11-(hydroxymethyl)-5-(trifluoromethyl)-1,4-diazacycloundec-6-yn-2-one;
(2S,5S,11R)-11-(4-bromophenyl)-2-(2-fluoro-2-methylpropyl)-3-oxo-11-(trifluoromethyl)-1,4-diazacycloundec-9-yne-5-carboxylic acid;
(4S,7S,9R)-9-(4-bromophenyl)-7-isobutyl-2,6-dioxo-9-(trifluoromethyl)-1-oxa-5,8-diazacyclododec-10-yne-4-carbonitrile;
(3R,6S,8R)-8-(6-bromo-2-naphthyl)-6-isobutyl-5-oxo-8-(trifluoromethyl)-1-thia-4,7-diazacycloundec-9-yne-3-carbonitrile;
(3R,6S,8R)-8-(6-bromo-2-naphthyl)-6-isobutyl-5-oxo-8-(trifluoromethyl)-1-thia-4,7-diazacycloundec-9-yne-3-carboxamide;
(4S,7S,9R)-9-(4-bromophenyl)-7-isobutyl-2,6-dioxo-9-(trifluoromethyl)-1-oxa-5,8-diazacyclododec-10-yne-4-carboxamide;
(3R,6S,8R)-6-(2-fluoro-2-methylpropyl)-8-[4′-(methylsulfonyl)biphenyl-4-yl]-5-oxo-8-(trifluoromethyl)-1-thia-4,7-diazacycloundec-9-yne-3-carbonitrile;
1-{4′-[(3R,6S,8R)-3-cyano-6-(2-fluoro-2-methylpropyl)-5-oxo-8-(trifluoromethyl)-1-thia-4,7-diazacycloundec-9-yn-8-yl]biphenyl-4-yl}cyclopropanecarboxamide;
(3R,6S,8R)-8-biphenyl-4-yl-6-(2-fluoro-2-methylpropyl)-5-oxo-8-(trifluoromethyl)-1-thia-4,7-diazacycloundec-9-yne-3-carbonitrile;
(3R,6S,8R)-8-(4-bromophenyl)-6-(2-fluoro-2-methylpropyl)-5-oxo-8-(trifluoromethyl)-1-thia-4,7-diazacycloundec-9-yne-3-carbonitrile 1-oxide;
(3R,6S,8R)-8-(4-bromophenyl)-6-(2-fluoro-2-methylpropyl)-5-oxo-8-(trifluoromethyl)-1-thia-4,7-diazacycloundec-9-yne-3-carbonitrile;
(3R,6S,8R)-6-(2-fluoro-2-methylpropyl)-5-oxo-8-(4′-piperazin-4-ium-1-ylbiphenyl-4-yl)-8-(trifluoromethyl)-1-thia-4,7-diazacycloundec-9-yne-3-carboxylate;
(3R,6S,8R)-3-acetyl-8-(4-bromophenyl)-6-(2-methylprop-2-en-1-yl)-8-(trifluoromethyl)-1-thia-4,7-diazacycloundec-9-yn-5-one;
(3R,6S,8R)-8-(4-bromophenyl)-6-(2-fluoro-2-methylpropyl)-N-methoxy-N-methyl-5-oxo-8-(trifluoromethyl)-1-thia-4,7-diazacycloundec-9-yne-3-carboxamide;
(3R,6S,8R)-8-(4-bromophenyl)-6-(2-fluoro-2-methylpropyl)-5-oxo-8-(trifluoromethyl)-1-thia-4,7-diazacycloundec-9-yne-3-carboxylic acid;
(3R,6S,8R)-8-(4-bromophenyl)-6-(2-fluoro-2-methylpropyl)-3-(hydroxymethyl)-8-(trifluoromethyl)-1-thia-4,7-diazacycloundec-9-yn-5-one;
methyl (3R,6S,8R)-8-(4-bromophenyl)-6-(2-fluoro-2-methylpropyl)-5-oxo-8-(trifluoromethyl)-1-thia-4,7-diazacycloundec-9-yne-3-carboxylate;
(3R,6S,8R)-6-(2-fluoro-2-methylpropyl)-8-[4′-(methylsulfonyl)biphenyl-4-yl]-5-oxo-8-(trifluoromethyl)-1-thia-4,7-diazacycloundec-9-yne-3-carboxamide;
(3R,6S,8R)-8-(4-bromophenyl)-6-(2-fluoro-2-methylpropyl)-5-oxo-8-(trifluoromethyl)-1-thia-4,7-diazacycloundec-9-yne-3-carboxamide;
(3R,6S,8R)-6-isobutyl-8-[4′-(methylsulfonyl)biphenyl-4-yl]-5-oxo-8-(trifluoromethyl)-1-thia-4,7-diazacycloundec-9-yne-3-carbonitrile;
1-{4′-[(3R,6S,8R)-3-cyano-6-isobutyl-5-oxo-8-(trifluoromethyl)-1-thia-4,7-diazacycloundec-9-yn-8-yl]biphenyl-4-yl}cyclopropanecarboxamide;
(3R,6S,8R)-8-(4-bromophenyl)-6-isobutyl-5-oxo-8-(trifluoromethyl)-1-thia-4,7-diazacycloundec-9-yne-3-carbonitrile;
(3R,6S,8R)-8-(4-bromophenyl)-6-isobutyl-5-oxo-8-(trifluoromethyl)-1-thia-4,7-diazacycloundec-9-yne-3-carboxamide;
or a pharmaceutically acceptable salt, stereoisomer or N-oxide derivative thereof.

8. A pharmaceutical composition comprising a compound according to claim 1, or a pharmaceutically acceptable salt, stereoisomer or N-oxide derivative thereof, and a pharmaceutically acceptable carrier.

9. A method of treating osteoporosis, glucocorticoid induced osteoporosis, Paget's disease, abnormally increased bone turnover, periodontal disease, tooth loss, bone fractures, rheumatoid arthritis, osteoarthritis, periprosthetic osteolysis, osteogenesis imperfecta, atherosclerosis, obesity, glaucoma, chronic obstructive pulmonary disease, metastatic bone disease, hypercalcemia of malignancy, multiple myeloma, Alzheimer's disease, neuropathic and inflammatory pain, diabetes, juvenile onset diabetes, multiple sclerosis, pemphigus vulgaris, Graves' disease, myasthenia gravis, systemic lupus erythemotasus, Hashimoto's thyroiditis, asthma; rejection of organ transplants, rejection of tissue grafts, tumor invasion, metastasis, pneumocystis carinii, acute pancreatitis, liver disease, stroke, inflammatory airway disease or inflammatory bowel disease in a mammal in need thereof with a therapeutically effective amount of a compound according to claim 1.

10. A pharmaceutical composition comprising a compound of claim 1, or a pharmaceutically acceptable salt, stereoisomer or N-oxide derivative thereof, and another agent selected from the group consisting of: an organic bisphosphonate, a selective estrogen receptor modulator, an estrogen receptor beta modulator, an androgen receptor modulator, an inhibitor of osteoclast proton ATPase, an inhibitor of HMG-CoA reductase, an integrin receptor antagonist, or an osteoblast anabolic agent, vitamin D, a synthetic Vitamin D analogue, a Nonsteroidal anti-inflammatory drug, a selective cyclooxygenase-2 inhibitor, an inhibitor of interleukin-1 beta, a LOX/COX inhibitor and the pharmaceutically acceptable salts and mixtures thereof.

11. The method of claim 9 which further comprises a therapeutically effective amount of another agent selected from the group consisting of: an organic bisphosphonate, a selective estrogen receptor modulator, an androgen receptor modulator, an inhibitor of osteoclast proton ATPase, an inhibitor of HMG-CoA reductase, an integrin receptor antagonist, an osteoblast anabolic agent, vitamin D, a synthetic Vitamin D analogue, a Nonsteroidal anti-inflammatory drug, a selective cyclooxygenase-2 inhibitor, an inhibitor of interleukin-1 beta, a LOX/COX inhibitor and the pharmaceutically acceptable salts and mixtures thereof.
